(19) United States
(12) Patent Application Publication
(10) Pub. No.: US 2009/0203541 A1
(43) Pub. Date: Aug. 13, 2009

(54) MSP AND ITS DOMAINS AS FRAMEWORKS FOR NOVEL BINDING MOLECULES

(76) Inventor: JAMES W. WEST, SANTA BARBARA, CA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(21) Appl. No.: 12/369,900

(22) Filed: Feb. 12, 2009

Related U.S. Application Data
(60) Provisional application No. 61/027,995, filed on Feb. 12, 2008.

(51) Int. Cl. C40B 30/04; C12N 15/12; C12N 15/85; C12N 5/10; C40B 40/10; C12Q 1/68; C07K 14/47

(52) U.S. Cl. 506/9; 530/350; 536/23.5; 435/320.1; 435/325; 506/18; 435/6; 530/324

(57) ABSTRACT

Disclosed are compositions and methods relating to the use of MSP94 proteins and its domains as frameworks for novel binding molecules, including screening assays for the identification of novel binding molecules.

### CROSS-REFERENCES TO RELATED APPLICATIONS

This application claims the benefit of U.S. Provisional Patent Application No. 61/027,995, filed Feb. 12, 2008, which is incorporated by reference herein in its entirety.

### BACKGROUND OF THE INVENTION

### β-microseminoprotein (MSP)

β-microseminoprotein (MSP), also called the prostate secretory protein of 94 amino acids (PSP₉₄), is a small, unglycosylated protein first identified in seminal serum. (See Thakur et al., Indian J. Exp. Biol. 19:307-313, 1981.) A variety of roles and potential uses of this protein have been proposed, resulting in its designation by other names such as immunoglobulin-binding factor (see Liang et al., Biochem. Biophys. Res. Comm. 180:356-359, 1991) and prostatic inhibin peptide (see Garde et al., Prostate 22:225-233, 1993). Although primarily expressed in the prostate and found in prostatic secretions of various mammals, MSP has been more recently identified in other tissues, including nonreproductive organs such as the gastrointestinal tract (see, e.g., Weiber et al., Digestion 60:440-448, 1999; Ulvsbäck et al., Biochem. Biophys. Res. Comm. 164:1310-1315, 1989) and the respiratory tract (see, e.g., Ulvsbäck et al., supra; Weiber et al., Am. J. Pathol. 137:593-603, 1990), as well as various female tissues (see, e.g., Tanaka et al., Mol. Reprod. Dev. 42:149-156, 1995 (porcine ovarian lutein cells); Baijal-Gupta et al, J. Endocrinol. 165:425-433, 2000 (female reproductive tissues, breast, and endometrial cancer cell lines)).

Although studies have failed to unambiguously identify a biological role for MSP, studies suggest that this protein has rapidly evolved. (See Nolet et al., Genomics 9:775-777, 1991; Fernlund et al., Arch. Biochem. Biophys. 334:73-82, 1996; Mäkinen et al., Eur. J. Biochem. 264:407-414, 1999.) The primary structure of MSP shows a remarkably low level of conservation in amino acids among the species studied, often resulting in great variation of physico-chemical properties. Indeed, the great number of amino acid substitutions in these proteins has for years rendered the identification of MSPs using immunological or hybridization techniques difficult in more distant species, and a comparison of known sequences from human (Mbikay et al., DNA 6:23-29, 1987), rhesus monkey (Nolet et al, supra), baboon (Xuan et al, DNA Cell Biol. 16:627-638, 1997), cotton-top tamarin (Mäkinen et al., supra), pig (Tanaka et al., supra), rat (Fernlund et al., supra), and mice (Xuan et al., DNA Cell Biol 18:11-26, 1999) reveals that, apart from the ten completely conserved half-cysteine residues, these protein share very few other conserved residues. (Lazure et al., Protein Sci. 10:2207-2218, 2001.)

### Protein Display Scaffolds

Antibodies, as a source of diversified proteins employed by nature for an adaptive immune response, have been the natural prototype of specifically binding proteins used as diversity-carrying scaffolds for library construction during the last decades. In many applications, however, the constant regions of whole antibodies are not necessarily required. Thus, there has been an increasing emphasis on systems based on antibody fragments (see Holliger and Hudson, Nat. Biotechnol 23:1126-1136, 2005), a trend which is also reflected by the variety of small scaffolds of nonimmunoglobulin origin ranging from 263 residues for TEM-1 β-lactamase (see Legendre et al., Protein Sci. 11:1506-1518, 2002) to 23 residues for Min-23 (see Souriau et al., Biochemistry 44:7143-7155, 2005). At present, domain antibodies (dabs) are the smallest known antigen-binding fragments of antibodies (see Holt et al., Trends Biotechnol 21:484-490, 2003). Furthermore, antibody engineering is expanding to novel antibody lineages of sharks (see Nuttall et al., Mol Immunol 38:313-326, 2001; Dooley et al., Mol Immunol 40:25-33, 2003) and camels (see Omidfar et al., Tumour Biol 25:296-305, 2004; Rahbarizadeh et al., Hybrid. Hybridomics 22:151-159, 2004).

While the biotechnological and clinical applications of the diverse antibody formats are numerous, there is increasing emphasis on nonimmunoglobulin protein scaffolds. The emerging field of protein engineering has led to a wide range of different nonimmunoglobulin scaffolds with widely diverse origins and characteristics. More than 30 of such scaffolds have been used as alternatives to antibodies for the construction of protein libraries and grafting experiments (see Binz and Plückthun, Curr. Opin. Biotechnol 16:459-469, 2005). Some of them are comparable in size to a scFv of an antibody (˜30 kDa) (e.g., TEM-1 β-lactamase, T-cell receptors, or green fluorescent protein), while the majority of them are much smaller. The smallest scaffolds include the knottin Min-23 (23 residues), a designed version of insect defensin A (29 residues), or the scorpion toxin charybdotoxin (37 residues). In contrast, modular scaffolds based on repeat proteins, e.g., the 33-residue ankyrin repeat motif, vary in size depending on the number of repetitive units.

There is a need in the art for additional protein scaffolds for library construction and generation of novel binding molecules. Such increased variety of alternative scaffold structures will provide a solid foundation for the successful isolation and exploitation of high-affinity and high avidity binders in the fields of research, diagnosis, and therapy.

### BRIEF SUMMARY OF THE INVENTION

In one aspect, the present invention provides a polypeptide comprising an MSP monomer domain I variant and/or an MSP monomer domain II variant. Typically, the MSP monomer domain I variant is characterized in that (a) in its unmodified form, the MSP monomer domain I comprises the amino acid sequence (x₁)C₂{x₃₋₇}{Z_{I}}C₁₈{x₁₉₋₃₆}C₃₇x₃₈x₃₉C₄₀x₄₁C₄₂{Z_{II}}x₄₈C₄₉C₅₀ (x₅₁₋₅₅) (SEQ ID NO:59), which represents a polypeptide region from a wild-type MSP protein corresponding to amino acid residues 1 to 55 of human MSP (SEQ ID NO:4), where {Z_{II}} and {Z_{II}} denote loop regions corresponding, respectively, to amino acids 8-17 and 43-47 of human MSP; and (b) in its modified form, the MSP monomer domain I variant (i) includes cysteine residues C₂, C₁₈, C₄₀, C₄₂, C₄₉, and C₅₀; (ii) includes either or both of a modified {Z_{II}} and a modified {Z_{II}} loop region, each modified loop region being a heterologous polypeptide segment of from 4 to 30 amino acids; and (iii) includes an amino acid sequence at least 60% identical to the wild-type MSP polypeptide region, exclusive of {Z_{I}} and {Z_{II}}. Typically, the MSP monomer domain II variant is characterized in that (a) in its unmodified form, the MSP monomer domain II comprises the amino acid sequence {x₅₄₋₅₆}{Z_{III}}C₆₄{x₆₅₋₇₂}C₇₃{x₇₄₋₇₇}{Z_{IV}}x₈₆C₈₇({x₈₈₋₉₄}) (SEQ ID NO:60), which represents a polypeptide region from a wild-type MSP protein corresponding to amino acid residues 52 to 94 of human MSP (SEQ ID NO:4), where {Z_{III}} and {Z_{IV}} denote loop regions corresponding, respectively, to amino acids 57-63 and 78-85 of human MSP; and (b) in its modified form, the MSP monomer domain II variant (i) includes cysteine residues C₆₄ and C₈₇; (ii) includes either or both of a modified {Z_{III}} and a modified {Z_{IV}} loop region, where each modified loop region is a heterologous polypeptide segment of from 4 to 30 amino acids; and (iii) includes an amino acid sequence at least 60% identical to the wild-type MSP polypeptide region, exclusive of {Z_{II}} and {Z_{IV}}. In exemplary embodiments, the wild-type MSP protein is the human MSP protein of SEQ ID NO:4.

In certain embodiments, a polypeptide as above comprises one MSP monomer domain variant. Alternatively, the polypeptide can comprise a plurality of MSP monomer domain variants, each MSP monomer domain variant independently selected from the domain I variant and the domain II variant. In some variations, where the polypeptide comprises the MSP domain I variant, a non-sulfhydryl-containing amino acid residue is substituted for C₃₇; and where the polypeptide comprises the MSP domain II variant, a non-sulfhydryl-containing amino acid residue is substituted for C₇₃.

In some embodiments, a polypeptide comprising an MSP domain variant includes both an MSP domain I variant and an MSP domain II variant. In certain variations, the domain II variant is linked C-terminal and directly adjacent to the domain I variant. The domain I and domain II variants can be linked, for example, via a polypeptide linker heterologous to the wild-type MSP protein. Alternatively, the domain I and domain II variants may be linked via the natural connection between domain I and domain II. In some embodiments, the domain I variant includes cysteine C₃₇ and the domain II variant includes cysteine C₇₃. In specific variations, a polypeptide comprising both domain I and domain II comprises the amino acid sequence (S)CYFIPN{Z_{I}}CMDLKGNKHPINSEW QTDNxETCTC{Z_{II}}SCCTLVSTP{Z_{III}}CQRIFKKEDxKYIV{Z_{IV}} TC(SVSEWII) (SEQ ID NO:72) or the amino acid sequence (S)CYFIPN{Z_{I}}CMDLKGNKHPINSEWQTDNCETCTC {Z_{II}}SCCTLVS TP{Z_{III}}CQRIFKKEDCKYIV {Z_{IV}}TC(SVSEWII) (SEQ ID NO:73).

In some embodiments, a polypeptide comprising an MSP domain variant includes the domain I variant. Such polypeptides can, for example, include both the modified {Z_{I}} and modified {Z_{II}} loop regions or only one of the modified {Z_{I}} and the modified {Z_{II}} loop regions. In specific variations, the domain I variant comprises an amino acid sequence selected from the following:

(SEQ ID NO:74)

(x)Cxxxxx{Z_{I}}CxDxxGxxHxxxxxWx(x)xxxxxCxC{Z_{II}}xCC
(xxxxx);

(SEQ ID NO:75)

(x)C[yfs][fliqt][imqek][prnil][nlrp]{Z_{I}}CxDx[kd]
GxxHxx[ndg][ste]xWx([tn])xxxxxCxC{Z_{II}}[sitra]CC
(xxxxx);

(SEQ ID NO:76)

(x)C[ys][fli][imqe][prn][nlr]{Z_{I}}CxDx[kd]GxxHx
[il][nd][st]xW[qkr](T)[dek]xxxxCxC{Z_{II}}[sit]C
C(xxxxx);

(SEQ ID NO:77)

(S)CxxxPN{Z_{I}}CxDLKGNKHPxxSxWxTxxxxxCxC{Z_{II}}xCC
([ts]Lxx[ti]);

(SEQ ID NO:78)

(S)C[ys][fl][im]PN{Z_{I}}C[mt]DLKGNKHP[il][nd]S[ekr]
W[qkr]T[de][nd]x[de]xCxC{Z_{II}}[si]CC([ts]L[vi][sa]
[ti]);

(SEQ ID NO:79)

(S)CYFIPN{Z_{I}}CMDLKGNKHPINSEWQTDNxETCTC{Z_{II}}SCC
(TLVST).

In some embodiments, a polypeptide comprising an MSP domain variant includes the domain II variant. Such polypeptides can, for example, include both the modified {Z_{III}} and modified {Z_{IV}} loop regions or only one of the modified {Z_{III}} and the modified {Z_{IV}} loop regions. In specific variations, the domain I variant comprises an amino acid sequence selected from the following:

(SEQ ID NO:81)

xxx{Z_{III}}CxxxFxxxxxxxxV{Z_{IV}}xC(xxxxxxx);

(SEQ ID NO:82)

xxx{Z_{III}}C[qrd][rkv][iq]F[knh]x[ek]xx[krt][yi]
[ist]V{Z_{IV}}xC(x[vi]xxWxx);

(SEQ ID NO:83)

xxx{Z_{III}}CxxIFxxExxxxxV{Z_{IV}}xC(xxxxxxx);

(SEQ ID NO:84)

[sa][ti]P{Z_{III}}C[qr][rk]xF[kn][kq]x[det]x[kr][yi]
[is]V{Z_{IV}}[te]C(x[vi]xxxxx);

(SEQ ID NO:85)

xTP{Z_{III}}CQRIFKKExxKYIV{Z_{IV}}TC(xxxxWIx);

(SEQ ID NO:86)

[sa]TP{Z_{III}}CQRIFKKE[de]xKYIV{Z_{IV}}TC(xxx[eq]WI
[il]);

(SEQ ID NO:87)

STP{Z_{III}}C64QRIFKKEDx73KYIV{Z_{IV}}TC(SVSEWII).

In certain embodiments of a polypeptide comprising an MSP monomer domain I variant and/or an MSP monomer domain II variant as above, the domain I variant further includes a modified loop region {Z_{V}} at a region corresponding to amino acid residues 21-25 of human MSP. Accordingly, in some variations, an MSP monomer domain I variant is characterized in that (a) in its unmodified form, the MSP monomer domain I comprises the amino acid sequence (x₁)C₂ {x₃₋₇}{Z}C₁₈x₁₉x₂₀{Z_{V}}{x₂₆₋₃₆}C₃₇x₃₈x₃₉C₄₀x₄₁C₄₂{Z_{II}}x₄₈C₄₉C₅₀(x₅₁₋₅₅) (SEQ ID NO:59), which represents a polypeptide region from a wild-type MSP protein corresponding to amino acid residues 1 to 55 of human MSP (SEQ ID NO:4), where {Z_{I}}, {Z_{II}}, and {Z_{V}} denote loop regions corresponding, respectively, to amino acids 8-17, 43-47, and 21-25 of human MSP; and (b) in its modified form, the MSP monomer domain I variant (i) includes cysteine residues C₂, C₁₈, C₄₀, C₄₂, C₄₉, and C₅₀; (ii) includes at least one of a modified {Z_{I}}, modified {Z_{II}}, and modified {Z_{V}} loop region, each modified loop region being a heterologous polypeptide segment of from 4 to 30 amino acids; and (iii) includes an amino acid sequence at least 60% identical to the wild-type MSP polypeptide region, exclusive of {Z_{I}}, {Z_{II}}, and {Z_{V}}. In particular variations of a polypeptide comprising both a domain I variant and a domain II variant, the polypeptide includes the amino acid sequence (S)CYFIPN{Z_{I}}CMD {Z_{V}}HPINSEWQTDNxETCTC {Z_{II}} SCCTLVSTP {Z_{III}}CQRIFKKEDxKYIV{Z_{IV}}TC(SVSEWII) (SEQ ID NO:89) or the amino acid sequence (S)CYFIPN{Z_{I}}CMD{Z_{V}}H PINSEWQTDNCETCTC{Z_{II}}SCCTLVSTP{Z_{II}}CQRIFKKEDCKYIV {ZIV}TC(SVSEWII) (SEQ ID NO:90).

In another aspect, the present invention provides polynucleotides encoding a polypeptide as above, as well as vectors comprising such polynucleotides. In particular embodiments, a vector of the invention is an expression vector such as, for example, a phage display vector. Also provided are host cells comprising such vectors.

In yet another aspect, the present invention provides a polypeptide library comprising a pool of different polypeptides, where each polypeptide of the polypeptide pool is a polypeptide as described above; and where {Z_{I}}, {Z_{II}}, {Z_{III}}, {Z_{IV}}, and/or {Z_{V}} is different among different polypeptides of the polypeptide pool. In certain embodiments, each polypeptide of the polypeptide pool comprises one MSP monomer domain variant (i.e., either a domain I variant or a domain II variant). Alternatively, each polypeptide may include at least two MSP monomer domain variants. In some such embodiments, each of the at least two MSP monomer domain variants is a domain I variant or each of the at least two MSP monomer domain variants is a domain II variant. In other such embodiments comprising at least two MSP monomer domain variants, each polypeptide includes both a domain I variant and a domain II variant.

In still another aspect, the present invention provides a method of screening a MSP monomer domain variant for the ability to bind to a target molecule. The method generally includes the following steps: (1) contacting a first target molecule with a MSP monomer domain variant (“first MSP monomer domain variant”), and (2) determining whether the first MSP monomer domain variant specifically binds to the first target molecule. The first MSP monomer domain variant can be either a domain I variant or a domain II variant as described above. In certain variations, the first MSP monomer domain variant is from a polypeptide library as described above and the method includes screening the polypeptide library for binding to the first target molecule. In some such embodiments, the first MSP monomer domain variant is identified as capable of specifically binding to the target molecule and the method further includes (i) contacting the polypeptide library with a second target molecule; (ii) identifying a second MSP monomer domain variant that binds to the second target molecule; and (iii) linking the first and second MSP monomer domain variants to form a multimer comprising both the first and second MSP monomer domain variants. Such embodiments can further include (iv) contacting the multimer with the first and second target molecules; and (v) determining whether the multimer binds to both the first and second target molecules.

In some embodiments of the above method, the first MSP monomer domain variant is identified as capable of specifically binding to the target molecule. In some such embodiments, the method further includes linking the first MSP monomer domain variant to a second monomer domain to form a multimer comprising at least two monomer domains. The second monomer domain can be, for example, a second MSP monomer domain variant selected from a domain I variant and a domain II variant. Accordingly, in some variations in which monomer domains are linked to form a multimer, the multimer comprises both a domain I variant and a domain II variant. In particular embodiments, the domain II variant is linked C-terminal and directly adjacent to the domain I variant. In some such embodiments, the domain I variant comprises cysteine C₃₇ and the domain II variant comprises cysteine C₇₃.

In particular variations of the above method in which the first MSP monomer domain is linked to a second monomer domain, the second monomer domain is a monomer domain identified as capable of binding to a second target molecule. In some such embodiments, the method further includes contacting the multimer comprising the first and second monomer domains with the first and second target molecules; and determining whether the multimer binds to the first and second target molecules.

In other variations of the above method in which the first MSP monomer domain is linked to a second monomer domain, the second monomer domain is a monomer domain identified as capable of binding to the first target molecule. In some such embodiments, the method further includes contacting the multimer comprising the first and second monomer domains with the first target molecule; and determining whether the multimer binds to the first target molecule. Such embodiments of the method can further include determining whether the multimer has an increased binding affinity or avidity for the first target molecule relative to the first MSP monomer domain variant.

In some embodiments of the above method in which the first MSP monomer domain is linked to a second monomer domain, the method includes linking the first MSP monomer domain variant to a library of second monomer domains to form a library of multimers, where the library of multimers comprising a pool of multimers having different second monomer domains. Such embodiments may further include contacting the library of multimers with the first target molecule and identifying a multimer that binds to the first target molecule. In some such variations, the method further comprises determining whether the identified multimer binds to the first target molecule with greater affinity or avidity relative to the first MSP monomer domain variant.

Other aspects and variations of the present invention are further described herein.

### DETAILED DESCRIPTION OF THE INVENTION

### I. Overview

The present invention is based in part on the use of β-microseminoprotein (MSP) as an alternative scaffold or framework for polypeptide display and the development of novel binding molecules. MSP has the characteristics desired for an alternative scaffold: it is a small protein with a rigid backbone, contributed by the disulfide bonds, and has unstructured loops that are substantially variable across species lines. Although the amino acid sequence is highly variable among MSPs from different species, the ten cysteine residues contributing to the five disulfide bonds of MSP are completely conserved. (See, e.g., Lazure et al., Protein Sci. 10:2207-2218, 2001.) MSP is not glycosylated and has been made in E. coli and refolded to native structure as determined by NMR. (See Ghasriani et al., J. Mol. Biol 362:502-515, 2006.) The NMR solution structures for porcine and human MSPs have been determined and found to be superimposable. (See Ghasriani et al, supra.) The nucleotide coding and corresponding amino acid sequences for human MSP (including secretory signal sequence) are shown in SEQ ID NOs: 1 and 2, respectively. The nucleotide coding and amino acid sequences for the mature human MSP protein are shown in SEQ ID NOs: 3 and 4, respectively.

Based on NMR structure, MSP can be divided into two structural domains, or “monomer domains”: domain I, approximately corresponding to the 55 amino-terminal amino acids in a four- or six-stranded beta sheet, and domain II, approximately corresponding to the carboxy-terminal 39 amino acids in two, two-stranded beta sheets. In the full MSP protein, these domains are held together by hydrophobic, electrostatic interactions, and one disulfide bond between cysteines 37 and 73 (amino acid residue position numbers according to the mature human MSP protein as set forth in SEQ ID NO:4). In accordance with the present invention, the two individual monomer domains expressed separately, as well as the full protein, can serve as scaffolds for polypeptide display and for the development of novel binding molecules. For example, MSP and its monomer domains can be used as frameworks to construct polypeptide libraries that can be screened for polypeptides binding to desired targets. Further, multi-specific binders can be constructed by combining individual binding monomer domains into the full MSP structure or by tethering binding monomer domains together in, e.g., a “string of beads” configuration. Combined binders could be specific to epitopes on different target molecules or to different epitopes on the same target molecule. The combinatorial aspect of this concept allows for tuning the apparent affinity toward a target by changing avidity through addition or subtraction of monomer domain binding units.

The potential applications of MSP monomer domain variants and multimers of the present invention are diverse and include any use where an affinity agent is desired. For example, the invention can be used in the application for creating antagonists, where the selected MSP monomer domains or multimers block the interaction between two proteins (e.g., binding of a ligand/receptor or receptor/counter-receptor pairs). In other, alternative applications, the invention can generate agonists, such as, e.g., agonists of cell surface receptors or, for example, multimeric polypeptides binding two different proteins, e.g., enzyme and substrate, to enhance protein function, including, for example, enzymatic activity and/or substrate conversion. Other applications include cell targeting (e.g., MSP monomer domain variants that recognize specific cell surface proteins can bind selectively to certain cell types), or use as antiviral agents (e.g., multimers binding to different epitopes on the virus particle can be useful as antiviral agents because of the polyvalency). Still other applications include, but are not limited to, protein purification, protein detection, biosensors, ligand-affinity capture experiments, and the like. Furthermore, MSP monomer domains or multimers comprising one or more MSP domains can be synthesized in bulk by conventional means for any suitable use, such as, e.g., as a therapeutic or diagnostic agent.

These and other aspects of the invention will become evident upon reference to the following detailed description. In addition, various references are identified below and are incorporated by reference in their entirety.

### II. Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise.

The term “monomer domain” or “monomer” is used interchangeably herein refer to a discrete region found in a protein or polypeptide. A monomer domain forms a native three-dimensional structure in solution in the absence of flanking native amino acid sequences. Monomer domains include, for example, domains consisting of α-helices, small domains with few secondary structures or an irregular architecture of α-helices and α-sheets, and domains containing predominantly α-sheets. Monomer domains may further include stabilizing disulfide bonds linking spatially separated strands of the protein. Monomer domains can be selected to specifically bind to a target molecule. In addition to MSP monomer domains as described herein, exemplary “non-MSP” monomer domains for use as scaffolds include Z-domain of protein A; immunity proteins such as E. coli colicin E7 (ImmE7); cytochrome b₅₆₂; peptide α₂p8; repeat-motif proteins such as ankrin repeats; insect defensin A (IICA₂₉); kunitz domains such as BPTI/APPA; PDZ domains such as Ras-binding protein AF-6; scorpion toxins such as charybdotoxin; plant homeodomain (PHD) finger protein; TEM-1 b-lactamase; 10th fibronectin type III domain (¹⁰Fn3); the extracellular domain of CTLA-4; T-cell receptors; knottins such as Min-23 and cellulose binding domain; neocarzinostatin; CBM-2; tendamistat; lipocalins such as apolipoprotein D, bilin-binding protein, and FABP. (See generally, e.g., Hosse et al., Protein Sci. 15:14-27, 2006.)

“MSP monomer domain,” as used herein, refers to a monomer domain of a protein belonging to the β-microseminoprotein (MSP) family of proteins, including variants of naturally occurring MSP monomer domains (“MSP monomer domain variants” or “non-naturally occurring MSP monomer domain”). Generally, an MSP monomer domain will correspond to domain I or domain II of MSP as previously discussed, retaining conserved, paired cysteine residues and having an NMR structure substantially superimposable with a naturally occurring MSP monomer domain (e.g., substantially superimposable with domain I or domain II from a human or other mammalian MSP). Accordingly, an MSP monomer domain will generally correspond to amino acids 1-55 (domain I) or amino acids 54-94 (domain II) of human MSP (SEQ ID NO:4).

MSP monomer domains, including MSP monomer domain variants according to the present invention, are characterized in part by the presence of one or more loop sequences. The term “loop” generally refers to a portion of a monomer domain that is typically exposed to the environment by the assembly of the scaffold structure of the monomer domain protein, and that is involved in binding to a target molecule. MSP loop sequences of MSP domain I include “loop I” (corresponding approximately to amino acid resides 8-17 of SEQ ID NO:4), “loop II” (corresponding approximately to amino acid residues 43-47 of SEQ ID NO:4), and “loop V” (corresponding approximately to amino acid residues 21-25 of SEQ ID NO:4). MSP domain II includes the loop sequence of “loop III” (corresponding approximately to amino acid residues 57-63) and “loop IV” (corresponding approximately to amino acid residues 78-85 of SEQ ID NO:4).

The terms “MSP monomer domain variant” and “non-naturally-occurring MSP monomer domain” are used interchangeably herein to refer to a domain having an amino acid sequence different from that found in naturally occurring MSP proteins (e.g., different from MSP monomer domains of a human, primate, or other mammalian MSP protein.) MSP monomer domain variants can be obtained by human manipulation of an MSP monomer domain sequence. Examples of man-manipulated changes include, e.g., random mutagenesis, site-specific mutagenesis, recombining, directed evolution, oligo-directed forced crossover events, direct gene synthesis incorporation of mutation, and the like. Characteristic features of an MSP monomer domain variant include the substitution of a heterologous polypeptide segment at least one loop region (e.g., loop I, II, and/or V of MSP domain I; loop III and/or loop IV of MSP domain II), as well as conservation of paired cysteine residues within the respective monomer domain. Non-naturally occurring MSP monomer domains generally comprise an amino acid sequence having at least 60% sequence identity to a corresponding wild-type MSP polypeptide region, exclusive of any substitutions at the loop regions. In more typical variations, the MSP monomer domain variant will have at least 70%, at least 75%, at least 80%, at least 85%, at least 90%, or at least 95% sequence identity to a corresponding wild-type MSP polypeptide region, exclusive of any substitutions at the loop regions.

The term “unmodified form,” in the context of an MSP monomer domain variant, is a term used herein for purposes of defining the MSP monomer domain variant: the term “unmodified form” refers to an MSP monomer domain that has the amino acid sequence of the domain variant except at one or more amino acid position(s) or segment(s) specified as characterizing the domain variant, and except that the variant may further deviate from unmodified form sequence within a specified range of amino acid sequence identity for a particular region or regions of the variant domain (e.g., at least 60%, at least 70%, etc., at regions outside of any substituted loop regions). Thus, for example, reference to an MSP monomer domain variant in terms of (a) its unmodified form; (b) one or more substitutions at specified positions or segments; and (c) a specified degree of amino acid sequence identity means that, with the exception of the specified substitution(s), the MSP domain variant has an amino acid sequence having the specified degree of identity to that of the unmodified form. Reference of an MSP monomer domain variant in terms of its unmodified form may further specify certain amino acid positions that are absolutely conserved in the domain variant (e.g., conserved, paired cysteine residues). Accordingly, in carrying out the present invention as described herein, the unmodified form of an MSP monomer domain is predetermined. The unmodified form of an MSP monomer domain can be, for example, a wild-type and/or a naturally occurring MSP monomer domain.

In the context of MSP polypeptides, including, e.g., MSP monomer domains and full-length or substantially full-length MSP proteins, “correspondence” to another sequence (e.g., regions, fragments, nucleotide or amino acid positions, or the like) is based on the convention of numbering according to nucleotide or amino acid position number and then aligning the sequences in a manner that maximizes the percentage of sequence identity. Because not all positions within a given “corresponding region” need be identical, non-matching positions within a corresponding region may be regarded as “corresponding positions.” Accordingly, as used herein, referral to an “amino acid position corresponding to amino acid position [X]” of a specified MSP protein represents, in addition to referral to amino acid positions of the specified MSP protein, referral to a collection of equivalent positions in other recognized MSP proteins and structural homologues and families. In typical embodiments of the present invention, “correspondence” of amino acid positions are specified with reference to the human MSP protein of SEQ ID NO:4.

The use herein of braces (“{ }”) in sequence motifs indicates the presence of a plurality of amino acids. For example, “{Z}” indicates the presence of a sequence of amino acids constituting loop I of an wild-type MSP protein, or heterologous loop sequence in an MSP domain I variant; while “{x₈₈₋₉₄}” represents a sequence of seven amino acids corresponding to residues 88 to 94 of the MSP protein of SEQ ID NO:4. The use herein of square brackets (“[ ]”) in sequence motifs indicates alternate possible amino acids within a position (e.g., “[ndg]” indicates that either N, D, or G may be at that position). The use herein of parentheses in a motif indicates that that the positions within the parentheses may be present or absent (e.g., “(x) indicates that the position is absent or present; “([tn])” indicates that the position is absent or either T or N may be at that position). When more than one “x” is used in parentheses (e.g., “(xx)”), each x represents a possible position. Thus, for example, “(xx)” indicates that zero, one, or two amino acids may be at that position(s); “({x₈₈₋₉₄})” indicates that none, or one or more, of the amino acids corresponding to residues 88 to 94 of the MSP protein of SEQ ID NO:4, may be present.

The term “naturally occurring,” in the context of MSP polypeptides and nucleic acids, means an MSP polypeptide or nucleic acid having an amino acid or nucleotide sequence that is found in nature, i.e., an amino acid or nucleotide sequence that can be isolated from a source in nature (an organism) and which has not been intentionally modified by human intervention.

As used herein, “wild-type MSP gene” or “wild-type MSP nucleic acid” refers to a sequence of nucleic acid, corresponding to a MSP genetic locus in the genome of an organism, that encodes a gene product having an amino acid sequence, corresponding to the genetic locus, that is most commonly found in the natural population of the species of organism (the “most frequent amino acid sequence corresponding to the genetic locus”). A wild-type MSP gene may, for example, comprise any naturally-occurring nucleotide sequence encoding the gene product having the most frequent amino acid sequence corresponding to the genetic locus. In addition, due to the degeneracy of the genetic code, wild-type MSP genes may comprise other, non-naturally-occurring nucleotide sequences encoding the most frequent amino acid sequence corresponding to the genetic locus.

The term “wild-type MSP polypeptide” or “wild-type MSP protein” refers to an MSP polypeptide encoded by a wild-type MSP gene.

A polynucleotide or amino acid sequence is “heterologous to” a second sequence if the two sequences are not linked in the same manner as found in naturally-occurring sequences. For example, a promoter operably linked to a heterologous coding sequence refers to a coding sequence which is different from any naturally-occurring allelic variants. The term “heterologous linker,” when used in reference to a multimer, indicates that the multimer comprises a linker and a monomer that are not found in the same relationship to each other in nature (e.g., they form a fusion protein).

The term “heterologous,” in particular reference to a polypeptide segment at a modified MSP loop region, means that the polypeptide segment has one or more amino acid substitutions, additions, or deletions relative to the corresponding unmodified loop region (i.e., relative to the loop region of the wild-type MSP polypeptide from which an MSP variant is derived).

The term “multimer” is used herein to indicate a polypeptide comprising at least two monomer domains, or comprising at least one monomer domain and at least one antigen-binding domain. Multimers of the present invention generally include at least one MSP monomer domain variant as described herein. The separate monomer domains and/or antigen-binding domains in a multimer can be joined together by a linker. Individual monomer domains or antigen-binding domains of a multimer are also interchangeably referred to herein as “domains” or “subunits” of the multimer.

The term “target” or “target molecule” means a molecule that is to be used in a screening assay for determination of binding capability to a polypeptide comprising one or more monomer domains. The term encompasses a wide variety of molecules, which range from simple molecules to complex targets. Target molecules can be proteins, nucleic acids, lipids, carbohydrates or any other molecule potentially capable of recognition by a polypeptide domain (e.g., capable of recognition by an MSP monomer domain).

As used herein, the term “antigen-binding domain” refers to a protein binding domain that contains at least one complementarity determining region (CDR) of an antibody. The term “antibody” is used herein to denote a protein produced by the body in response to the presence of an antigen and that binds to the antigen, as well as antigen-binding fragments and engineered variants thereof. Thus, the term “antibody” is used expansively to include any protein that comprises an antigen-binding site (“antigen-binding domain”) of an antibody and is capable of binding to its antigen. Accordingly, antigen-binding domains can be naturally occurring antigen-binding domains (i.e., isolated from nature) or can be non-naturally occurring antigen-binding domains that have been altered by human-manipulation (e.g., via mutagenesis methods, such as, for example, random mutagenesis, site-specific mutagenesis, recombination, and the like, as well as by directed evolution methods, such as, for example, recursive error-prone PCR, recursive recombination, and the like). Examples of antigen-binding domains, and which are particularly suitable for use in accordance with the present invention, include single-domain antibodies, minibodies, Fv fragments, single-chain Fv fragments (scFv), and Fab fragments, to name a few.

The term “minibody” refers herein to a polypeptide that encodes only 2 complementarity determining regions (CDRs) of a naturally or non-naturally (e.g., mutagenized) occurring heavy chain variable domain or light chain variable domain, or combination thereof. Examples of minibodies are described by, e.g., Pessi et al., Nature 362:367-369, 1993; and Qiu et al., Nature Biotechnol. 25:921-929, 2007.

The term “single-domain antibody,” as used herein, refers to the heavy chain variable domain (“V_{H}”) of an antibody, i.e., a heavy chain variable domain without a light chain variable domain. Exemplary single-domain antibodies employed in the practice of the present invention include, for example, the Camelid heavy chain variable domain (about 118 to 136 amino acid residues) as described by Hamers-Casterman et al., Nature 363:446-448, 1993) and Dumoulin et al. (Protein Sci. 11:500-515, 2002).

An immunoglobulin “Fv” fragment contains a heavy chain variable domain (V_{H}) and a light chain variable domain (V_{L}), which are held together by non-covalent interactions. The dimeric structure of an Fv fragment can be further stabilized by the introduction of a disulfide bond via mutagenesis. (See Almog et al., Proteins 31:128-138, 1998.)

As used herein, the terms “single-chain Fv” and “single-chain antibody” refer to antibody fragments that comprise, within a single polypeptide chain, the variable regions from both heavy and light chains, but lack constant regions. In general, a single-chain antibody further comprises a polypeptide linker between the V_{H} and V_{L} domains, which enables it to form the desired structure that allows for antigen binding. Single-chain antibodies are discussed in detail by, for example, Pluckthun in The Pharmacology of Monoclonal Antibodies, vol. 113 (Rosenburg and Moore eds., Springer-Verlag, New York, 1994), pp. 269-315. (See also WIPO Publication WO 88/01649; U.S. Pat. Nos. 4,946,778 and 5,260,203; Bird et al, Science 242:423-426, 1988.) Single-chain antibodies can also be bi-specific and/or humanized.

As used herein, the term “Fab fragment” refers to an antibody fragment that has two protein chains, one of which is a light chain consisting of two light chain domains (V_{L} variable domain and C_{L} constant domain) and a heavy chain consisting of two heavy domains (V_{H} variable domain and C_{H} constant domain). Fab fragments employed in the practice of the present invention include those that have an interchain disulfide bond at the C-terminus of each heavy and light component, as well as those that do not have such a C-terminal disulfide bond. Each fragment is about 47 kD. Fab fragments are described by, e.g., Pluckthun and Skerra (Methods Enzymol 178:497-515, 1989).

The term “linker” is used herein to indicate a moiety or group of moieties that joins or connects two or more discrete, separate monomer domains. The linker allows the discrete, separate monomer domains to remain separate when joined together in a multimer. The linker moiety is typically a substantially linear moiety. Suitable linkers include polypeptides, polynucleic acids, peptide nucleic acids, and the like. Suitable linkers also include optionally substituted alkylene moieties that have one or more oxygen atoms incorporated in the carbon backbone. Typically, the molecular weight of the linker is less than about 2000 daltons. More typically, the molecular weight of the linker is less than about 1500 daltons and usually is less than about 1000 daltons. The linker can be small enough to allow the discrete, separate monomer domains to cooperate, e.g., where each of the discrete, separate monomer domains in a multimer binds to the same target molecule via separate binding sites. Exemplary linkers include a polynucleotide encoding a polypeptide, or a polypeptide of amino acids or other non-naturally occurring moieties. The linker can be a portion of a native sequence, a variant thereof, or a synthetic sequence. Linkers can comprise, e.g., naturally occurring amino acids, non-naturally occurring amino acids, or a combination of both.

The term “separate,” in the particular context of multiple moieties (e.g., monomer domains, antigen-binding sites of an antibody) is used herein to indicate a property of a moiety that is independent and remains independent even when complexed with other moieties. For example, a monomer domain is a separate domain in a protein because it has an independent property that can be recognized and separated from the protein (e.g., domain I or domain II of an MSP protein). Monomer domains in a multimer are separate where they remain separate, independent domains even when complexed or joined together in the multimer by a linker.

The term “adjacent,” in reference to two, linked polypeptide segments, means that the polypeptide segments are non-overlapping and not separated by an intervening segment (e.g., linker).

As used herein, “directed evolution” refers to a process by which polynucleotide variants are generated, expressed, and encoded polypeptides screened for an activity (e.g., binding activity) in a recursive process. One or more candidates in the screen are selected and the process is then repeated using polynucleotides that encode the selected candidates to generate new variants. Directed evolution involves at least two rounds of variation generation and can include 3, 4, 5, 10, 20, or more rounds of variation generation and selection. Variation can be generated by any method known to those of skill in the art, including, e.g., by error-prone PCR, gene recombination, chemical mutagenesis, and the like.

The term “shuffling” is used herein to indicate recombination between non-identical sequences. In some embodiments, shuffling can include crossover via homologous recombination or via non-homologous recombination, such as via cre/10× and/or flp/frt systems. Shuffling can be carried out by employing a variety of different formats, including for example, in vitro and in vivo shuffling formats, in silico shuffling formats, shuffling formats that utilize either double-stranded or single-stranded templates, primer based shuffling formats, nucleic acid fragmentation-based shuffling formats, and oligonucleotide-mediated shuffling formats, all of which are based on recombination events between non-identical sequences and are described in more detail or referenced herein below, as well as other similar recombination-based formats.

The term “random” as used herein refers to a polynucleotide sequence or an amino acid sequence composed of two or more amino acids and constructed by a stochastic or random process. The random polynucleotide sequence or amino acid sequence can include framework or scaffolding motifs, which can comprise invariant sequences (e.g., an MSP domain framework or scaffold as described further herein).

The term “pseudorandom” as used herein refers to a set of polynucleotide or polypeptide sequences that have limited variability, so that the degree of residue variability at one or more positions is limited, but any pseudorandom position is allowed at least some degree of residue variation.

The term “semi-random” as used herein refers to a set of polynucleotide or polypeptide sequences that have limited variability, so that one or more, but not all, positions are fixed, while other positions are randomized or pseudorandomized.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

A “polypeptide” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

A “cloning vector” is a nucleic acid molecule, such as a plasmid, cosmid, or bacteriophage that has the capability of replicating autonomously in a host cell. Cloning vectors typically contain one or a small number of restriction endonuclease recognition sites that allow insertion of a nucleic acid molecule in a determinable fashion without loss of an essential biological function of the vector, as well as nucleotide sequences encoding a marker gene that is suitable for use in the identification and selection of cells transformed with the cloning vector. Marker genes typically include genes that provide tetracycline resistance or ampicillin resistance.

An “expression vector” is a nucleic acid molecule encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter.

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In certain embodiments, the two sequences are the same length.

The term “substantially identical,” in the context of two nucleic acids or polypeptides, refers to two or more sequences or subsequences that have at least 50%, at least 55%, at least 60%, or at least 65% identity; typically at least 70% or at least 75% identity; more typically at least 80%, at least 85%, at least 90%, or at least 95% identity.

“Similarity” or “percent similarity” in the context of two or more polypeptide sequences, refer to two or more sequences or subsequences that have a specified percentage of amino acid residues that are the same or conservatively substituted when compared and aligned for maximum correspondence. By way of example, a first amino acid sequence can be considered similar to a second amino acid sequence when the first amino acid sequence is at least 50%, 60%, 70%, 75%, 80%, 90%, or even 95% identical, or conservatively substituted, to the second amino acid sequence when compared to an equal number of amino acids as the number contained in the first sequence, or when compared to an alignment of polypeptides that has been aligned by a computer similarity program known in the art (see infra).

The term “substantial similarity,” in the context of polypeptide sequences, indicates that a polypeptide region has a sequence with at least 70% or at least 75%, typically at least 80% or at least 85%, and more typically at least 85%, at least 90%, or at least 95% sequence similarity to a reference sequence. For example, a polypeptide is substantially similar to a second polypeptide, for example, where the two peptides differ by one or more conservative substitutions.

The determination of percent identity or percent similarity between two sequences can be accomplished using a mathematical algorithm. A preferred, non-limiting example of a mathematical algorithm utilized for the comparison of two sequences is the algorithm of Karlin and Altschul (Proc. Natl. Acad. Sci. USA 87:2264-2268, 1990), modified as in Karlin and Altschul (Proc. Natl. Acad. Sci. USA 90:5873-5877, 1993). Such an algorithm is incorporated into the NBLAST and XBLAST programs of Altschul et al. (J Mol. Biol. 215:403-410, 1990). BLAST nucleotide searches can be performed with the NBLAST program, score=100, wordlength=12 to obtain nucleotide sequences homologous to a nucleic acid encoding a protein of interest. BLAST protein searches can be performed with the XBLAST program, score=50, wordlength=3 to obtain amino acid sequences homologous to protein of interest. To obtain gapped alignments for comparison purposes, Gapped BLAST can be utilized as described in Altschul et al. (Nucleic Acids Res. 25:3389-3402, 1997). Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. (See, e.g., the National Center for Biotechnology Information (NCBI) website, www.ncbi.nlm.nih.gov.) Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (Comput. Appl. Biosci. 10:3-5, 1994); and FASTA described in Pearson and Lipman (Proc. Natl. Acad. Sci. USA 85:2444-8, 1988). Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see, e.g., bioweb.pasteur.fr/docs/man/man/fasta.1.html#sect2, the contents of which are incorporated herein by reference.

Alternatively, protein sequence alignment may be carried out using the CLUSTAL W algorithm, as described by Higgins et al. (Methods Enzymol. 266:383-402, 1996).

Due to the imprecision of standard analytical methods, molecular weights and lengths of polymers are understood to be approximate values. When such a value is expressed as “about” X or “approximately” X, the stated value of X will be understood to be accurate to ±10%.

### III. MSP Monomer Domain Variants and Multimers Thereof

In one aspect, the present invention provides MSP monomer domain variants as well as multimers comprising one or more such MSP monomer domain variants. In accordance with certain aspects of the invention, the MSP monomer domain variants and multimers thereof serve as affinity agents for binding to target molecules. For example, in certain variations, the MSP monomer domains and multimers can be selected for the ability to bind to a desired target molecule or mixture of target molecules. In particular embodiments, multimers comprising an MSP monomer domain variant having specificity for a target molecule can be screened to identify those multimers that have an improved characteristic such as, e.g., improved avidity or affinity or altered specificity for the target or the mixture of targets, compared to the monomer domain alone.

Polypeptides of the invention generally comprise at least one MSP monomer domain variant corresponding to domain I or domain II of a wild-type MSP protein. In particular, the variants include one or more loop regions that have been modified relative to the corresponding wild-type MSP protein. Particularly suitable loop regions for modification include the polypeptide regions corresponding to amino acid residues 8-17 (“loop I”), 43-47 (“loop II”), 57-63 (“loop III”), 78-85 (“loop IV”), and 21-25 (“loop V”) of human MSP (SEQ ID NO:4), where loops I, II, and V are within domain I and loops III and IV are within domain II of MSP. Modifications to one or more of these loop regions can include one or more amino acid substitutions, additions, and/or deletions relative to the wild-type sequence, with the modified loop region typically having at least four and up to about 50 or about 100 amino acids, and more typically at least five or six amino acids and up to about 25 or 30 amino acids, resulting in a loop sequence that is different from (“heterologous to”) the wild-type loop region. As further described herein, these heterologous loop sequences can be, for example, randomized, pseudorandomized, or semi-randomized among different polypeptides to generate diversity among MSP monomer domain variants that can be screened for certain characteristics (e.g., target binding).

In certain embodiments in which a polypeptide comprises an MSP monomer domain I variant, the unmodified form of the MSP domain I includes the amino acid sequence (x₁)C₂{x₃₋₇}{Z_{I}}C₁₈{x₁₉₋₃₆}C₃₇x₃₈x₃₉C₄₀x₄₁C₄₂{Z_{II}}x₄₈C₄₉C₅₀(x₅₁₋₅₅) (SEQ ID NO:59), which sequence represents a polypeptide region from a wild-type MSP protein corresponding to amino acid residues 1 to 55 of human MSP (SEQ ID NO:4), and where {Z_{I}} and {Z_{II}} denote loop regions corresponding, respectively, to amino acids 8-17 and 43-47 of human MSP. In its modified form, the MSP monomer domain I variant conserves each of cysteine residues C₂, C₁₈, C₄₀, C₄₂, C₄₉, and C₅₀; and also includes a modified {Z_{I}} and/or a modified {Z_{II}} loop region. Typically, the modified loop region is a heterologous polypeptide segment of from 3 or 4 amino acids to about 100 amino acids; more typically from 3 or 4 amino acids to about 50 amino acids; and still more typically from 4 amino acids to about 30 amino acids, from 5 amino acids to about 30 amino acids, or from 6 amino acids to about 25 amino acids. In specific variations, a modified {Z_{I}} loop region is a heterologous polypeptide segment of 10 amino acids and a modified {Z_{II}} loop region is a heterologous polypeptide segment of 5 amino acids. In some embodiments, a monomer domain I variant includes both the modified {Z_{I}} and the modified {Z_{II}} loop regions; alternatively, only one of {Z_{I}} and {Z_{II}} is modified relative to the wild-type MSP sequence. In addition to modification at one or more of the loop regions, the domain I variant may comprise an amino acid sequence that is at least 60% identical to the wild-type MSP polypeptide region, typically at least 70% or 75% identical, and more typically at least 80%, at least 85%, at least 90%, or at least 95% identical, such identity being exclusive of any modifications at {Z_{I}} and/or {Z_{II}}. In some variations, a non-sulfhydryl-containing amino acid residue is substituted for C₃₇.

In some variations of an MSP monomer domain I variant, the domain I variant is substantially as described above, but with a modified loop V region (between β2 and β3; amino acids corresponding to residues 21-25 of SEQ ID NO:4) in addition to or in lieu of one or both of the modified {Z_{I}} and {Z_{II}} loop regions. Typically, the modified loop V region {Z_{V}} is a heterologous polypeptide segment of from 3 or 4 amino acids to about 100 amino acids; more typically from 3 or 4 amino acids to about 50 amino acids; and still more typically from 4 amino acids to about 30 amino acids, from 5 amino acids to about 30 amino acids, or from 6 amino acids to about 25 amino acids. In specific variations, a modified {Z_{V}} loop region is a heterologous polypeptide segment of 5 amino acids. In some embodiments, a monomer domain I variant includes only the modified {Z_{V}} loop region, with both the {Z_{I}} and {Z_{II}} loop regions maintaining the wild-type MSP sequence. In alternative embodiments, the domain I variant includes the modified {Z_{V}} loop region together with one or both of {Z_{I}} and {Z_{II}} being modified relative to the wild-type MSP sequence. For example, in a specific variation, the domain I variant includes both the modified {Z_{I}} and {Z_{V}} loop regions, with the wild-type MSP sequence being maintained at loop II ({Z_{II}}). In addition to modification at one or more of the loop regions, the domain I variant may comprise an amino acid sequence that is at least 60% identical to the wild-type MSP polypeptide region, typically at least 70% or 75% identical, and more typically at least 80%, at least 85%, at least 90%, or at least 95% identical, such identity being exclusive of any modifications at {Z_{I}}, {Z_{II}}, and/or {Z_{V}}. In some variations, a non-sulfhydryl-containing amino acid residue is substituted for C₃₇.

In other embodiments in which a polypeptide comprises an MSP monomer domain II variant, the unmodified form of the MSP domain II includes the amino acid sequence {x₅₄₋₅₆}{Z_{III}}C₆₄{x₆₅₋₇₂}C₇₃{x₇₄₋₇₇}{Z}x₈₆C₈₇({x₈₈₋₉₄}) (SEQ ID NO:60), which sequence represents a polypeptide region from a wild-type MSP protein corresponding to amino acid residues 52 to 94 of human MSP (SEQ ID NO:4), and where {Z_{III}} and {Z_{IV}} denote loop regions corresponding, respectively, to amino acids 57-63 and 78-85 of human MSP. In its modified form, the MSP monomer domain I variant conserves each of cysteine residues C₆₄ and C₈₇; and also includes a modified {Z_{III}} and/or a modified {Z_{IV}} loop region. Typically, the modified loop region is a heterologous polypeptide segment of from 3 or 4 amino acids to about 100 amino acids; more typically from 3 or 4 amino acids to about 50 amino acids; and still more typically from 4 amino acids to about 30 amino acids, from 5 amino acids to about 30 amino acids, or from 6 amino acids to about 25 amino acids. In specific variations, a modified {Z_{III}} loop region is a heterologous polypeptide segment of 7 amino acids and a modified {Z_{IV}} loop region is a heterologous polypeptide segment of 8 amino acids. In some embodiments, a monomer domain II variant includes both the modified {Z_{III}} and the modified {Z_{IV}} loop regions; alternatively, one of {Z_{III}} and {Z_{IV}} is modified relative to the wild-type MSP sequence. In addition to modification at one or more of the loop regions, domain II variants may comprise an amino acid sequence that is at least 60% identical to the wild-type MSP polypeptide region, typically at least 70% or 75% identical, and more typically at least 80%, at least 85%, at least 90%, or at least 95% identical, such identity being exclusive of any modifications at {Z_{III}} and/or {Z_{IV}}. In some variations, a non-sulfhydryl-containing amino acid residue is substituted for C₇₃.

The wild-type MSP protein from which an MSP monomer domain variant is derived can be, for example, a mammalian, avian, amphibian, or fish MSP protein. Exemplary wild-type avian MSPs include those from chicken and ostrich, while an exemplary wild-type amphibian MSP includes Xenopus MSP. Wild-type MSP proteins from fish include, for example, MSPs from zebrafish and flounder, while examples of wild-type mammalian MSP proteins include MSPs from artiodactyls (e.g., cow and porcine), rodents (e.g., rat and mouse), and primates (e.g., human, apes, and monkeys). Amino acid sequences for MSP proteins of various animal species are known, including MSPs for human (Homo sapiens; accession No. AJ13356; Mbikay et al, DNA 6:23-29, 1987); rhesus monkey (Macacca mulatta; accession No. M92161; Nolet et al, Genomics 9:775-777, 1991); baboon (Papio hamadryas anubis; accession No. U49786; Xuan et. al., DNA Cell Biol 16:627-638, 1997); cotton-top tamarin (Saguinus oedipus; accession Nos. mspE1, AJ010154; mspA1, AJ010158; and mspJ1, AJ010156; Mäkinen et al., Eur. J. Biochem. 264:407-414, 1999); porcine (Sus scrofa; accession No. S41663; Femlund et al., Arch. Biochem. Biophys. 309:70-76, 1994; Tanaka et al., Mol. Reprod. Dev. 42:149-156, 1995); rat (Rattus norvegicus; accession No. U65486; Femlund et al., Arch. Biochem. Biophys. 334:73-82, 1996); murine (Mus musculus; accession No. J89840; Xuan et al., DNA Cell Biol. 18:11-26, 1999); ostrich (Struthio camelus; Lazure et al., Protein Science 10:2207-2218, 2001); chicken (Gallus gallus; Warr, Dev. Comp. Immunol 14:247-253, 1990); African clawed frog (Xenopus laevis; Lazure et al., supra [back-translating from cDNA accession No. AW641318]); zebrafish (Danio rerio; Lazure et al., supra [back-translating from cDNA accession No. AI497271]); and two Japanese flounder sequences (Paralichthys olivaceus; Lazure et al., supra [back-translating from cDNA accession Nos. C23089 and C23023]). In certain embodiments, an MSP monomer domain is a variant of domain I or II of the wild-type human MSP protein of SEQ ID NO:4.

For example, in specific embodiments of human MSP-derived variants, the domain variants conserve the wild-type human MSP sequence outside of any modified loop region(s), except that unpaired cysteines of domain I or domain II (C₃₇ or C₇₃) may be substituted with a non-sulfhydryl residue. Such variants generally comprise the sequence (S)C₂YFIPN{Z_{I}}C₁₈MDLKGNKHPINSEWQ TDNx₃₇ETC₄₀TC₄₂{Z_{II}}SC₄₉C₅₀(TLVST) (SEQ ID NO:48) or STP {Z_{III}}C₆₄QRIFKKEDx₇₃KYIV {Z_{IV}}TC₈₇(SVSEWII) (SEQ ID NO:49), where SEQ ID NO:48 and SEQ ID NO:49 represent, respectively, domain I and domain II variants derived from human MSP; where each of x₃₇ and x₇₃ is any amino acid; and where {Z_{I}}, {Z_{II}}, {Z_{III}}, and {Z_{IV}} denote loop regions corresponding, respectively, to amino acids 8-17, 43-47, 57-63, and 78-85 of human MSP, with at least one of {Z_{I}} and {Z_{II}} of domain I, or at least one of {Z_{III}} and {Z_{IV}} of domain II, being a heterologous polypeptide segment (i.e., a modified {Z_{I}}, {Z_{II}}, {Z_{III}}, and/or {Z_{IV}} loop region), as noted supra. In some embodiments, x₃₇ of the domain I variant and/or x₇₃ of the domain II variant is a non-sulfhydryl-containing amino acid residue, while in other embodiments (e.g., a polypeptide comprising both domain I and II variants), x₃₇ and x₇₃ may be cysteine (thereby conserving C₃₇ and C₇₃ of the wild-type human MSP protein). In certain variations, a domain 1 variant as above comprises an amino acid sequence as set forth in SEQ ID NO:79, SEQ ID NO:80, SEQ ID NO:91, or SEQ ID NO:35, and a domain II variant as above comprises an amino acid sequence as set forth in SEQ ID NO:87, SEQ ID NO:88, SEQ ID NO:92, or SEQ ID NO:37.

In other examples of MSP monomer domain variants derived from human MSP, a domain variant will comprise a sequence as described above for SEQ ID NO:48 or SEQ ID NO:49, but will include additional modifications outside of any modified loop regions or unpaired cysteine residue substitutions, such that the domain variant has an amino acid sequence at least 60% identical to the corresponding wild-type human MSP polypeptide region from SEQ ID NO:4, typically at least 70% or 75% identical, and more typically at least 80%, at least 85%, at least 90%, or at least 95% identical, such identity being exclusive of any modifications at {Z_{I}}, {Z_{II}}, {Z_{III}}, {Z_{IV}}, and/or {Z_{V}}. In more specific embodiments, such variants will include one, two, three, four, five, six, seven, or eight single amino acid substitutions, additions, or deletions relative to the corresponding wild-type human MSP sequence from SEQ ID NO:4, exclusive of any modifications at {Z_{I}}, {Z_{II}}, {Z_{III}}, {Z_{IV}}, and/or {Z_{V}} and further not including any substitution at C₃₇ or C₇₃.

Modifications to a wild-type MSP sequence that are outside a loop region ({Z_{I}}, {Z_{II}}, {Z_{III}}, {Z_{IV}}, {Z_{V}}) can include non-conservative as well as conservative amino acid changes. As previously noted, the primary structure of MSP shows a remarkably low level of conservation in amino acids among the species studied, and divergent MSP proteins have been shown to have superimposable NMR structures. Thus, the three-dimensional structure of MSP proteins and its domains is remarkably robust, and it is anticipated that any given wild-type MSP sequence will tolerate a variety of amino acid substitutions, additions, or deletions without comprising the three-dimensional structure of the protein, including the presentation of constrained polypeptide segments at one or more of the loop regions I, II, III, IV, and V.

For example, in certain embodiments, the present invention includes polypeptides having one or more conservative amino acid changes as compared with the amino acid sequence of a wild-type MSP protein (e.g., as compared with the amino acid sequence of the human MSP protein of SEQ ID NO:4). The BLOSUM62 matrix (Table 1) is an amino acid substitution matrix derived from about 2,000 local multiple alignments of protein sequence segments, representing highly conserved regions of more than 500 groups of related proteins (Henikoff and Henikoff, Proc. Natl. Acad. Sci. USA 89:10915-10919, 1992). Thus, the BLOSUM62 substitution frequencies can be used to define conservative amino acid substitutions that may be introduced into the amino acid sequences of the present invention. As used herein, the term “conservative amino acid substitution” refers to a substitution represented by a BLOSUM62 value of greater than 1. For example, an amino acid substitution is conservative if the substitution is characterized by a BLOSUM62 value of 0, 1, 2, or 3. Preferred conservative amino acid substitutions are characterized by a BLOSUM62 value of at least one 1 (e.g., 1, 2 or 3), while more preferred conservative amino acid substitutions are characterized by a BLOSUM62 value of at least 2 (e.g., 2 or 3).

TABLE 1
BLOSUM62 Amino Acid Substitution Matrix

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | |

Alternatively or in addition, a particularly suitable technique for guiding identification of suitable amino acid substitutions, additions, or deletions that may be introduced into a given wild-type sequence includes aligning MSP proteins by sequence alignment. There are a number of sequence alignment methodologies discussed above that may be used. Sequence-based alignment programs include, for example, Smith-Waterman searches, Needleman-Wunsch, Double Affine Smith-Waterman, frame search, Gribskov/GCG profile search, Gribskov/GCG profile scan, profile frame search, Bucher generalized profiles, Hidden Markov models, Hframe, Double Frame, Blast, Psi-Blast, Clustal, and GeneWise. (See, e.g., Altschul et al., J. Mol. Biol. 215:403-410, 1990; Altschul et al., Nucleic Acids Res. 25:3389-3402, 1997.)

The amino acid sequences of MSP proteins can be aligned, for example, into a multiple sequence alignment (MSA). Alignment of various MSP sequences can be readily carried out using publicly available MSP sequence information, and examples of such alignments are shown, e.g., by Wang et al. (J. Mol. Biol. 346:1071-1082, 2004) and Lazure et al. (supra). Due to the high extent of structural homology between different MSP proteins, the MSA can be used as a reliable predictor of the effects of modifications at various positions within the alignment. Using an MSA, and/or using alignment programs known in the art such as those described herein, one can use as a reference point the numbering system of the alignment program and may correlate the relevant positions of the MSP protein with equivalent positions in other recognized MSP family members. Similar methods can be used for alignment of the amino acid sequence(s) of MSP proteins that have yet to be sequenced.

For example, based on sequence alignment information, it is anticipated that one or more of the amino acid changes shown in Table 2 may be introduced into the wild-type human MSP sequence of SEQ ID NO:4 at regions outside of loops I, II, III, and IV.

TABLE 2
Exemplary Amino Acid Changes in Human MSP Domain
Variants Based on Cross-species MSP Sequence Alignment

| Amino Acid & | Exemplary |
|---|---|
| Position No. | Amino Acid |
| (SEQ ID NO: 4) | Substitution(s) |
| S1 | Q, A, V, Y, F, D |
| Y3 | S, F |
| F4 | L, I, Q, T, V, S |
| I5 | M, Q, E, K |
| P6 | R, N, I, L |
| N7 | L, R, P, H |
| M19 | T, Q, I, L, E |
| D20 | L, Y, I, M |
| L21 | V, A, Y, H, D |
| K22 | D |
| G23 | D, Q |
| N24 | V, G, K, T |
| K25 | S, L, W, R |
| H26 | Y |
| P27 | V, F, R, E |
| I28 | L, F, V |
| N29 | D, G |
| S30 | T, E |
| E31 | K, R, V, Y, P, I, S, T |
| W32 | S, P, F |
| Q33 | K, R, I |
| T34 | N |
| D35 | E, K, N, S |
| N36 | D, R, K |
| E38 | D, T, Y, M, F, L |
| T39 | A, R, E, S, L, W, D |
| T41 | D, A, F, S, I |
| S48 | I, T, R, A, H, E |
| T51 | S, N, Q, A, D |
| L52 | K, N, T, A |
| V53 | I, A, T, F, Y, M |
| S54 | A, L, T, H, G |
| T55 | I, R |
| Q65 | R, D, K, I, V, T |
| R66 | K, V, A, S |
| I67 | Q, V, K |
| F68 | L, Y |
| K69 | N, H, D |
| K70 | Q, S, P, R |
| E71 | K, S, Y |
| D72 | E, T, N, A, L, S |
| K74 | R, T, N, H, D, V |
| Y75 | I, N, F |
| I76 | S, T, D, R, E |
| T86 | E, P, L, K |
| S88 | P, E, G, D, R, F |
| V89 | I, G, Y |
| S90 | D, T, N, L, Y, G |
| E91 | Q, A, G, S, Y |
| W92 | R, M, A |
| I93 | V, T |
| I94 | L, M, S, G |

In some variations, MSP monomer domain variants will comprise certain amino acids that are highly conserved across species of diverse families. Accordingly, in some embodiments, an MSP monomer domain variant will include one or more of the following amino acids at the specified positions:

Aspartate (D) at the position corresponding to residue 20 of SEQ ID NO:4;

Glycine (G) at the position corresponding to residue 23 of SEQ ID NO:4;

Histidine (H) at the position corresponding to residue 26 of SEQ ID NO:4;

Tryptophan (W) at the position corresponding to residue 32 of SEQ ID NO:4;

Phenylalanine (F) at the position corresponding to residue 68 of SEQ ID NO:4;

Valine (V) at the position corresponding to residue 77 of SEQ ID NO:4; and

Valine (V) at the position corresponding to residue 78 of SEQ ID NO:4.

For example, in certain embodiments, an MSP monomer domain I variant will include D, G, H, and W at positions corresponding, respectively, to residues 20, 23, 26, and 32 of SEQ ID NO:4; and an MSP monomer domain II variant will include F, V, and V at positions corresponding, respectively, to residues 68, 77, and 78 of SEQ ID NO:4.

In yet other variation, MSP monomer domain variants will comprise certain amino acids that are highly conserved across primate species. Accordingly, in some embodiments, an MSP monomer domain variant will include one or more of the following amino acids at the specified positions:

Serine (S) at the position corresponding to residue 1 of SEQ ID NO:4;

Aspartate (D) at the position corresponding to residue 20 of SEQ ID NO:4;

Leucine (L) at the position corresponding to residue 21 of SEQ ID NO:4;

Lysine (K) at the position corresponding to residue 22 of SEQ ID NO:4;

Glycine (G) at the position corresponding to residue 23 of SEQ ID NO:4;

Asparagine (N) at the position corresponding to residue 24 of SEQ ID NO:4;

Lysine (K) at the position corresponding to residue 25 of SEQ ID NO:4;

Histidine (H) at the position corresponding to residue 26 of SEQ ID NO:4;

Proline (P) at the position corresponding to residue 27 of SEQ ID NO:4;

Serine (S) at the position corresponding to residue 30 of SEQ ID NO:4;

Tryptophan (W) at the position corresponding to residue 32 of SEQ ID NO:4;

Threonine (T) at the position corresponding to residue 34 of SEQ ID NO:4;

Leucine (L) at the position corresponding to residue 52 of SEQ ID NO:4;

Isoleucine (I) at the position corresponding to residue 67 of SEQ ID NO:4;

Phenylalanine (F) at the position corresponding to residue 68 of SEQ ID NO:4;

Glutamate (E) at the position corresponding to residue 71 of SEQ ID NO:4;

Valine (V) at the position corresponding to residue 77 of SEQ ID NO:4;

Valine (V) at the position corresponding to residue 78 of SEQ ID NO:4;

Tryptophan (W) at the position corresponding to residue 92 of SEQ ID NO:4; and

Isoleucine (I) at the position corresponding to residue 93 of SEQ ID NO:4.

For example, in certain embodiments, an MSP monomer domain I variant will include S, D, L, K, G, N, K, H, P, S, W, and T at positions corresponding, respectively, to residues 1, 20, 21, 22, 23, 24, 25, 26, 27, 30, 32, and 34 of SEQ ID NO:4; and an MSP monomer domain II variant will include I, F, E, V, V, W, and I at positions corresponding, respectively, to residues 67, 68, 71, 77, 78, 92, and 93 of SEQ ID NO:4.

Thus, in some embodiments, an MSP domain I variant comprises an amino acid sequence from among the sequences shown below:

(SEQ ID NO:61)

(x)Cxxxxx{Z_{I}}CxDxxGxxHxxxxxWx(x)xxxxxCxC{Z_{II}}xCC
(xxxxx);

(SEQ ID NO:62)

(x)C[yfs][fliqt][imqek][prnil][nlrp]{Z_{I}}CxDx[kd]
GxxHxx[ndg][ste]xWx([tn])xxxxxCxC{Z_{II}}[sitra]CC
(xxxxx);

(SEQ ID NO:63)

(x)C[ys][fli][imqe][prn][nlr]{Z_{I}}CxDx[kd]GxxHx
[il][nd][st]xW[qkr](T)[dek]xxxxCxC{Z_{II}}[sit]CC
(xxxxx);

(SEQ ID NO:64)

(S)CxxxPN{Z_{I}}CxDLKGNKHPxxSxWxTxxxxxCxC{Z_{II}}xCC
([ts]Lxx[ti]);

(SEQ ID NO:65)

(S)C[ys][fl][im]PN{Z_{I}}C[mt]DLKGNKHP[il][nd]S[ekr]
W[qkr]T[de][nd]x[de]xCxC{Z_{II}}[si]CC([ts]L[vi][sa]
[ti]);

where “x” is any amino acid. An MSP domain I variant as above will typically have an amino acid sequence that is at least 60%, at least 70%, at least 75%, at least 80%, at least 85%, at least 90%, or at least 95% identical to domain I of a corresponding wild-type MSP protein, exclusive of amino acids of loop regions {Z_{I}} and {Z_{II}}. In certain variations, an MSP domain I variant as above comprises an amino acid sequence as set forth in SEQ ID NO:74, SEQ ID NO:75, SEQ ID NO:76, SEQ ID NO:77, or SEQ ID NO:78.

In other embodiments, an MSP domain II variant comprises an amino acid sequence from among the sequences shown below:

(SEQ ID NO:66)

xxx{Z_{III}}CxxxFxxxxxxxxV{Z_{IV}}xC(xxxxxxx);

(SEQ ID NO:67)

xxx{Z_{III}}C[qrd][rkv][iq]F[knh]x[ek]xx[krt][yi]
[ist]V{Z_{IV}}xC(x[vi]xxWxx);

(SEQ ID NO:68)

xxx{Z_{III}}CxxIFxxExxxxxV{Z_{IV}}xC(xxxxxxx);

(SEQ ID NO:69)

[sa][ti]P{Z_{III}}C[qr][rk]xF[kn][kq]x[det]x[kr]
[yi][is]V{Z_{IV}}[te]C(x[vi]xxxxx);

(SEQ ID NO:70)

xTP{Z_{III}}CQRIFKKExxKYIV{Z_{IV}}TC(xxxxWIx);

(SEQ ID NO:71)

[sa]TP{Z_{III}}CQRIFKKE[de]xKYIV{Z_{IV}}TC(xxx[eq]WI
[il]);

where “x” is any amino acid. An MSP domain II variant as above will typically have an amino acid sequence that is at least 60%, at least 70%, at least 75%, at least 80%, at least 85%, at least 90%, or at least 95% identical to domain II of a corresponding wild-type MSP protein, exclusive of amino acids of loop regions {Z_{III}} and {Z_{IV}} In certain variations, an MSP domain II variant as above comprises an amino acid sequence as set forth in SEQ ID NO:81, SEQ ID NO:82, SEQ ID NO:83, SEQ ID NO:84, SEQ ID NO:85, or SEQ ID NO:86.

In certain cases, suitable amino acids modifications that maintain the three-dimensional structure of a wild-type MSP protein may be selected using protein design or modeling algorithms such as PDA™ technology (see U.S. Pat. Nos. 6,188,965; 6,269,312; and 6,403,312). Algorithms in this class generally use atomic-level or amino acid level scoring functions to evaluate the compatibility of amino acid sequences with the overall tertiary and quaternary structure of a protein. Thus, algorithms of this class can be used to select MSP polypeptide modifications and/or disruptions that do not substantially perturb the ability of variant MSP polypeptides to properly fold and interact with target molecules via one or more of the variable loop regions. These technologies typically use high-resolution structural information of the protein as input. In one embodiment, an experimentally determined structure of the appropriate MSP protein is used as input. In alternative embodiments, an MSA can be used to guide the construction of atomic-level homology models for MSP family members based on the subset of the family whose three-dimensional structures have been determined using crystallographic or related methods.

In some variations, a polypeptide of the invention comprises only one MSP monomer domain variant as described herein. As previously noted, in the full MSP protein, the two domains are held together in part by one disulfide bond between cysteines 37 and 73 (amino acid residue position numbers according to the human MSP protein as set forth in SEQ ID NO:4). In embodiments having a single MSP monomer domain, the unpaired cysteine residue of the unmodified MSP domain is typically substituted with a non-sulfhydryl amino acid residue (i.e., in a domain I variant, the cysteine corresponding to C₃₇ of SEQ ID NO:4 is substituted with a non-sulfhydryl residue; and in a domain II variant, the cysteine corresponding to C₇₃ is substituted with a non-sulfhydryl residue). A suitable non-sulfhydryl residue for substitution at these positions is glycine. Additionally, polypeptides comprising one MSP monomer domain variant may further comprise other polypeptide regions that are heterologous to MSP, such as, for example, an antigen-binding domain and/or a monomer domain derived from a non-MSP protein. Monomer domains derived from non-MSP proteins include, for example, domains derived from the Z-domain of protein A; immunity proteins such as E. coli colicin E7 (ImmE7); cytochrome b₅₆₂; peptide α₂p8; repeat-motif proteins such as ankrin repeats; insect defensin A (IICA₂₉); kunitz domains such as BPTI/APPA; PDZ domains such as Ras-binding protein AF-6; scorpion toxins such as charybdotoxin; plant homeodomain (PHD) finger protein; TEM-1 β-lactamase; 10th fibronectin type III domain (¹⁰Fn3); the extracellular domain of CTLA-4; T-cell receptors; knottins such as Min-23 and cellulose binding domain; neocarzinostatin; CBM-2; tendamistat; lipocalins such as apolipoprotein D, bilin-binding protein, and FABP. (See generally, e.g., Hosse et al., Protein Sci. 15:14-27, 2006.)

In other variations, a polypeptide of the invention is a multimer comprising a plurality of MSP monomer domain variants (e.g., two, three, four, or more monomer domain variants). In these embodiments, each MSP monomer domain variant can be either an MSP domain I variant or an MSP domain II variant as described herein. For example, in more specific embodiments, a multimer having two MSP monomer domains can have either two domain I variants, two domain II variants, or one domain I variant and one domain II variant.

In yet other variations, a polypeptide of the invention is a multimer comprising (a) at least one MSP monomer domain variant and (b) at least one other domain, which may be a monomer domain (whether derived from an MSP or a non-MSP protein), and/or an antigen-binding domain (i.e., a protein binding domain that contains at least one complementarity determining region (CDR) of an antibody and capable of binding to its antigen, which includes antigen-binding fragments of naturally-occurring antibodies and engineered variants thereof, see supra).

Individual monomer domains and/or antibodies of a multimer can be linked via a polypeptide linker, such as described further herein, infra.

In specific embodiments of a polypeptide comprising both an MSP domain I variant and an MSP domain II variant, the domain I and domain II variants are linked via the natural connection between domains I and II of the corresponding wild-type MSP protein, with domain II positioned C-terminal to domain I. Accordingly, in such embodiments, the multimer comprising domains I and II will include amino acids of the wild-type MSP protein corresponding to amino acids 51-55 of human MSP (SEQ ID NO:4), which defines the polypeptide segment between the most C-terminal cysteine of domain I (corresponding to C₅₀ of SEQ ID NO:4) and loop III of domain II (corresponding to amino acid residues 57-63 of SEQ ID NO:4). As domain I variants may optionally include C-terminal amino acid residues corresponding to residues 51-55 of SEQ ID NO:4, multimers comprising this natural linkage between domains I and II essentially comprise the domain II variant positioned C-terminal and directly adjacent to the domain I variant.

Alternatively, a multimer comprising both domain I and domain II may be linked via a polypeptide linker heterologous to the wild-type MSP protein, as discussed further infra.

Furthermore, in certain embodiments, a multimer comprising both domain I and domain II comprises the native cysteine residues at positions corresponding to positions 37 and 73 of SEQ ID NO:4. For example, in specific embodiments, a multimer comprising both a domain I and a domain II variant, each derived from the human MSP protein of SEQ ID NO:4, retains cysteines C₃₇ (domain I) and C₇₃ (domain II) of the human MSP protein. Conservation of these cysteine residues, which further link domain I and domain II in wild-type MSP proteins via disulfide bonding, facilitates conservation of the native MSP three-dimensional structure in multimers comprising both MSP domains, including relative orientations of the variable domain I and II loop regions. Alternatively, as previously noted, these cysteines may be replaced with a non-sulfhydryl-containing residue to provide greater flexibility between domains I and II, thereby allowing each domain to function more independently.

One or more loop regions of an MSP monomer domain variant can be, for example, a randomized polypeptide segment (e.g., a randomized polypeptide segment from among a plurality of randomized segments represented in a library of MSP polypeptide variants). Randomization can be based on full randomization, or optionally, partial randomization (e.g., pseudorandomization or semi-randomization) based on natural distribution of sequence diversity. For example, in certain embodiments, an MSP polypeptide variant is from a library of MSP polypeptide variants in which one or more loop regions ({Z_{I}}, {Z_{II}}, {Z_{III}}, {Z_{IV}}, and/or {Z_{V}}) are substituted with peptides representing all possible amino acid sequences of length N (where N is a positive integer), or a subset of all possible sequences.

Polynucleotides encoding the monomer domains are typically employed to make MSP monomer domain variants and multimer thereof via expression. Nucleic acids that encode MSP polypeptides can be derived from a variety of different sources. Libraries of MSP monomer domain variants, including libraries of multimers comprising one or more MSP monomer domain variants, can be prepared by expressing a plurality of different nucleic acids encoding such MSP variant polypeptides.

MSP monomer domain variants of the present invention can be produced by a number of methods. For example, amino acids may be inserted or exchanged using synthetic oligonucleotides, or by shuffling, or by restriction enzyme based recombination. In certain embodiments, oligonucleotide-based methods are used to substitute heterologous polypeptide segments at one or more of the loop regions ({Z_{I}}, {Z_{II}}, {Z_{III}}, {Z_{IV}}, and/or {Z_{V}}). Such oligonucleotides may be randomized (e.g., to encode a fully randomized, pseudorandomized, or semi-randomized polypeptide segment), and libraries of synthetic oligonucleotides may be generated for incorporation into an MSP gene sequence in a site-directed manner at one or more loop regions. As the essence of oligonucleotide-directed methods is that a synthetic DNA sequence is incorporated into a “full-length” gene sequence, any form of randomization that can be achieved in a synthetic DNA fragment can be replicated in an MSP gene sequence (e.g., a gene sequence encoding an MSP polypeptide comprising one or both of domain I and domain II). A wide range of synthetic strategies are available that allow highly precise and controlled randomization within oligonucleotides.

Regardless of how an oligonucleotide is synthesized, once synthesized, a wide variety of site-directed methods is available for incorporation of the synthetic DNA sequence into a gene sequence encoding an MSP polypeptide. Such methods include, for example, PCR based techniques such as strand overlap extension (SOE) and megaprimer based procedures. (See, e.g., Ho et al., Gene 77:51-59, 1989; Landt et al., Gene 96:125-128, 1990.) Mutagenic plasmid amplification (MPA, marketed in kit form by Stratagene as QuikChange™ system) and related methods may also be used (see, e.g., Zheng et al., Nucleic Acids Research 32:e115, 2004). The QuikChange™ system can also be used with megaprimers, meaning that only one mutagenic primer is required. Synthetic primers can also be incorporated via a number of recombination strategies, including, for example, DNA shuffling techniques. (See, e.g., Ness et al., Nat. Biotechnol. 20:1251-1255, 2002. See also Yuan et al., Microbiol. Mol. Biol. Rev. 69:373-392, 2005.)

Randomizing multiple regions of an MSP gene sequence requires either multiple rounds of mutagenesis or more complex methods. For example, the Assembly of Designed Oligonucleotides method (see Zha et al., Chembiochem. 4:34-39, 2003) and synthetic shuffling (see Ness et al., supra) utilize a shuffling-type approach with synthetic oligonucleotides, and multiple mutagenic primers can be used with, e.g., QuikChange™ system to randomize multiple positions (see, e.g., Jensen and Weilguny, J. Biomol. Tech. 16:336-340, 2005). Another, similar approach is to construct overlapping gene segments by PCR with mutagenic primers and then reconstruct these in an overlap extension reaction. If a small number of gene fragments (e.g., up to 4) are used, then the reconstruction can be performed by strand extension rather than PCR amplification (i.e., without external primers).

Methods of mutagenesis, such as site-directed mutagenesis, can also be used to introduce substitutions, additions, or deletions in regions outside of loop regions {Z_{I}}{Z_{II}}{Z_{III}}{Z_{IV}}, and {Z_{V}}. Such methods can include, e.g., aligning a plurality of naturally occurring MSP monomer domains by aligning conserved amino acids in the plurality of naturally occurring monomer domains; and designing the MSP monomer domain variant by maintaining the conserved amino acids and inserting, deleting, or altering amino acids around the conserved amino acids to generate the MSP monomer domain variant. As previously noted, paired cysteines are typically conserved. Examples of other conserved residues may include those enumerated above. Amino acids at relatively non-conserved positions can be altered by, for example, substituting the amino acid at that position with a different amino acid that occurs at that position in one or more MSP proteins from other species, or with an amino acid that represents a conservative amino acid substitution, as previously discussed. Amino acids can also be substituted with any other amino acid that does not substantially disrupt the three-dimensional structure of the domain variant relative to the wild-type protein, as determined, for example, using algorithms such as previously discussed to evaluate the compatibility of amino acid modifications with the overall tertiary and quaternary structure of the MSP protein.

Accordingly, in another aspect of the present invention, also provided are recombinant nucleic acids encoding any of the MSP monomer domain variants or multimers of the present invention. Using a nucleic acid of the present invention, encoding an MSP domain variant or multimer thereof, a variety of vectors can be made. Any vector containing replicon and control sequences that are derived from a species compatible with the host cell can be used in the practice of the invention. Generally, expression vectors include transcriptional and translational regulatory nucleic acid regions operably linked to the nucleic acid encoding an MSP variant polypeptide. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. The transcriptional and translational regulatory nucleic acid regions will generally be appropriate to the host cell used to express the MSP variant polypeptide. Numerous types of appropriate expression vectors and suitable regulatory sequences are known in the art for a variety of host cells. In general, the transcriptional and translational regulatory sequences may include, e.g., promoter sequences, ribosomal binding sites, transcriptional start and stop sequences, translational start and stop sequences, and enhancer or activator sequences. In typical embodiments, the regulatory sequences include a promoter and transcriptional start and stop sequences. Vectors also typically include a polylinker region containing several restriction sites for insertion of foreign DNA. The construction of suitable vectors containing DNA encoding replication sequences, regulatory sequences, phenotypic selection genes, and the MSP variant polypeptide of interest are prepared using standard recombinant DNA procedures. Isolated plasmids, viral vectors, and DNA fragments are cleaved, tailored, and ligated together in a specific order to generate the desired vectors, as is well-known in the art (see, e.g., Sambrook et al., Molecular Cloning: A Laboratory Manual (Cold Spring Harbor Laboratory Press, New York, N.Y., 2nd ed. 1989)).

In certain embodiments, the expression vector contains a selectable marker gene to allow the selection of transformed host cells. Selection genes are well known in the art and will vary with the host cell used. Suitable selection genes can include, for example, genes coding for ampicillin and/or tetracycline resistance, which enables cells transformed with these vectors to grow in the presence of these antibiotics.

In one aspect of the present invention, a nucleic acid encoding a mutant DNA polymerase is introduced into a cell, either alone or in combination with a vector. By “introduced into” or grammatical equivalents herein is meant that the nucleic acids enter the cells in a manner suitable for subsequent integration, amplification, and/or expression of the nucleic acid. The method of introduction is largely dictated by the targeted cell type. Exemplary methods include CaPO₄ precipitation, liposome fusion, LIPOFECTIN®, electroporation, viral infection, and the like.

Prokaryotes are typically used as host cells for the initial cloning steps of the present invention. They are particularly useful for rapid production of large amounts of DNA, for production of single-stranded DNA templates used for site-directed mutagenesis, for screening many MSP variant polypeptides simultaneously, and for DNA sequencing of the MSP variants generated. Suitable prokaryotic host cells include, for example, E. coli strains K12, W3110, X1776, B, HB101, JM101, NM522, NM538, and NM539, as well as many other species and genera of prokaryotes including bacilli such as Bacillus subtilis, other enterobacteriaceae such as Salmonella typhimurium or Serratia marcesans, and various Pseudomonas species. Prokaryotic host cells or other host cells with rigid cell walls are typically transformed using the calcium chloride method as described in section 1.82 of Sambrook et al., supra. Alternatively, electroporation can be used for transformation of these cells. Prokaryote transformation techniques are set forth in, for example Dower, in Genetic Engineering, Principles and Methods 12:275-296 (Plenum Publishing Corp., 1990); Hanahan et al., Meth. Enzymol, 204:63, 1991. Plasmids typically used for transformation of E. coli include pBR322, pUCI8, pUCI9, pUCI118, pUC119, and Bluescript M13, all of which are described in sections 1.12-1.20 of Sambrook et al., supra. However, many other suitable vectors are available as well.

The MSP monomer domain variants and multimers of the present invention are typically produced by culturing a host cell transformed with an expression vector containing a nucleic acid encoding the MSP variant polypeptide under the appropriate conditions to induce or cause expression of the MSP variant polypeptide. Methods of culturing transformed host cells under conditions suitable for protein expression are well-known in the art (see, e.g., Sambrook et al., supra). Following expression, the MSP variant polypeptide can be harvested and isolated.

In some embodiments, E. coli comprising a plasmid encoding the polypeptides under transcriptional control of a bacterial promoter is used to express the protein. After harvesting the bacteria, they may be lysed by sonication, heat, or homogenization and clarified by centrifugation. The polypeptides may be purified using Ni-NTA agarose elution (if 6×His tagged) or DEAE sepharose elution (if untagged) and refolded by dialysis. Misfolded proteins may be neutralized by capping free sulfhydryls with iodoacetic acid. Q sepharose elution, butyl sepharose flow-through, SP sepharose elution, DEAE sepharose elution, and/or CM sepharose elution may be used to purify the polypeptides. Equivalent anion and/or cation exchange or hydrophobic interaction purification steps may also be employed.

In some embodiments, following manufacture of the monomers or multimers of the invention, the polypeptides are treated in a solution comprising iodoacetic acid to cap free —SH moieties of cysteines that have not formed disulfide bonds. In some embodiments, 0.1-100 mM (e.g., 1-10 mM) iodoacetic acid is included in the solutions.

In certain variations, MSP monomer domains or multimers of the invention are expressed using a reporting display vector or system. Such embodiments are particularly useful for screening monomer domain or multimer libraries to select monomers or multimers having, e.g., particular target-binding characteristics. As described further herein, a variety of reporting display vectors or systems can be used to express nucleic acids encoding MSP monomer domain variants and/or other domains, including multimers thereof, to test for a desired activity, including, for example, phage display, ribosome display, nucleotide-linked display, polysome display, cell surface display, and the like. (See, e.g., International Patent Application Publication Nos. WO 91/17271, WO 91/18980, WO 91/19818 and WO 93/08278; and U.S. Pat. Nos. 6,281,344, 6,194,550, 6,207,446, 6,214,553 and 6,258,558). Such display systems are generally well-known in the art and can be readily used for expression and screening of MSP monomer domain variants and multimers in accordance with the present invention.

As described further herein, MSP monomer domain variants and multimers comprising one or more such variants can be selected to bind to a target molecule. Selection for binding to a target can be based, e.g., on inhibiting or enhancing a specific function of a target protein or an activity. Target protein activity can include, for example, endocytosis or internalization, induction of second messenger system, up-regulation or down-regulation of a gene, binding to an extracellular matrix, release of a molecule(s), or a change in conformation.

In some embodiments, an MSP variant polypeptide as described herein is selected to bind to a tissue- or disease-specific target protein. Tissue-specific proteins are proteins that are expressed exclusively, or at a significantly higher level, in one or several particular tissue(s) compared to other tissues in an animal. Similarly, disease-specific proteins are proteins that are expressed exclusively, or at a significantly higher level, in one or several diseased cells or tissues compared to other non-diseased cells or tissues in an animal. Examples of such diseases include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathycandidiasis-ectodermal dystrophy (APECED), bronchitis, cholecystitis, contact dennatitis, Crohn's disease, atopic dennatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, sclerodenna, Sjogren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a cardiovascular disorder such as congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, complications of cardiac transplantation, arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and GerstmannStraussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; and a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodennas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss. Exemplary disease or conditions include, e.g., MS, SLE, ITP, IDDM, MG, CLL, CD, RA, Factor VIII Hemophilia, transplantation, arteriosclerosis, Sjogren's Syndrome, Kawasaki Disease, anti-phospholipid Ab, AHA, ulcerative colitis, multiple myeloma, Glomerulonephritis, seasonal allergies, and IgA Nephropathy.

Exemplary tissue-specific or disease-specific proteins can be found in, e.g., Tables I and II of U.S. Patent Publication No. 2002/0107215. Exemplary tissues where target proteins may be specifically expressed include, for example, liver, pancreas, adrenal gland, thyroid, salivary gland, pituitary gland, brain, spinal cord, lung, heart, breast, skeletal muscle, bone marrow, thymus, spleen, lymph node, colorectal, stomach, ovarian, small intestine, uterus, placenta, prostate, testis, colon, colon, gastric, bladder, trachea, kidney, or adipose tissue.

Multimers

Multimers and methods for generating multimers are also a feature of the present invention. Multimers of the invention generally comprise at least two domains (“subunits”), each domain being either a monomer domain or an antigen-binding domain, and where at least one domain of the multimer is an MSP monomer domain variant as described herein. Accordingly, a multimer can comprise, e.g., at least two monomer domains (at least one of which is an MSP monomer domain variant), or at least one MSP monomer domain variant and at least one antigen-binding domain. In various embodiments, a multimer can comprise from 2 to about 10 subunits, from 2 to about 8 subunits, or from about 3 to about 10 subunits (e.g., in more particular variations, a multimer can comprise 2, 3, 4, 5, 6, 7, 8, 9, or 10 subunits). In certain embodiments, each domain of a multimer is a monomer domain; for example, in some embodiments, each multimer subunit is an MSP monomer domain variant as described herein. In a specific variation, a multimer comprises or consists of two MSP monomer domain variants, one of which is a domain I variant and the other of which is a domain II variant as described herein, which MSP monomer domain variants may be linked either via the natural connection between MSP domains I and II or via a heterologous polypeptide linker. Typically, the domains of a multimer have been pre-selected for binding to the target molecule of interest.

In certain embodiments, each domain of a multimer specifically binds to one target molecule. In some such embodiments, each domain binds to a different position (i.e., epitope) on a target molecule. Multiple domains that bind to the same target molecule result in an avidity effect yielding improved avidity of the multimer for the target molecule compared to each individual domain. In some embodiments, the multimer has an avidity of at least about 1.5, 2, 3, 4, 5, 10, 20, 50, 100, or 1000 times the avidity of an individual domain alone. Typically, the multimer has a K_{d} of less than about 10⁻¹⁵, 10⁻¹⁴, 10⁻¹³, 10⁻¹², 10⁻¹¹, 10⁻¹⁰, 10⁻⁹, or 10⁻⁸.

In another embodiment, the multimer comprises domains with specificities for different target molecules. For example, multimers of such diverse domains can specifically bind different components of a viral replication system or different serotypes of a virus. In some embodiments, at least one domain binds to a toxin and at least one domain binds to a cell surface molecule, thereby acting as a mechanism to target the toxin. In some embodiments, at least two domains of the multimer bind to different target molecules in a target cell or tissue. Similarly, therapeutic molecules can be targeted to the cell or tissue by binding a therapeutic agent to a domain of the multimer that also contains other domains having cell or tissue binding specificity. In some variations, the different domains bind to different components of a signal transduction pathway, a metabolic pathway, or components of different metabolic pathways that exert the same additive or synergistic physiological or biological effect or effects.

Multimers can comprise a variety of combinations of subunits. For example, in a single multimer, the selected domains can be the same or identical; alternatively, the selected domains can be different or non-identical. Thus, in certain variations in which a multimer comprises two or more monomer domains, the selected monomers can be the same or identical or, alternatively, different or non-identical. In addition, the selected monomer domains can include two or more different MSP monomer domains or, alternatively, one or more MSP monomer domains in addition to one or more non-MSP monomer domains (i.e., monomer domains derived from domain families other than the MSP family of proteins).

In certain variations, multimers that are generated in the practice of the present invention may be any of the following:
(1) A homo-multimer (a multimer of the same MSP monomer domain variant (e.g. D1_{α}-D1_{α}-D1α-D1α, where D1α denotes an MSP monomer domain I variant specific for a particular epitope (designated “α”));
(2) A hetero-multimer of different domains of the MSP protein class (e.g., D1α-D1γ, D1α-D2β or D1α-D2_{β}-D3_{γ}-D4₆, where D1_{α} and D1γ denote MSP monomer domain I variants specific for different epitopes (designated “α” and “γ,” respectively); D2_{β} and D4_{δ} denote MSP monomer domain II variants specific for different epitopes (designated “β” and “δ,” respectively); and where two or more of epitopes “α,” “β”, “γ,” and “δ” are optionally epitopes from the same target molecule);
(3) A hetero-multimer of domains from different classes, such as a hetero-multimer comprising an MSP monomer domain variant and a non-MSP domain, which can be either an antigen-binding domain or a monomer domain variant derived from a non-MSP protein (e.g., D1-A1, D1-D2-A1, D1-X1, or D1-D2-X1-X2, where D1 and D2 are, respectively, domain I and II variants from MSP; A1 is an antibody; and X1 and X2 are two different monomer domains from a non-MSP protein).

Multimer libraries employed in the practice of the present invention may contain MSP homo-multimers, hetero-multimers of different domains of the MSP protein class, or hetero-multimers of domains from different classes (such as a hetero-multimer comprising an MSP monomer domain variant and a non-MSP domain), or combinations thereof. Multimers can include dimers, trimers, tetramers, and even higher order multimers.

As previously indicated, MSP monomer domain variants as described herein are readily employed in a heteromultimer containing an antigen-binding domain (i.e., a multimer that has at least one antigen-binding domain and at least one MSP monomer domain variant). Thus, multimers of the present invention may have at least one antigen-binding domain such as, for example, a minibody, a single-domain antibody, a single chain variable fragment (ScFv), or a Fab fragment; and at least one MSP monomer domain variant such as, for example, an MSP domain I variant, and MSP domain II variant, or both.

Multimer domains need not be selected for target specificity before the domains are linked to form multimers. On the other hand, domains can be selected for the ability to bind to a target molecule before being linked into multimers. Thus, for example, a multimer can comprise two domains that bind to one target molecule and a third domain that binds to a second target molecule, each domain having been selected based on target specificity and then joined to form the multimer.

Typically, multimers of the present invention are a single discrete polypeptide. Multimers of [partial linker]-[subunit]-[partial linker] moieties are an association of multiple polypeptides, each polypeptide corresponding to a [partial linker]-[subunit]-[partial linker] moiety. Multimers of the present invention may have, for example, one or more of the following qualities: multivalent, multispecific, single chain, heat stable, extended serum and/or shelf half-life.

In some cases, multimers will bind to two different targets. In some such embodiments, a multimer will bind to two different targets, each on the surface of a cell or, alternatively, one on a cell-surface and another on a non-cell-surface molecule. In certain variations, a multimer will bind to two different targets, each on a different cell-surface (e.g., surfaces of two different cell types). Multimers with affinity for two different targets (e.g., both a cell surface target and a second target) may provide for increased avidity effects. In some cases, membrane fluidity can be more flexible than protein linkers in optimizing (by self-assembly) the spacing and valency of the interactions.

In some embodiments, the monomers or multimers of the present invention are linked to another polypeptide to form a fusion protein. In certain variations, a fusion partner facilitates the formation of a multimeric structure, such as by the formation of one or more disulfide bonds between different monomeric or multimeric subunits. For example, monomers or multimers of the invention may be fused to the following regions or combinations of regions of an antibody:
(a) at the N-terminus of the V_{H1} and/or V_{L1} domains, optionally just after the leader peptide and before the domain starts (framework region 1);
(b) at the N-terminus of the C_{H1} or C_{L1} domain, replacing the V_{H1} or V_{L1} domain;
(c) at the N-terminus of the heavy chain, optionally after the C_{H1} domain and before the cysteine residues in the hinge (Fc-fusion);
(d) at the N-terminus of the C_{H3} domain;
(e) at the C-terminus of the C_{H3} domain, optionally attached to the last amino acid residue via a short linker;
(f) at the C-terminus of the C_{H2} domain, replacing the C_{H3} domain;
(g) at the C-terminus of the C_{L1} or C_{H1} domain, optionally after the cysteine that forms the interchain disulfide; or
(h) at the C-terminus of the V_{H1} or V_{L1} domain.

In some embodiments, an MSP monomer domain variant or multimer is linked to a molecule (e.g., a protein, nucleic acid, organic small molecule, etc.) useful as a pharmaceutical. Exemplary pharmaceutical proteins include, e.g., cytokines, antibodies, chemokines, growth factors, interleukins, cell-surface proteins, extracellular domains, cell surface receptors, cytotoxins, etc. Exemplary small molecule pharmaceuticals include small molecule toxins or therapeutic agents. Such embodiments can be used, e.g., to target such pharmaceuticals to a particular cell type or tissue by using an MSP monomer domain variant or multimer having specificity for that cell or tissue.

Accordingly, in some embodiments, an MSP variant polypeptide that binds to a tissue- or disease-specific target protein is linked to the pharmaceutical protein or small molecule such that the resulting complex or fusion is targeted to a specific tissue or disease-related cell(s) where the target protein is expressed. MSP monomer domain variants and multimers as described herein for use in such complexes or fusions can be initially selected for binding to the target protein and may be subsequently selected by negative selection against other cells or tissue (e.g., to avoid targeting bone marrow or other tissues that set the lower limit of drug toxicity) where it is desired that binding be reduced or eliminated in other non-target cells or tissues. By keeping the pharmaceutical away from sensitive tissues, the therapeutic window is increased so that a higher dose may be administered safely. In another alternative, in vivo panning can be performed in animals by injecting a library of MSP monomer domain variants or multimers into an animal and then isolating the monomers or multimers that bind to a particular tissue or cell of interest.

The fusion proteins described above may also include a linker peptide between the pharmaceutical protein and the MSP monomer domain or multimer. A peptide linker sequence may be employed to separate, for example, the polypeptide components by a distance sufficient to ensure that each polypeptide folds into its secondary and tertiary structures. Fusion proteins may generally be prepared using standard techniques, including chemical conjugation. Fusion proteins can also be expressed as recombinant proteins in an expression system by standard techniques. Suitable linkers are further described herein, infra.

Linkers

As previously noted, an MSP monomer domain variant may be joined to another domain—which may be, e.g., another MSP monomer domain variant, a non-MSP monomer domain, or an antigen-binding domain—to form a single chain multimer. In certain variations comprising both an MSP domain I variant and an MSP domain II variant, the domains are linked via the natural connection between domains I and II of the corresponding wild-type MSP protein. Alternatively, a linker heterologous to the MSP protein may be used. For example, a linker can be positioned between each separate discrete monomer domain in a multimer. Typically, in certain multimer embodiments comprising an antigen-binding domain, the antigen-binding domain is also linked (e.g., to another antigen-binding domain or to a monomer domain) via a linker moiety. Linker moieties that can be readily employed to link antigen-binding domains are the same as those described herein for multimers of monomer domains. Exemplary linker moieties suitable for use in accordance with the present invention are described herein.

Joining MSP monomer domain variants or other domains via a linker can be accomplished using a variety of techniques known in the art. For example, combinatorial assembly of polynucleotides encoding selected monomer domains can be achieved by restriction digestion and re-ligation, by PCR-based, self-priming overlap reactions, or other recombinant methods. The linker can be attached to a monomer domain before the domain is identified for its ability to bind to a target or after the domain has been selected for the ability to bind to a target.

The linker can be naturally-occurring, synthetic, or a combination of both. For example, a synthetic linker can be a randomized linker, e.g., both in sequence and size. In one aspect, the randomized linker can comprise a fully randomized sequence, or optionally, the randomized linker can be based on natural linker sequences. The linker can comprise, for example, a non-polypeptide moiety (e.g., a polynucleotide), a polypeptide, or the like.

A linker can be rigid, or alternatively, flexible, or a combination of both. Linker flexibility can be a function of the composition of both the linker and the subunits that the linker interacts with. The linker joins two selected domains (e.g., two selected monomer domains) and maintains the domains as separate and discrete. The linker can allow the separate, discrete domains to cooperate yet maintain separate properties such as multiple separate binding sites for the same target in a multimer or, for example, multiple separate binding sites for different targets in a multimer. In some cases, a disulfide bridge exists between two linked monomer domains or between a linker and a monomer domain. In some embodiments, the monomer domains and/or linkers comprise metal-binding centers.

Choosing a suitable linker for a specific case where two or more domains are to be connected may depend on a variety of parameters including, e.g., the nature of the domains, the structure and nature of the target to which the polypeptide multimer should bind, and/or the stability of the peptide linker towards proteolysis and oxidation.

The choice of linker can be optimized once the desired domains have been identified. Generally, libraries of multimers having a composition that is fixed with regard to domain composition, but variable in linker composition and length, can be readily prepared and screened as described herein.

Particularly suitable linker polypeptides predominantly include amino acid residues selected from Glycine (Gly), Serine (Ser), Alanine (Ala), and Threonine (Thr). For example, the peptide linker may contain at least 75% (calculated on the basis of the total number of residues present in the peptide linker), such as at least 80%, at least 85%, or at least 90% of amino acid residues selected from Gly, Ser, Ala and Thr. The peptide linker may also consist of Gly, Ser, Ala and/or Thr residues only. The linker polypeptide should have a length that is adequate to link two monomer domains in such a way that they assume the correct conformation relative to one another so that they retain the desired activity, such as binding to a target molecule as well as other activities that may be associated with such target binding (e.g., agonistic or antagonistic activity for a given receptor).

A suitable length for this purpose is, e.g., a length of at least one and typically fewer than about 50 amino acid residues, such as 2-25 amino acid residues, 5-20 amino acid residues, 5-15 amino acid residues, 8-12 amino acid residues or 11 residues. Other suitable polypeptide linker sizes may include, e.g., from about 2 to about 15 amino acids, from about 3 to about 15, from about 4 to about 12, about 10, about 8, or about 6 amino acids. The amino acid residues selected for inclusion in the linker polypeptide should exhibit properties that do not interfere significantly with the activity or function of the polypeptide multimer. Thus, the peptide linker should, on the whole, not exhibit a charge that would be inconsistent with the activity or function of the multimer, or interfere with internal folding, or form bonds or other interactions with amino acid residues in one or more of the domains that would seriously impede the binding of the multimer to the target in question.

In another embodiment of the invention, the peptide linker is selected from a library where the amino acid residues in the peptide linker are randomized for a specific set of subunits (e.g., a specific set of MSP monomer domain variants, such as a specific MSP domain I variant in combination with a specific MSP domain II variant) in a particular polypeptide multimer. A flexible linker could be used to find suitable combinations of domains, which is then optimized using this random library of variable linkers to obtain linkers with optimal length and geometry. The optimal linkers may contain the minimal number of amino acid residues of the right type that participate in the binding to the target and restrict the movement of the domains relative to each other in the polypeptide multimer when not bound to the target.

The use of naturally occurring as well as artificial peptide linkers to connect polypeptides into novel linked fusion polypeptides is well-known in the art. (See, e.g., Hallewell et al., J. Biol. Chem. 264, 5260-5268, 1989; Alfthan et al., Protein Eng. 8, 725-731, 1995; Robinson and Sauer, Biochemistry 35, 109-116, 1996; Khandekar et al., J. Biol. Chem. 272, 32190-32197, 1997; Fares et al., Endocrinology 139, 2459-2464, 1998; Smallshaw et al., Protein Eng. 12, 623-630, 1999; U.S. Pat. No. 5,856,456.)

One example where the use of peptide linkers is widespread is for production of single-chain antibodies where the variable regions of a light chain (V_{L}) and a heavy chain (V_{H}) are joined through an artificial linker, and a large number of publications exist within this particular field. A widely used peptide linker is a 15 mer consisting of three repeats of a Gly-Gly-Gly-Gly-Ser amino acid sequence ((Gly₄Ser)₃) (SEQ ID NO:50). Other linkers have been used, and phage display technology, as well as selective infective phage technology, has been used to diversify and select appropriate linker sequences (Tang et al., J. Biol. Chem. 271, 15682-15686, 1996; Hennecke et al., Protein Eng. 11, 405-410, 1998). Peptide linkers have been used to connect individual chains in hetero- and homo-dimeric proteins such as the T-cell receptor, the lambda Cro repressor, the P22 phage Arc repressor, IL-12, TSH, FSH, IL-5, and interferon-γ. Peptide linkers have also been used to create fusion polypeptides. Various linkers have been used, and, in the case of the Arc repressor, phage display has been used to optimize the linker length and composition for increased stability of the single-chain protein (see Robinson and Sauer, Proc. Natl. Acad. Sci. USA 95, 5929-5934, 1998).

Another type of linker is an intein, i.e., a peptide stretch that is expressed with the single-chain polypeptide, but removed post-translationally by protein splicing. The use of inteins is reviewed by F. S. Gimble in Chemistry and Biology, 5:251-256, 1998.

Still another way of obtaining a suitable linker is by optimizing a simple linker (e.g. (Gly₄Ser)ₙ) through random mutagenesis.

As discussed above, it is generally preferred that the peptide linker possess at least some flexibility. Accordingly, in some variations, the peptide linker contains 1-25 glycine residues, 5-20 glycine residues, 5-15 glycine residues, or 8-12 glycine residues. Particularly suitable peptide linkers typically contain at least 50% glycine residues, such as at least 75% glycine residues. In some embodiments, a peptide linker comprises glycine residues only.

In certain variations, the peptide linker comprises other residues in addition to the glycine. Preferred residues in addition to glycine include Ser, Ala and Thr, particularly Ser. One example of a specific peptide linker includes a peptide linker having the amino acid sequence Glyₓ-Xaa-Gly_{y}-Xaa-Glyₓ (SEQ ID NO:51), wherein each Xaa is independently selected from Alanine (Ala), Valine (Val), Leucine (Leu), Isoleucine (Ile), Methionine (Met), Phenylalanine (Phe), Tryptophan (Trp), Proline (Pro), Glycine (Gly), Serine (Ser), Threonine (Thr), Cysteine (Cys), Tyrosine (Tyr), Asparagine (Asn), Glutamine (Gln), Lysine (Lys), Arginine (Arg), Histidine (His), Aspartate (Asp), and Glutamate (Glu), and wherein x, y, and z are each integers in the range from 1-5. In some embodiments, each Xaa is independently selected from the group consisting of Ser, Ala and Thr. In a specific variation, each of x, y, and z is equal to 3 (thereby yielding a peptide linker having the amino acid sequence Gly-Gly-Gly-Xaa-Gly-Gly-Gly-Xaa-Gly-Gly-Gly (SEQ ID NO:52), wherein each Xaa is selected as above).

In some cases, it may be desirable or necessary to provide some rigidity into the peptide linker. This may be accomplished by including proline residues in the amino acid sequence of the peptide linker. Thus, in another embodiment, a peptide linker comprises at least one proline residue in the amino acid sequence of the peptide linker. For example, a peptide linker can have an amino acid sequence wherein at least 25% (e.g., at least 50% or at least 75%) of the amino acid residues are proline residues. In one particular embodiment of the invention, the peptide linker comprises proline residues only.

In some embodiments, a peptide linker is modified in such a way that an amino acid residue comprising an attachment group for a non-polypeptide moiety is introduced. Examples of such amino acid residues may be a cysteine or a lysine residue (to which the non-polypeptide moiety is then subsequently attached). Another alternative is to include an amino acid sequence having an in vivo N-glycosylation site (thereby attaching a sugar moiety (in vivo) to the peptide linker). An additional option is to genetically incorporate non-natural amino acids using evolved tRNAs and tRNA synthetases (see, e.g., U.S. Patent Application Publication 2003/0082575) into the monomer domains or linkers. For example, insertion of keto-tyrosine allows for site-specific coupling to expressed monomer domains or multimers.

In certain variations, a peptide linker comprises at least one cysteine residue, such as one cysteine residue. For example, in some embodiments, a peptide linker comprises at least one cysteine residue and amino acid residues selected from the group consisting of Gly, Ser, Ala, and Thr. In some such embodiments, a peptide linker comprises glycine residues and cysteine residues, such as glycine residues and cysteine residues only. Typically, only one cysteine residue will be included per peptide linker. One example of a specific peptide linker comprising a cysteine residue includes a peptide linker having the amino acid sequence Glyₙ-Cys-Glyₘ (SEQ ID NO:53), wherein n and m are each integers from 1-12, e.g., from 3-9, from 4-8, or from 4-7. In a specific variation, such a peptide linker has the amino acid sequence GGGGG-C-GGGGG (SEQ ID NO:54).

Introduction of an amino acid residue comprising an attachment group for a non-polypeptide moiety may also be used for the more rigid proline-containing linkers. Accordingly, a peptide linker may comprise proline and cysteine residues, such as proline and cysteine residues only. An example of a specific proline-containing peptide linker comprising a cysteine residue includes a peptide linker having the amino acid sequence Proₙ-Cys-Prom (SEQ ID NO:55), wherein n and m are each integers from 1-12, typically from 3-9, such as from 4-8 or from 4-7. In a specific variation, such a peptide linker has the amino acid sequence PPPPP-C-PPPPP (SEQ ID NO:56).

In some embodiments, the purpose of introducing an amino acid residue comprising an attachment group for a non-polypeptide moiety (e.g., a cysteine residue as discussed above) is to subsequently attach a non-polypeptide moiety to said residue. For example, non-polypeptide moieties can improve the serum half-life of the polypeptide multimer. Thus, in certain embodiments, a cysteine residue of a polypeptide linker is covalently attached to a non-polypeptide moiety. Preferred examples of non-polypeptide moieties include polymer molecules, such as, e.g., PEG or mPEG.

As previously noted, another possibility of introducing a site-specific attachment group for a non-polypeptide moiety in the peptide linker is to introduce an in vivo N-glycosylation site, such as one in vivo N-glycosylation site, in the peptide linker. For example, an in vivo N-glycosylation site may be introduced in a peptide linker comprising amino acid residues selected from the group consisting of Gly, Ser, Ala and Thr. It will be understood that in order to ensure that a sugar moiety is in fact attached to the in vivo N-glycosylation site, the nucleotide sequence encoding the polypeptide multimer must be inserted in a glycosylating, eukaryotic expression host. A specific example of a peptide linker comprising an in vivo N-glycosylation site is a peptide linker having the amino acid sequence Glyₙ-Asn-Xaa-Ser/Thr-Glyₘ (SEQ ID NO:57), preferably Glyₙ-Asn-Xaa-Thr-Glyₘ (SEQ ID NO:58), wherein Xaa is any amino acid residue except proline, and wherein n and m are each integers in the range from 1-8, preferably in the range from 2-5.

Often, the amino acid sequences of all peptide linkers present in a multimer will be identical. Nevertheless, in certain embodiments, the amino acid sequences of two or more peptide linkers present in a multimer (e.g., peptide linkers multimer having three, four, or more monomer domains) may be different. For example, in certain variations, it will be desirable or necessary to attach only a few, typically only one, non-polypeptide moieties/moiety (such as mPEG or a sugar moiety) to a multimer in order to achieve the desired effect, such as prolonged serum-half life. In the particular case of a tri-mer containing two peptide linkers, only one peptide linker is typically required to be modified (e.g., by introduction of a cysteine residue), whereas modification of the other peptide linker will typically not be necessary. In such a case, the peptide linkers of the polypeptide multimer are different.

Suitable linkers employed in the practice of the present invention can also include an obligate heterodimer of partial linker moieties. The term “obligate heterodimer” (also referred to as “affinity peptides”) refers herein to a dimer of two partial linker moieties that differ from each other in composition, and which associate with each other in a non-covalent, specific manner to join two domains together. The specific association is such that the two partial linkers associate substantially with each other as compared to associating with other partial linkers. Thus, in contrast to multimers of the present invention that are expressed as a single polypeptide, multimers of domains that are linked together via heterodimers are assembled from discrete [partial linker]-[subunit]-[partial linker] units (e.g., [partial linker]-[monomer domain]-[partial linker] units and/or [partial linker]-[antigen-binding domain]-[partial linker] units). Assembly of the heterodimers can be achieved by, for example, mixing. Thus, if the partial linkers are polypeptide segments, each [partial linker]-[subunit]-[partial linker] unit may be expressed as a discrete polypeptide prior to multimer assembly. A disulfide bond can be added to covalently lock the peptides together following the correct non-covalent pairing. Partial linker moieties that are appropriate for forming obligate heterodimers include, for example, polynucleotides, polypeptides, and the like. For example, when the partial linker is a polypeptide, binding domains are produced individually along with their unique linking peptide (i.e., a partial linker) and later combined to form multimers. (See, e.g., Madden et al., Peptide linkers: Unique self-associative high-affinity peptide linkers, Thirteenth American Peptide Symposium, Edmonton, Canada, 1993 (abstract).) The spatial order of the binding domains in the multimer is thus mandated by the heterodimeric binding specificity of each partial linker. Partial linkers can contain terminal amino acid sequences that specifically bind to a defined heterologous amino acid sequence. An example of such an amino acid sequence is the Hydra neuropeptide head activator as described in Bodenmuller et al (EMBO J. 5(8):1825-1829, 1986). (See, e.g., U.S. Pat. No. 5,491,074 and WO 94/28173.) These partial linkers allow the multimer to be produced first as [subunit]-[partial linker] units or [partial linker]-[subunit]-[partial linker] units that are then mixed together and allowed to assemble into the ideal order based on the binding specificities of each partial linker. Alternatively, domains linked to partial linkers can be contacted to a surface, such as a cell, in which multiple domains can associate to form higher avidity complexes via partial linkers. In some cases, the association will form via random Brownian motion.

In certain embodiments, when a partial linker comprises a DNA binding motif, each domain has an upstream and a downstream partial linker (i.e., Lp-domain-Lp, where “Lp” is a representation of a partial linker) that contains a DNA binding protein with exclusively unique DNA binding specificity. These domains can be produced individually and then assembled into a specific multimer by the mixing of the subunits with DNA fragments containing the proper nucleotide sequences (i.e., the specific recognition sites for the DNA binding proteins of the partial linkers of the two desired domains) so as to join the domains in the desired order. Additionally, the same domains may be assembled into many different multimers by the addition of DNA sequences containing various combinations of DNA binding protein recognition sites. Further randomization of the combinations of DNA binding protein recognition sites in the DNA fragments can allow the assembly of libraries of multimers. The DNA can be synthesized with backbone analogs to prevent degradation in vivo.

Methods for generating a multimer from individual domains can include joining selected domains with a linker to generate the multimer. For example, a multimer can be generating by joining at least two monomer domains, or at least one monomer domain and at least one antigen-binding domain, and the linker. The multimer is then screened for an improved avidity or affinity or altered specificity for a target or mixture of targets as compared, e.g., to the selected domains individually. This process of linking selected polypeptide subunits and screening for improved avidity, affinity, or altered specificity for a target or target mixture can be repeated in an iterative fashion. For example, in some methods, at least two multimers selected as above are joined with a linker to generate a new multimer comprising the selected multimers, each selected multimer comprising at least two domains (e.g., at least two monomer domains or at least one monomer domain and at least one antigen-binding domain). The new multimer can then be screened for an improved avidity or affinity or altered specificity for the target or mixture of targets as compared to the selected domains individually, or as compared to the selected multimers from which the new multimer was generated.

### III. Polypeptide and Nucleic Acid Libraries

The present invention also provides libraries of polypeptides comprising MSP monomer domain variants, including polypeptides comprising multimers as described herein, as well as libraries of nucleic acids that encode such MSP variant polypeptides. The libraries can include, e.g., a pool of about 10, 100, 250, 500, 1000, or 10,000 or more different polypeptides comprising an MSP monomer domain variant, or of pool of about 10, 100, 250, 500, 1000, or 10,000 or more different nucleic acids encoding different polypeptides comprising an MSP monomer domain variant. Methods for producing a library as described herein, one or more cell(s) comprising one or more members of the library, and one or more displays comprising one or more members of the library are also included in the present invention.

In certain embodiments, a polypeptide library is provided comprising a pool of different polypeptides, where each polypeptide of the polypeptide pool is a polypeptide comprising at least one MSP monomer domain variant as described herein (i.e., at least one MSP domain I variant and/or MSP domain II variant), and where at least one of the loop regions present in each polypeptide (i.e., one or more of the {Z_{I}}, {Z_{II}}, {Z_{III}}, {Z_{IV}}, and {Z_{V}} loop regions) is different among different polypeptides of the polypeptide pool. In some variations of the polypeptide library, each polypeptide of the polypeptide pool is a monomer (i.e., a monomer comprising only one MSP domain variant). Alternatively, each polypeptide of the polypeptide pool can be a multimer such as, for example, a polypeptide comprising at least two MSP monomer domain variants.

Accordingly, in some embodiments, MSP polypeptide variants are generated by recombining two or more different MSP monomer domain sequences. In such variations, each of the MSP monomer domain variants can be a domain I variant or a domain II variant. Alternatively, library polypeptides of the invention can include both a domain I variant and a domain II variant. Such multimers can further include one or more domains heterologous to MSP (“non-MSP” domains) such as an antigen-binding domain or a monomer domain derived from a non-MSP protein. In the case of polypeptide libraries comprising different multimers, individual domains (monomer domains and/or antigen-binding domains) of a multimer can be linked via a polypeptide linker, such as described supra. As previously discussed, in specific embodiments of a polypeptide comprising both an MSP domain I variant and an MSP domain II variant, the domain I and domain II variants may be linked via the natural connection between domains I and II of the corresponding wild-type MSP protein, with domain II positioned C-terminal to domain I. Alternatively, a linker heterologous to the MSP protein may be used.

Methods for generating libraries are generally well-known and are readily adaptable for use in preparing libraries of nucleic acids that encode variant MSP polypeptides, including multimers, as described herein. For example, in certain embodiments, for generation of MSP monomer domain variants having different loop region sequences (i.e., one or more of the {Z_{I}}, {Z_{II}}, {Z_{III}}, {Z_{IV}}, and {Z_{V}} loop regions), random, pseudo-random, or semi-random synthetic oligonucleotides are prepared. These oligonucleotide sequences are incorporated into a plurality of nucleic acids encoding an MSP polypeptide, at sites corresponding to one or more loop regions, using any of a wide variety of available site-directed methods, such as methods discussed herein. As noted previously, such methods include, for example, PCR based techniques such as strand overlap extension (SOE) and megaprimer based procedures, mutagenic plasmid amplification (MPA), and recombination techniques such as, for example, DNA shuffling techniques. The different MSP nucleic acids generated by such procedures are incorporated into copies of an expression vector (e.g., a phage display vector) to form an expression library. The expression library is introduced into a suitable host strain, such as an E. coli strain, and clones are selected. The number of individual clones is typically sufficient to achieve reasonable coverage of the possible permutations of the starting material. The clones are combined and grown in mass culture, or in pools, for isolation of the resident vectors and their inserts. This process allows large quantities of the expression library to be obtained in preparation for subsequent procedures described herein. The details of manipulating and cloning oligonucleotides are known in the art, as well as the details of library construction, manipulation, and maintenance. (See, e.g., Sambrook et al., Molecular Cloning: A Laboratory Manual (3rd ed., Cold Spring Harbor Laboratory Press, New York 2001); Ausubel et al., Current Protocols in Molecular Biology (4th ed. John Wiley and Sons, New York 1999).

With regard to sequence diversity for any varied loop region sequence (e.g., one or more of loops I, II, III, IV, or V), such diversity at each position can represent all possible amino acids or, alternatively, a subset of all amino acids. For example, in some variations, the sequence diversity is four-fold at each position (e.g., incorporating one of the amino acids alanine (A), aspartic acid (D), serine (S), and tyrosine (Y) at each position).

In addition, amino acids at each position may be represented in equal or in unequal proportions. In one specific embodiment, for example, sequence the diversity is four fold at each position, incorporating the amino acids alanine, aspartic acid, serine, and tyrosine in equal proportions. In an alternative variation, sequence diversity is four fold at each position incorporating the amino acids alanine, aspartic acid, serine, and tyrosine in the following proportions: 6% alanine, 14% aspartic acid, 24% serine, and 56% tyrosine.

As previously indicated, in certain variations, libraries are constructed based on a substantially full length MSP protein (comprising both domains I and II of MSP). In some such embodiments, sequence diversity is introduced into either or both of loops I and II of domain I and into either or both of domains III and IV of domain II; according to the NMR structure of MSP, this approach would create variable binding surfaces at opposite ends of the full length molecule. In other variations, sequence diversity is introduced into loop I, loop II, and loop IV, but the wild-type sequence is retained at loop III, and additional sequence diversity is introduced into the loop between β2 and β3 (“loop V”); according to the NMR structure of MSP, this approach would create a large variable binding surface. For example, in a specific embodiment of a library based on the a full length MSP protein, the sequence diversity is four-fold at each position, incorporating the amino acids alanine, aspartic acid, serine and tyrosine in equal proportions. In an alternative embodiment, sequence diversity is four-fold at each position incorporating the amino acids alanine, aspartic acid, serine and tyrosine in the following proportions: 6% alanine, 14% aspartic acid, 24% serine and 56% tyrosine. According to the NMR structure of MSP94, this approach would create a large variable binding surface.

Libraries of the invention can be screened to identity an MSP polypeptide variant having a desired property, such as, e.g., binding of to a desired target or mixture of targets, or otherwise exposed to selective conditions. For example, members of a library can be displayed and prescreened for binding to a target or a mixture of targets or incubated in serum to remove those clones that are sensitive to serum proteases. MSP polypeptide variants identified as having binding affinity for the target or mixture of targets can modified (e.g., mutagenized or linked to other polypeptide subunits) and the new MSP polypeptide variant can be screened again for binding to the target or mixture of targets with an improved affinity, avidity, or other desired property. For example, selected monomer domains can be combined or joined to form multimers, which can then be screened for an improved affinity or avidity or altered specificity for the target or the mixture of targets. Altered specificity can mean that the specificity is broadened (e.g., binding of multiple related viruses), or optionally, altered specificity can mean that the specificity is narrowed (e.g., binding within a specific region of a target). Those of skill in the art will recognize that there are a number of methods available to calculate avidity. (See, e.g., Mammen et al., Angew Chem Int. Ed. 37:2754-2794, 1998; Muller et al., Anal Biochem. 261:149-158, 1998.)

### IV. Methods of Identifying MSP Monomer Domain Variants and/or Multimers with a Desired Binding Characteristic

The present invention further provides methods of identifying MSP monomer domain variants, as well as multimers comprising such MSP domain variants, that bind to a selected target or mixture of targets. In some embodiments, MSP monomer domain variants, and optionally other individual domains (e.g., non-MSP monomer domains and/or antigen-binding domains), are identified or selected for a desired property (e.g., binding affinity) and then the domains are incorporated as subunits into multimers. For those embodiments, any method resulting in selection of domains with a desired property (e.g., a specific binding property) can be used. For example, the methods can comprise providing a plurality of different nucleic acids, each nucleic acid encoding a monomer domain; translating the plurality of different nucleic acids, thereby providing a plurality of different monomer domains; screening the plurality of different monomer domains for binding of the desired target or a mixture of targets; and identifying members of the plurality of different monomer domains that bind the desired target or mixture of targets.

In certain variations, the MSP monomer domain variants and/or other domains are selected from one or more libraries of domains. For example, in some embodiments, an MSP monomer domain variant is selected from a polypeptide library that comprises a pool of different polypeptides, wherein each polypeptide of the polypeptide pool is a polypeptide comprising an MSP monomer domain variant as described herein, and wherein at least one of loop regions {Z_{I}}, {Z_{II}}, {Z_{III}}, {Z_{IV}}, and {Z_{V}} is different among different polypeptides of the polypeptide pool. The different polypeptides of the polypeptide pool can comprise an MSP domain I variant, an MSP domain II variant, or both an MSP domain I variant and an MSP domain II variant. In some such embodiments, each polypeptide of the polypeptide pool comprises only one MSP monomer domain variant (i.e., either one MSP domain I variant or one MSP domain II variant). Alternatively, each polypeptide of the polypeptide pool can comprise at least two MSP monomer domain variants; for example, in certain embodiments, each polypeptide comprises one MSP domain I variant and one MSP domain II variant, which may be linked via a polypeptide linker heterologous to the wild-type MSP protein or via the natural connection between domains I and II of the wild-type MSP protein, as described previously.

Selection of an MSP monomer domain variant or other domain from a library of domains can be accomplished by a variety of procedures. For example, one method of identifying an MSP monomer domain variant that has a desired property involves translating a plurality of nucleic acids, where each nucleic acid encodes an MSP monomer domain variant, screening the polypeptides encoded by the plurality of nucleic acids, and identifying those monomer domain variants that, e.g., bind to a desired target or mixture of targets, thereby selecting an MSP monomer domain variant. The MSP monomer domain variants expressed by each of the nucleic acids can be tested for their ability to bind to the target or mixture of targets by methods known in the art (e.g., panning, affinity chromatography, FACS analysis).

As mentioned above, selection of MSP monomer domain variants and/or other domains can be based on binding to a target such as a target protein or other target molecule (e.g., lipid, carbohydrate, nucleic acid, and the like). In typical embodiments, the target is a known (i.e., predetermined) target molecule; in alternative variations, the desired target can be an unknown target molecule. Other molecules can optionally be included in the methods along with the target, e.g., ions such as Ca⁺². Other selections of MSP monomer domain variants and/or other domains can be based, e.g., on inhibiting or enhancing a specific function of a target molecule or an activity. Target molecule activity can include, for example, endocytosis or internalization, induction of second messenger system, up-regulation or down-regulation of a gene, binding to an extracellular matrix, release of a molecule, or a change in conformation; in such variations, the target molecule does not need to be known. The selection can also include using high-throughput assays.

When a MSP monomer domain variant and/or other domain is selected based on its ability to bind to a target, the selection basis can include selection based on a slow dissociation rate, which is usually predictive of high affinity. The valency of the target can also be varied to control the average binding affinity of selected MSP monomer domains and/or other domains. The target can be bound to a surface or substrate at varying densities, such as by including a competitor compound, by dilution, or by other methods known to those in the art. High density (valency) of a predetermined target can be used to enrich for domains that have relatively low affinity, whereas a low density (valency) can preferentially enrich for higher affinity domains.

A variety of reporting display vectors or systems can be used to express nucleic acids encoding MSP monomer domain variants and/or other domains, including multimers thereof, to test for a desired activity. For example, a phage display system is a system in which polypeptides are expressed as fusion proteins on the phage surface (Pharmacia, Milwaukee Wis.). Phage display can involve the presentation of a polypeptide sequence encoding MSP monomer domain variants and/or other domains, including multimers, on the surface of a filamentous bacteriophage, typically as a fusion with a bacteriophage coat protein. Generally in these methods, each phage particle or cell serves as an individual library member displaying a single species of displayed polypeptide in addition to the natural phage or cell protein sequences. Nucleic acid molecules, encoding different polypeptides for display, are cloned into the phage DNA at a site that results in the transcription of a fusion protein from each phage containing a nucleic acid molecule, where a portion of the fusion protein is encoded by the nucleic acid molecule. The phage containing a nucleic acid molecule undergoes replication and transcription in the cell. The leader sequence of the fusion protein directs the transport of the fusion protein to the tip of the phage particle. Thus, the fusion protein that is partially encoded by the nucleic acid molecule is displayed on the phage particle for detection and selection by the methods described herein. For example, the phage library can be incubated with a predetermined (desired) target, so that phage particles presenting a fusion protein sequence that binds to the target can be differentially partitioned from those that do not present polypeptide sequences that bind to the predetermined target. For example, the separation can be provided by immobilizing the predetermined target. The phage particles (i.e., library members) that are bound to the immobilized ligand are then recovered and replicated to amplify the selected phage subpopulation for a subsequent round of affinity enrichment and phage replication. After several rounds of affinity enrichment and phage replication, the phage library members that are thus selected are isolated and the nucleotide sequence encoding the displayed polypeptide sequence is determined, thereby identifying the sequence(s) of polypeptides that bind to the predetermined ligand. Such methods are further described in, for example International Patent Application Publication Nos. WO 91/17271, WO 91/18980, WO 91/19818 and WO 93/08278.

Examples of other display systems include ribosome displays, a nucleotide-linked display (see, e.g., U.S. Pat. Nos. 6,281,344; 6,194,550; 6,207,446; 6,214,553; and 6,258,558), polysome display, cell surface displays, and the like. The cell surface displays include a variety of cells, including, e.g., E. coli, yeast, and/or mammalian cells. When a cell is used as a display, nucleic acids encoding polypeptides for display are introduced into the cell and translated. Optionally, nucleic acids encoding the polypeptides can be introduced, e.g., by injection, into the cell.

Those of skill in the art will recognize that the steps of generating variation and screening for a desired property can be repeated (i.e., performed recursively) to optimize results. For example, in a phage display library or other like format, a first screening of a library can be performed at relatively lower stringency, thereby selecting as many particles associated with a target molecule as possible. The selected particles can then be isolated and the polynucleotides encoding the monomer or multimer can be isolated from the particles. Additional variations can then be generated from these sequences and subsequently screened at higher affinity.

MSP monomer domain variants, non-MSP domains, and multimers thereof may be selected to bind any type of target molecule, including protein targets. Suitable target molecules include, for example, various secreted factors such as, e.g., cytokines and chemokines, as well as cell-surface receptors for such secreted factors. Other suitable target molecules include, for example, 4-transmembrance receptors and 7-transmembrance receptors (e.g., G-protein-coupled receptors). Exemplary targets include, but are not limited to, e.g., CD2, CD3, CD8, CD10, CD19, CD20, CD21, CD22, CD23, CD24, CD25, CD28, CD30, CD33, CD37, CD38, CD40, CD45Ro, CD48, CD52, CD55, CD59, CD70, CD74, CD80, CD86, CD138, CD147, Fas (CD95), CTLA-4 (CD152), CD40L (CD154), HLA-DR, CEA, CSAp, CA-125, TAG-72, IL-6, Alpha3, cMet, ICOS, IgE, IL-1-R11, BLys, APRIL, BAFF, Her2, Her3, Her4, IGF-1R, MUC, MUC2, MUC3, MUC4, TNFR1, TNFR2, NGFR, TRAIL-R, DR3, DR4, DR5, DR6, VEGF, TPO-R, TNF-α, LFA-1, TACI, IL-1β, OX40, IL-17A, IL-17F, IL-21, IL-22, IL-23, IL-31, PDGF-Rβ, PDGFRα, PDGF-A, PDGF-B, PDGF-C, PDGF-D, EGF-R, IL-17RA, IL-17RC, IL-21R, IL-22R, IL-23R, and Notch-3. When the target is a receptor for a ligand, the monomer domains may act as antagonists or agonists of the receptor.

When multimers capable of binding relatively large targets are desired, they can be generated by a “walking” selection method. This method is carried out by providing a first library of polypeptide domains (e.g., a library of MSP monomer domain variants) and screening the first library for affinity to a first target molecule. Once at least one domain that binds to the target is identified, that particular domain is covalently linked to each member of a second library or each remaining member of the first library of polypeptide domains to generate a new library. The new library members each comprise one common domain and at least one domain that that is different. The new library of multimers is then screened for multimers that bind to the target with an increased affinity or avidity, and a multimer that binds to the target with an increased affinity or avidity can be identified. This method can be performed recursively to add more domains thereby resulting in a multimer comprising 2, 3, 4, 5, 6, 7, 8 or more domains linked together. The “walking” selection method provides a way to assemble a multimer that is composed of monomers that can act additively or even synergistically with each other given the restraints of linker length. This walking technique is very useful when selecting for and assembling multimers that are able to bind large target proteins with high affinity or avidity.

In certain embodiments, a selected multimer comprises more than two domains. Such multimers can be generated in a step fashion, e.g., where the addition of each new domain is tested individually and the effect of the domains is tested in a sequential fashion. In an alternative embodiment, domains are linked to form multimers comprising more than two domains and selected for binding without prior knowledge of how smaller multimers, or alternatively, how individual domains, bind.

On a larger scale, multimers (single or pools) with desired properties may be recombined to form longer multimers. In some cases variation is introduced (typically synthetically) into domains or into the linkers to form libraries. This may be achieved, e.g., with two different multimers that bind to two different targets, thereby eventually selecting a multimer with a portion that binds to one target and a portion that binds a second target.

Additional variation can be introduced by inserting linkers of different length and composition between domains. This allows for the selection of optimal linkers between multimer subunits. In some embodiments, optimal length and composition of linkers will allow for optimal binding of each domain. Domains with a particular binding affinity(s) can be linked via different linkers and optimal linkers are selected in a binding assay. For example, in certain variations, domains are selected for desired binding properties and then formed into a library comprising a variety of linkers. The library can then be screened to identify optimal linkers. Alternatively, multimer libraries can be formed where the effect of subunit or linker on target molecule binding is not known.

Accordingly, in certain embodiments, methods of the present invention include generating one or more selected multimers by providing a plurality of MSP monomer domain variants and, optionally, a plurality of other, non-MSP polypeptide domains (e.g., antigen-binding domains or non-MSP monomer domains). The plurality of MSP monomer domain variants and/or other domains is screened for binding of a desired target or mixture of targets. Members of the plurality of domains that bind the desired target or mixture of targets are identified, thereby providing domains with a desired affinity. The identified domains are joined with at least one linker to generate the multimers, wherein each multimer comprises at least two of the selected domains (at least one of which is an MSP monomer domain variant as described herein) and the at least one linker; and, the multimers are screened for an improved affinity or avidity or altered specificity for the desired target or mixture of targets as compared to the selected domains, thereby identifying the one or more selected multimers.

Multimer libraries may be generated, in some embodiments, by combining two or more libraries of monomer domains or multimers in a recombinase-based approach, where each library member comprises as recombination site (e.g., a lox site). A larger pool of molecularly diverse library members in principle harbor more variants with desired properties, such as higher target-binding affinities and functional activities. When libraries are constructed in phage vectors, which may be transformed into E. coli, library size (10⁹-10¹⁰) is limited by the transformation efficiency of E. coli. A recombinase/recombination site system (e.g., the Cre-loxP system) and in vivo recombination can be exploited to generate libraries that are not limited in size by the transformation efficiency of E. coli.

For example, the Cre-loxP system may be used to generate dimer libraries with 10¹⁰, 10¹¹, 10¹², 10¹³, or greater diversity. In some embodiments, E. coli as a host for one naive monomer library and a filamentous phage that carries a second naive monomer library are used. The library size in this case is limited only by the number of infective phage (carrying one library) and the number of infectible E. coli cells (carrying the other library). For example, infecting 1012 E. coli cells (1 L at OD600=1) with >10¹² phage could produce as many as 10¹² dimer combinations.

Selection of multimers can be accomplished using a variety of techniques including those described herein for identifying MSP monomer domain variants and/or other domains. Other selection methods include, for example, a selection based on an improved affinity or avidity or altered specificity for a target compared to selected domains. For example, a selection can be based on selective binding to specific cell types, or to a set of related cells or protein types (e.g., different virus serotypes). Optimization of the property selected for (e.g., avidity for a target) can then be achieved by recombining selected domains, as well as manipulating amino acid sequence of the individual domains or the linker region or the nucleotide sequence encoding such polypeptide regions, as discussed herein.

One method for identifying multimers can be accomplished by displaying the multimers. As with MSP monomer domain variants and other domains, multimers are optionally expressed or displayed on a variety of display systems, such as, for example, phage display, ribosome display, polysome display, nucleotide-linked display (see, e.g., U.S. Pat. Nos. 6,281,344; 6,194,550, 6,207,446, 6,214,553, and 6,258,558) and/or cell surface display, as described herein. Cell surface displays can include but are not limited to E. coli, yeast, or mammalian cells. In addition, display libraries of multimers with multiple binding sites can be panned for avidity or affinity or altered specificity for a target or for multiple targets.

MSP monomer domain variants and/or other domains, as well as multimers thereof can be screened for target binding activity in yeast cells using a two-hybrid screening assay. In this type of screen, the library to be screened is cloned into a vector that directs the formation of a fusion protein between each individual domain or multimer of the library and a yeast transcriptional activator fragment (i.e., Gal4). Sequences encoding the “target” protein are cloned into a vector that results in the production of a fusion protein between the target and the remainder of the Gal4 protein (the DNA binding domain). A third plasmid contains a reporter gene downstream of the DNA sequence of the Gal4 binding site. A library member that can bind to the target protein brings with it the Gal4 activation domain, thus reconstituting a functional Gal4 protein. This functional Gal4 protein bound to the binding site upstream of the reporter gene results in the expression of the reporter gene and selection of the MSP monomer domain variant or other polypeptide domain, or multimer thereof, as a target binding protein. (See Chien et al. Proc. Natl. Acad. Sci. USA 88:9578, 1991; Fields and Song, Nature 340: 245, 1989.) Using a two-hybrid system for library screening is further described in U.S. Pat. No. 5,811,238 (see also Silver and Hunt, Mol. Biol. Rep. 17:155, 1993; Durfee et al., Genes Dev. 7:555, 1993; Yang et al., Science 257:680, 1992; Luban et al., Cell 73:1067, 1993; Hardy et al., Genes Dev. 6:801, 1992; Bartel et al., Biotechniques 14:920, 1993; and Vojtek et al., Cell 74:205, 1993). Another useful screening system for carrying out the present invention is the E. coli/BCCP interactive screening system (Germino et al., Proc. Nat. Acad. Sci. U.S.A. 90:993, 1993; Guarente, Proc. Nat. Acad. Sci. U.S.A. 90:1639, 1993).

Other variations include the use of multiple binding compounds, such that multimers or individual domains, or libraries of these molecules, can be simultaneously screened for a multiplicity of targets that have different binding specificity. Multiple predetermined targets can be concomitantly screened in a single library, or sequential screening against a number of individual domains or multimers. In one variation, multiple targets, each encoded on a separate bead (or subset of beads), can be mixed and incubated with multimers or individual domains, or libraries of these molecules, under suitable binding conditions. The collection of beads, comprising multiple targets, can then be used to isolate, by affinity selection, selected MSP monomer domain variants or other domains, selected multimers, or library members. Generally, subsequent affinity screening rounds can include the same mixture of beads, subsets thereof, or beads containing only one or two individual targets. This approach affords efficient screening, and is compatible with laboratory automation, batch processing, and high throughput screening methods.

In another embodiment, MSP monomer domain variants or multimers can be simultaneously screened for the ability to bind multiple targets, wherein each target comprises a different label. For example, each target can be labeled with a different fluorescent label, contacted simultaneously with an MSP monomer domain variant, multimer, or library thereof. Molecules with the desired affinity are then identified (e.g., by FACS sorting) based on the presence of the labels linked to the desired labels.

Libraries of MSP monomer domain variants or multimers comprising such monomer domains (also referred in the following discussion for convenience as “affinity agents”) can be screened (i.e., panned) simultaneously against multiple targets in a number of different formats. For example, multiple targets can be screened in a simple mixture, in an array, displayed on a cell or tissue (e.g., a cell or tissue provides numerous molecules that can be bound by MSP monomer domain variants or multimers of the invention), and/or immobilized. The libraries of affinity agents can optionally be displayed on yeast or phage display systems, such as those described herein. Similarly, if desired, the protein targets (e.g., encoded in a cDNA library) can be displayed in a yeast or phage display system.

Initially, the affinity agent library is panned against the multiple targets. Optionally, the resulting “hits” are panned against the targets one or more times to enrich the resulting population of affinity agents.

If desired, the identity of the individual affinity agents and/or targets can be determined. In some embodiments, affinity agents are displayed on phage. Affinity agents identified as binding in the initial screen are divided into a first and second portion. The first portion is infected into bacteria, resulting in either plaques or bacterial colonies, depending on the type of phage used. The expressed phage are immobilized and then probed with targets displayed in phage selected as described below.

The second portion are coupled to beads or otherwise immobilized and a phage display library containing at least some of the targets in the original mixture is contacted to the immobilized second portion. Those phage that bind to the second portion are subsequently eluted and contacted to the immobilized phage described in the paragraph above. Phage-phage interactions are detected (e.g., using a monoclonal antibody specific for the ligand-expressing phage) and the resulting phage polynucleotides can be isolated.

In some embodiments, the identity of an affinity agent-target pair is determined. For example, when both the affinity agent and the target are displayed on a phage or yeast, the DNA from the pair can be isolated and sequenced. In some embodiments, polynucleotides specific for the target and affinity agent are amplified. Amplification primers for each reaction can include 5′ sequences that are complementary such that the resulting amplification products are fused, thereby forming a hybrid polynucleotide comprising a polynucleotide encoding at least a portion of the affinity agent and at least a portion of the target. The resulting hybrid can be used to probe affinity agent or target (e.g., cDNA-encoded) polynucleotide libraries to identify both affinity agent and target.

The above-described methods can be readily combined with “walking” to simultaneous generate and identify multiple multimers, each of which bind to a target in a mixture of targets. In these embodiments, a first library of affinity agents (e.g., MSP monomer domain variants or multimers) are panned against multiple targets and the eluted affinity agents are linked to the first or a second library of affinity agents to form a library of multimeric affinity agents (e.g., comprising 2, 3, 4, 5, 6, 7, 8, 9, or more domains, including one or more MSP monomer domain variants), which are subsequently panned against the multiple targets. This method can be repeated to continue to generate larger multimeric affinity agents. Increasing the number of multimer subunits may result in increased affinity and avidity for a particular target. Of course, at each stage, the panning is optionally repeated to enrich for significant binders. In some cases, walking will be facilitated by inserting recombination sites (e.g., lox sites) at the ends of MSP monomer domain variants, and optionally other domains, and recombining domain libraries by a recombinase-mediated event.

The selected multimers of the above methods can be further manipulated by, for example, recombining or shuffling the selected multimers (recombination can occur between or within multimers or both), mutating the selected multimers, and the like. This results in altered multimers that then can be screened and selected for members that have an enhanced property compared to the selected multimer, thereby producing selected altered multimers.

In view of the description herein, it is clear that the following process may be followed. MSP monomer domain variants can be formed and may be linked with one or more other MSP monomer domain variants and/or one or more other, non-MSP polypeptides (e.g., an antigen-binding domain or non-MSP monomer domain). Optionally, the domains, initially or subsequently, are selected for those sequences that are less likely to be immunogenic in the host for which they are intended. Optionally, a phage library comprising the MSP monomer domain variants or multimers is panned for a desired affinity. MSP monomer domain variants or multimers expressed by the phage may be screened for binding specificity or other activity against a target. Hetero- or homo-meric multimers may be selected. The selected polypeptides may be selected for their affinity or avidity to any target, including, e.g., hetero- or homo-multimeric targets.

Known unknown targets can be used to select the MSP monomer domain variants and/or multimers. No prior information regarding target structure is required to isolate a polypeptide comprising a MSP monomer domain variant of interest, including a multimer as described herein. The MSP monomer domain variants and/or multimers identified can have biological activity, which may include at least specific binding affinity or avidity for a selected or desired target and, in some instances, will further include the ability to block the binding of other compounds, to stimulate or inhibit metabolic pathways, to act as a signal or messenger, to stimulate or inhibit cellular activity, and the like. MSP monomer domain variants and multimers of the invention can be generated to function as ligands for receptors where the natural ligand for the receptor has not yet been identified (orphan receptors). These orphan receptor ligands can be created to either block or activate the receptor to which they bind.

A single target can be used, or optionally a variety of targets can be used, to select the MSP monomer domain variants and/or multimers. A MSP monomer domain variant, and optionally other, non-MSP domains (e.g., an antigen-binding domain or a non-MSP monomer domain), can bind a single target or a variety of targets. A multimer of the present invention can have multiple discrete binding sites for a single target, or optionally, can have multiple binding sites for a variety of targets.

The invention is further illustrated by the following non-limiting examples.

### Example 1

### Cloning of MSP94 and its Domains

MSP94 cDNA was amplified from a human prostate library by PCR using the oligonucleotide primers zc58846 (GCATCTATGTTCGTTTTTTCTATTGCTACAAACGCGTAT GCAAGTGCACAGTCATGCTATTTCATACCTAATG; SEQ ID NO:5) and zc58847 (GATCAGCTTTTGTTCGGATCCAGCGGCCGAAGCATCAGCCCGAGCGGCCGCGATTATCC ATTCACTGACAGAAC; SEQ ID NO:6). DNA encoding the mature human MSP94 protein (SEQ ID NO:4, encoded by the nucleotide sequence set forth in SEQ ID NO:3) was excised from a 1% agarose gel and purified by Qiagen gel extraction kit (Qiagen). The purified DNA was inserted into parb013 (SEQ ID NO:14) by yeast recombination followed by transformation into E. coli TG-1.

### MSP94 Domain 1 Cloning

Overlap PCR was used to generate DNA encoding Domain 1 of MSP94. Oligonucleotides zc58846 (GCATCTATGTTCGTTTTTTCTATTGCTACAAACGCGTATGCAAG TGCACAGTCATGCTATTTCATACCTAATG; SEQ ID NO:7), zc58862 (TCATGCTATTTCATAC CTAATGAGGGAGTTCCAGGAGATTCAACCAGGAAATGCATGGATCTCAAAGGAAACAAC ACCCAATAAACTCGGAGTGG; SEQ ID NO:8), zc58863 (TGTAGAAACAAGGGTGCAACAT GAAATTTCTGTTTCGTAGCAAGTGCATGTCTCACCGTTGTCAGTCTGCCACTCCGAGTTTA TTGGGTGTTTGTTTCC; SEQ ID NO:9), and zc58865 (GATCAGCTTTTGTTCGGATCCAGCGG CCGAAGCATCAGCCCGAGCGGCCGCTGTAGAAACAAGGGTGCAAC; SEQ ID NO:10) were combined and subjected to 15 rounds of PCR. DNA encoding human MSP94 domain 1 was excised from a 1% agarose gel and purified by Qiagen gel extraction kit (Qiagen). The purified DNA was inserted into parb013 by yeast recombination followed by transformation into E. coli TG-1.

### MSP94 Domain 2 Cloning

PCR was used to generate DNA encoding Domain 2 of MSP94. Oligonucleotides zc58864 (GCATCTATGTTCGTTTTTTCTATTGCTACAAACGCGTATGCAAGTGCACAGACAC CTGTGGGTTATGACAAAG; SEQ ID NO:11), zc58861 (TCTACACCTGTGGGTTATGACAAA GACAACTGCCAAAGAATCTTCAAGAAGGAGGACGGCAAGTATATCGTGGTGGAGAAGA AGGACCCAAAAAAGACCTGTTCTGTCAGTGAATGGATAATC; SEQ ID NO:12), and zc58847 (GATCAGCTTTTGTTCGGATCCAGCGGCCGAAGCATCAGCCCGAGCGGCCGCGATTATCC ATTCACTGACAGAAC; SEQ ID NO:13) were combined and subjected to 15 rounds of PCR. DNA encoding human MSP94 domain 2 was excised from a 1% agarose gel and purified by Qiagen gel extraction kit (Qiagen). The purified DNA was inserted into parb013 by yeast recombination followed by transformation into E. coli TG-1.

### Example 2

### Expression and Purification of C-terminal-His Tagged MSP94, MSP94 Domain 1, and MSP94 Domain 2

E. coli:TG1 containing parb013Msp94, parb013Msp94domain1, and parb013Msp94domain2 were cultured for 30 hrs at 30° C. in 500 mL low phosphate medium (0.351% Ammonium sulfate, 0.07% sodium citrate dehydrate, 0.1% KCl, 0.528% yeast extract, 0.528% hycase SF, 2.266% MOPS, 0.541% dextrose, 0.17% MgSO₄, pH 7.3). Cells harvested by centrifuge were resuspended in 4 mL 200 mM Tris:HCl, pH 7.4; 20% sucrose; protease inhibitor cocktail (Roche, complete) 29,000 units of Lysozyme (Ready-lyse, Epicentre) and incubated for 10 minutes at room temperature. 6 mL of water was added to the cell suspension and it was incubated on ice for an additional 15 minutes. The cells were pelleted by centrifugation and the supernatant (periplasmic extract) was transferred to a clean tube. Western blot analysis of the periplasmic extracts revealed proteins of the correct molecular weight for C-terminal His-tagged MSP94, MSP94 domain 1, and MSP94 domain 2.

The periplasmic extracts were supplements with NaCl and imidizole to the final concentrations of 25 mM Imidazole in 0.5 M NaCl, and then adjusted the pH to 7.5. The samples were loaded to a 5 mL HisTrap column and then washed with sufficient equilibration buffer to bring A_{280 nm} to baseline. Bound protein was eluted with 400 mM Imidazole (both washes include 500 mM NaCl; 50 mM NaPhos buffer at pH 7.5. Western blot analysis and coommasie stained PAGE of the eluted fractions revealed proteins of the correct molecular weight for C-terminal His-tagged Msp94, Msp94domain1, and Msp94domain2.

### Example 3

### MSP94, MSP94 Domain 1, and MSP94 Domain 2 Cloning into PhagemidXSTII

### MSP94, MSP94 Domain 1, and MSP Domain 2 PCR-derived fragments were generated and ligated into the phagemidxst2 vector (SEQ ID NO:15; the locations of various sequence features for phagemidXDSBA are shown in Table 3), as summarized below.

pARB plasmids with inserts encoding MSP94, MSP94 domain 1, and MSP94 domain 2 (pARBmsp94, pARBmsp94 D1, and pARBmsp94 D2, respectively) were constructed as described in Example 1, supra. In addition, the following oligonucleotide primers were synthesized:
zc59852 (ATGCATGGATCCTCATGCTATTTCATACCTAAT; SEQ ID NO:16)—forward primer for β-MSP domain 1 (MSP94 domain 1);
zc59852 encodes a BamHI site on the 5′ end for cloning into phagemidXST2, and can be used to amplify domain 1 or both domain 1 and 2 together (see reverse primer for β-MSP domain 2);
zc59853 (ATGCATGCGGCCGCTTGTAGAAACAAGGGTGCAACAT GA; SEQ ID NO:17)—reverse primer for β-MSP domain 1 (MSP94 domain 1); zc59853 encodes a NotI site on the 3′ end for cloning into phagemidXST2;
zc59883 (ATGCATGGATCCTCTACACCTGTGGGTTATGACAAA; SEQ ID NO:18)—forward primer for β-MSP domain 2 (MSP94 domain 2);
zc59883 encodes a BamHI site on the 5′ end for cloning into phagemid X ST2;
zc59854 (ATGCATGCGGCCGCTGATTATCCATTCACTGACAGA; SEQ ID NO:19)—reverse primer for β-MSP domain 2 (MSP94 domain 2);
zc59854 encodes a NotI site on the 3′ end for cloning into phagemidXST2, and can be used to amplify domain 2 or both domain 1 and 2 together (see forward Primer for β-MSP domain 1).

The following PCR reactions were set up utilizing Invitrogen Accuprime polymerase and buffers (cat# 12344-024):

Fragment MSP94 D1
Template: pARBmsp94 D1
Oligos: zc59852, zc59853

Fragment MSP94 D2
Template: pARBmsp94 D2
Oligos: zc59883, zc59854

Fragment MSP94
Template: pARBmsp94
Oligos: zc59852, zc59854

The above reactions were run in parallel using the following thermocycle profile: 1 cycle-95° C. for 5 min.; 25 cycles-95° C. for 15 sec., 55° C. for 30 sec., 68° C. for 1 min.; 1 cycle-72° C. for 7 min., 4° C. hold. PCR products were run on 1% TAE gels in the presence of EthBr and the specific bands excised. The fragments were gel extracted utilizing the Qiagen Gel Extraction kit and eluted in H₂O.

Each of the fragments was digested with NotI and BamHI restriction enzymes (New England Biolabs). The phagemidXST2 positive sequence vector was also digested with NotI and BamHI restriction enzymes in parallel to the PCR fragments. The digested fragments and vector were run on 1% TAE gels in the presence of EthBr and the specific bands excised. The fragments and vector were gel extracted utilizing the Qiagen Gel Extraction kit and eluted in the kit's EB buffer.

In separate reactions, each of the three fragments was reacted together with the cut phagemidXST2 vector in the presence of ligase, overnight at ˜15° C. using Invitrogen T4 DNA ligase and supplied buffer (cat# 15224-017). The overnight ligation was electroporated into Invitrogen Electro-competent DH12s Bacterial strain and plated on Amp 100 μg/ml plates.

Colonies harboring plasmid with the correct sequence for Phagemidxst2-Msp94 (full-length, domains 1 and 2), Phagemidxst2-Msp94Domain1 (with C37G substitution), and Phagemidxst2-Msp94Domain2 (with C73G substitution) were identified via sequence analysis, and extracted positive plasmids were stored at −20° C.

TABLE 3
Location of Sequence Features of PhagemidXST2

| | Sequence Location |
|---|---|
| | (nucleotide residue |
| | positions of |
| Sequence Identification (component name) | SEQ ID NO: 15) |
| EcoRI-Restriction Enzyme site DNA linker | 1-6 |
| PUC Ori (E. coli origin of replication) | 7-844 |
| B-Lactamase with stop codon (amp resistence) | 845-1705 |
| F1 Ori - (filamentous phage origin of | 2142-2595 |
| replication) | |
| Lac operon | 2600-2758 |
| HindIII - Restriction Enzyme site DNA linker | 2762-2767 |
| PhoA promoter upstream seq (which may | 2768-2942 |
| include expression enhancer region) | |
| PhoA promoter and ribosome binding site | 2943-3043 |
| ST2 leader sequence | 3044-3112 |
| BamHI, KpnI and NotI Restriction Enzyme | 3113-3132 |
| sites - Multiple cloning site for inserts | |
| Six His tag sequence | 3133-3150 |
| MluI site | 3151-3156 |
| “GSGG” flexible linker | 3157-3168 |

### Example 4

### Expression of MSP94, MSP94 Domain 1, and MSP Domain 2 pGIII Fusions from PhagemidXST2

A. Expression of the pGIII Fusions in Whole Cell Lysates

Positive DH12s strain clones carrying sequence for constructs Phagemidxst2-Msp94 (full-length, domains 1 and 2), Phagemidxst2-Msp94Domain1(C37G), and Phagemidxst2-Msp94Domain2(C73G) were used to seed 2 ml cultures of Phosphate Limiting Medium (0.027M (NH₄)₂SO₄, 0.002414M Sodium citrate dehydrate, 0.014353M KCl, 0.536% (w/v) Yeast extract, 0.536% (w/v) Hycase SF, 0.11M MOPS, 0.03053M Dextrose, 0.00702M MgSO4 heptahydrate, pH (adjusted) 7.3±0.2, filtration 0.22 μm or 0.1 μm, specific gravity 1.016) with Amp and allowed to proceed for 36 hrs at 250 rpm, 37° C.

The bacteria from the cultures were pelleted and the supernatant was saved. The pellet was resuspended in 250 μl of 4× loading buffer (Nu-page-Invitrogen). 15 μl of this resuspension was aliquoted and 2 μl of 10× reducing agent (Nu-page-Invitrogen) was added to this volume and used for gel analysis. Samples were incubated for 15 min at 75° C., loaded, and run on a 4-12% Bis-Tris Nu-Page Invitrogen gel in MES buffer. The proteins were transferred to nitrocellulose, blocked 2 hrs with 2.5% non-fat dry milk in western A buffer and incubated overnight in a 1:2000 dilution of anti-His HRP conjugated antibody (R&D Systems) also in 2.5% non-fat dry milk in western A buffer solution. The blot was then rinsed 3× in western A buffer, reacted with Pierce Pico reagent and analyzed on the Image Quant apparatus and software. Correct M.W. band signals were observed in the Phagemidxst2-Msp94 (full-length, domains 1 and 2), Phagemidxst2-Msp94Domain1(C37G), and Phagemidxst2-Msp94Domain2(C73G) lanes.

B. Expression of the pGIII Fusions on Phage

Single colonies of TG1 bacterial strain carrying phagemid for constructs Phagemidxst2-Msp94 (full-length, domains 1 and 2), Phagemidxst2-Msp94Domain1(C37G), and Phagemidxst2-Msp94Domain2(C73G) were used to seed 2 ml cultures of Phosphate Limiting Medium with Amp and allowed to proceed for 36 hrs at 250 rpm, 30° C.

After culturing for 36 hrs, the OD₆₀₀ reading was taken and the cultures diluted to 0.1 OD in 40 mls of fresh Phosphate Limiting Medium with Amp in a shake flask and 400 μl of M13K07 helper phage was added to the cultures (Invitrogen). The temperature was raised to 37° C. for 6 hours, then dropped to 30° C. and the cultures were allowed to proceed overnight at 250 rpm. The cultures were then collected, centrifuged at 8 kG for 20 minutes, and the supernatant saved. 8 ml of 20% PEG 8000, 2.5M NaCL was added to the supernatant and mixed. The phage were allowed to precipitate overnight at 4° C. The phage were then pelleted by centrifugation for 15 minutes at 10 kG and the supernatant was aspirated. The phage pellet was resuspended in 250 μl of PBS.

The resuspended phage samples were examined by Western analysis. To 35 μl of phage resuspension, 12.5 μl of 4× loading buffer (Nu-page-Invitrogen) and 5 μl of 10× reducing agent (Nu-page-Invitrogen) was added to this volume. Samples were incubated for 10 minutes at 70° C., loaded, and run on a 4-12% Bis-Tris Nu-Page Invitrogen gel in MES Buffer. The proteins were transferred to nitrocellulose, blocked 2 hrs with 2.5% non-fat dry milk in western A buffer and incubated approximately 96 hrs in a 1:2000 dilution of anti-His HRP conjugated antibody (R&D Systems) also in 2.5% non-fat dry milk in western A buffer solution. The blot was then rinsed 3× in western A buffer, reacted with Pierce Pico reagent and analyzed on the Image Quant apparatus and software. Correct M.W. band signals were observed in the Phagemidxst2-Msp94, Phagemidxst2-Msp94Domain1(C37G), and Phagemidxst2-Msp94Domain2(C73G) lanes.

### Example 5

### Expression of His-Tagged MSP94, MSP94 Domain 1, and MSP94 Domain 2 pGIII Fusions from PhagemidXSTII

E. coli:TG1 containing PhagemidXSTII-Msp94Domain1, PhagemidXSTIIMsp94-Domain2, or PhagemidXSTII-Msp94 were cultured for 30 hrs at 30° C. in 500 mL low phosphate medium (0.351% Ammonium sulfate, 0.07% sodium citrate dehydrate, 0.1% KCl, 0.528% yeast extract, 0.528% hycase SF, 2.266% MOPS, 0.541% dextrose, 0.17% MgSO₄, pH 7.3). Cells harvested by centrifuge were resuspended in 4 mL 200 mM Tris:HCl, pH 7.4; 20% sucrose; protease inhibitor cocktail (Roche, complete) 29,000 units of Lysozyme (Ready-lyse, Epicentre) and incubated for 10 minutes at room temperature. 6 mL of water was added to the cell suspension and it was incubated on ice for an additional 15 minutes. The cells were pelleted by centrifugation and the supernatant (periplasmic extract) was transferred to a clean tube. Western blot analysis of the periplasmic extracts revealed proteins of the correct molecular weight for His-tagged Msp94, Msp94domain1, and Msp94domain2 fused to M13 pGIII.

### Example 6

### Conversion of PhagemidXSTII to Expression Constructs for MSP94, MSP94 Domain 1, and MSP94 Domain 2

PhagemidXSTII-Msp94Domain1, PhagemidXSTII-Msp94Domain2, and PhagemidXSTII-Msp94 were cut with the restriction enzyme MluI at 37° C. for 4 hours. The digested DNA was run on a 1% agarose gel and the DNA representing cut plasmid was excised form the gel and purified using a Qiagen gel extraction kit as described by the manufacturer (Qiagen). Approximately 5 ng of plasmid DNA was religated overnight at 15° C., using T4-DNA ligase in a 30 ml reaction. 1 ml of each ligation reaction was used to transform E. coli TG-1 by electroporation and then plated on LBamp plates. Sequencing of individual clones revealed plasmid sequences that encoded soluble C-terminal His-tagged MSP94, MSP94 domain 1, or MSP94 domain 2 (MSP94-CHis, MSP94D1-CHis, or MSP94D2-CHis). The nucleotide coding sequences for MSP94-CHis, MSP94D1-CHis, and MSP94D2-CHis are shown in SEQ ID NOs: 20, 22, and 24, respectively; and the corresponding amino acid sequences are shown in SEQ ID NOs: 21, 23, and 25, respectively. The first 25 amino acids of each of SEQ ID NOs: 21, 23, and 25 correspond to a secretory signal sequence, thereby yielding a mature protein start site at position 26 for each sequence.

### Example 7

### Expression of C-terminal His-tagged MSP94, MSP94 Domain 1, and MSP94 Domain 2 from PhagemidXSTII

E. coli:TG1 containing PhagemidXSTIIMluI-Msp94Domain 1, PhagemidXSTIIMluI-Msp94Domain2, or PhagemidXSTIIMluI-Msp94 were cultured for 30 hrs at 30° C. in 500 mL low phosphate medium (0.351% Ammonium sulfate, 0.07% sodium citrate dehydrate, 0.1% KCl, 0.528% yeast extract, 0.528% hycase SF, 2.266% MOPS, 0.541% dextrose, 0.17% MgSO₄, pH 7.3). Cells harvested by centrifuge were resuspended in 4 mL 200 mM Tris:HCl, pH 7.4; 20% sucrose; protease inhibitor cocktail (Roche, complete) 29,000 units of Lysozyme (Ready-lyse, Epicentre) and incubated for 10 minutes at room temperature. 6 mL of water was added to the cell suspension and it was incubated on ice for an additional 15 minutes. The cells were pelleted by centrifugation and the supernatant (periplasmic extract) was transferred to a clean tube. Western blot analysis of the periplasmic extracts revealed proteins of the correct molecular weight for C-terminal His-tagged MSP94, MSP94 domain 1, and MSP94 domain 2.

### Example 8

### Expression of His-tagged MSP94, MSP94 Domain 1, and MSP94 Domain 2 pGIII fusions from PhagemidXDSBA

DNA fragments encoding MSP94, MSP94 Domain 1, and MSP Domain 2 were ligated into the phagemidXDSBA vector (SEQ ID NO:26; the locations of various sequence features for phagemidXDSBA are shown below in Table 4) to generate PhagemidXDSBA-Msp94, PhagemidXDSBA-Msp94Domain1, and PhagemidXDSBA-Msp94Domain2 vector constructs. E. coli:TG1 containing PhagemidXDSBA-Msp94, PhagemidXDSBA-Msp94Domain1, or PhagemidXDSBA-Msp94Domain2 were cultured for 30 hrs at 30° C. in 500 mL low phosphate medium (0.351% Ammonium sulfate, 0.07% sodium citrate dehydrate, 0.1% KCl, 0.528% yeast extract, 0.528% hycase SF, 2.266% MOPS, 0.541% dextrose, 0.17% MgSO₄, pH 7.3). Cells harvested by centrifuge were resuspended in 4 mL 200 mM Tris:HCl, pH 7.4; 20% sucrose; protease inhibitor cocktail (Roche, complete) 29,000 units of Lysozyme (Ready-lyse, Epicentre) and incubated for 10 minutes at room temperature. 6 mL of water was added to the cell suspension and it was incubated on ice for an additional 15 minutes. The cells were pelleted by centrifugation and the supernatant (periplasmic extract) was transferred to a clean tube. Western blot analysis of the periplasmic extracts revealed proteins of the correct molecular weight for His-tagged MSP94, MSP94 domain 1, and MSP94 domain 2 fused to M13 pGIII.

TABLE 4
Location of Sequence Features of PhagemidXDSBA

| | Sequence Location |
|---|---|
| | (nucleotide residue |
| | positions of |
| Sequence Identification (component name) | SEQ ID NO: 26) |
| EcoRI-Restriction Enzyme site DNA linker | 1-6 |
| PUC Ori (E. coli origin of replication) | 7-844 |
| β-Lactamase with stop codon (amp resistence) | 845-1705 |
| F1 Ori - (filamentous phage origin of | 2142-2595 |
| replication) | |
| Lac operon | 2600-2758 |
| HindIII - Restriction Enzyme site DNA linker | 2762-2767 |
| PhoA promoter upstream seq (which may | 2768-2942 |
| include expression enhancer region) | |
| PhoA promoter and ribosome binding site | 2943-3042 |
| DSBA leader sequence | 3043-3099 |
| BamHI, KpnI and NotI Restriction Enzyme | 3100-3119 |
| sites - Multiple cloning site for inserts | |
| Six His tag sequence | 3120-3137 |
| MluI site | 3138-3143 |
| “GSGG” flexible linker | 3144-3155 |

### Example 9

### Conversion of PhagemidXDSBA to Expression Constructs For MSP94, MSP94 Domain 1, and MSP94 Domain 2

PhagemidXDSBA-Msp94Domain1, PhagemidXDSBA-Msp94Domain2, and PhagemidXDSBA-Msp94 were cut with the restriction enzyme MluI at 37° C. for 4 hours. The digested DNA was run on a 1% agarose gel and the DNA representing cut plasmid was excised form the gel and purified using a Qiagen gel extraction kit as described by the manufacturer (Qiagen). Approximately 5 ng of plasmid DNA was religated overnight at 15° C., using T4-DNA ligase in a 30 ml reaction. 1 ml of each ligation reaction was used to transform E. coli TG-1 by electroporation and then plated on LBamp plates. Sequencing of individual clones revealed plasmid sequences that encoded soluble C-terminal His-tagged MSP94 domain 1, MSP94 domain2, or MSP94.

### Example 10

### Expression of C-terminal His-tagged MSP94, MSP94 Domain 1, and MSP94 Domain2 from PhagemidXDSBA

E. coli:TG1 containing PhagemidXDSBAMluI-Msp94Domain1, PhagemidXDSBAMluI-Msp94Domain2, or PhagemidXDSBAMluI-Msp94 were cultured for 30 hrs at 30° C. in 500 mL low phosphate medium (0.351% Ammonium sulfate, 0.07% sodium citrate dehydrate, 0.1% KCl, 0.528% yeast extract, 0.528% hycase SF, 2.266% MOPS, 0.541% dextrose, 0.17% MgSO₄, pH 7.3). Cells harvested by centrifuge were resuspended in 4 mL 200 mM Tris:HCl, pH 7.4; 20% sucrose; protease inhibitor cocktail (Roche, complete) 29,000 units of Lysozyme (Ready-lyse, Epicentre) and incubated for 10 minutes at room temperature. 6 mL of water was added to the cell suspension and it was incubated on ice for an additional 15 minutes. The cells were pelleted by centrifugation and the supernatant (periplasmic extract) was transferred to a clean tube. Western blot analysis of the periplasmic extracts revealed proteins of the correct molecular weight for C-terminal His-tagged MSP94, MSP94 domain 1, and MSP94 domain 2.

### Example 11

### Phage-ELISA for MSP94, MSP94 Domain 1, and MSP94 Domain 2

A Nunc Maxisorb 96 well flat bottom plate was obtained. The Abcam anti-PSP (anti-MSP94) antibody (ab19070) was diluted to 1 μg/ml in 0.1M Carbonate pH 9.6 in a 3 ml volume. The Mu IgG Isotype control antibody (R&D systems, cat #MAB002) was diluted to 1 μg/ml in 0.1M Carbonate pH 9.6 in a 3 ml volume. For each antibody solution, 100 μl volumes were added to two 12 well rows of the plate. The plate was incubated, covered overnight at 4° C.

After overnight incubation, the plate was emptied by flick inversion and blotted on paper towels. 250 μl per well of PBS 0.1% Tween 20 (PBS-T) with 2.5% non-fat dry milk (blocking buffer) was added for 1 hr at 37° C. to block. The blocked plate was washed 3× with PBS-T.

Phage stock solutions were created by diluting stocks (as noted in Example 4, section B, supra) 1:40 in blocking buffer. A 1:400 stock was also created in blocking buffer (500 μl vol. of each dilution). A purified, full-length MSP94 (SEQ ID NO:4) protein sample was obtained (2.55 mg/ml). 1 μg/ml and 100 ng/ml dilutions were performed in blocking buffer.

100 μl of each dilute phage stock solution was added to wells in duplicate for each primary antibody noted above such that for each stock there were two wells with 100 μl of 1:40 as well as two wells with 100 μl of 1:400 dilution for both anti-PSP and MuIgG1 sets. One hundred μl of purified full-length MSP94 protein sample solutions were added to wells in duplicate for each primary antibody noted above such that for each protein dilution there were two wells with 100 ng, as well as two wells with 10 ng for both anti-PSP and MuIgG1 sets. All wells were incubated for 1 hr at room temperature. The plate was washed 5× with PBS-T.

100 μl of a 1:5000 dilution in blocking buffer of an anti-M13 antibody (Amersham 27-9421-01) was added to the phage specific sample wells. A 100 μl volume of a 1:1000 dilution in blocking buffer of an anti-His antibody was added to the purified protein specific sample wells. The plate was incubated for 2.5 hrs at room temperature and then washed 7× with PBS-T.

100 μl of TMB substrate was added to each well and after approximately 3-5 min. 50 μl of TMB stop solution was added to each well to stop the reaction. The plate was read on a microplate reader at 450 nM wavelength. The ELISA results indicated the following signals:
approximately 0.05 for background;
0.427 for the phagemidxst2-Msp94 (full-length, domains 1 and 2) phage sample at 2.5 ul/well;
0.095 for the phagemidxst2-Msp94 (full-length, domains 1 and 2) phage sample at 0.25 ul/well;
approximately that of background for the phagemidxst2-Msp94Domain1(C37G) and phagemidxst2-Msp94Domain2(C73G) phage samples at 2.5 or 0.25 μl/well;
0.231 for the purified full-length MSP94 protein sample at 100 ng/well;
0.06 for the purified full-length MSP94 protein sample at 10 ng/well.

### Example 12

### Library Construction

MSP is structurally conserved across species without strong primary sequence conservation. (See Wang et al., J. Mol. Biol., 346:1071-1082, 2005; Ghasriani et al., J. Mol. Biol, 362:502-515, 2006; Wang et al., Comparative Biochemistry and Physiology, 142:251-257, 2005.) NMR analysis shows that MSP94 contains two structural domains, D1 and D2, comprised of β sheets (β1-β10, Wang et al., J. Mol. Biol, 346:1071-1082, 2005) linked by unstructured loops held together by peptide and disulfide bonds. Based on the sequence of human MSP (human MSP94), the unstructured loop (loop I) between β1 and β2 in D1 contains 10 amino acids that are variable between species; and the unstructured loop (loop II) between β5 and β6 in D1 contains 5 amino acids that are variable between species. In addition, based on the sequence of human MSP94, the unstructured loop (loop III) between β7 and β8 in D2 contains 7 amino acids that are variable between species; and the unstructured loop (loop IV) between β9 and β10 in D2 contains 8 amino acids that are variable between species. Synthetic D1 and D2 libraries were constructed comprising genes encoding D1 with variable loops I and II, and D2 with variable loops III and IV. In these examples, the sequence diversity was four-fold at each position incorporating the amino acids alanine, aspartic acid, serine, and tyrosine in equal proportions. Although four-fold diversity was chosen for these examples, any degree of sequence diversity can easily be incorporated at any position because the libraries are totally synthetic.

To achieve the sequence diversity in the protein, redundant deoxyribooligonucleotides were synthesized and used with overlap PCR and amplification to construct the encoding genes. Cysteine 37 and cysteine 73 were changed to glysine to avoid unpaired cysteines within D1 and D2 domain libraries. Cysteine 37 and cysteine 73 were retained in the full length libraries.

The following oligos were synthesized and used to generate DNA fragments encoding MSP94 D1 with variable loops I and II, and fragments encoding MSP94 D2 with variable loops III and IV:

zc59201 - sense msp94D1 YADS;
(TCATGCTATTTCATACCTAATKMYKMYKMYKMYKMYKMYKMYKMYKMYK
MYTGCATGGATCTCAAAGGAAACAAACACCCAATAAACTCGGAGTGG;
SEQ ID NO:27)
zc59202 - anti-sense msp94D1 YADS;
(TGTAGAAACAAGGGTGCAACATGARKMRKMRKMRKMRKMGCAAGTGATG
TCTCACCGTTGTCAGTCTGCCACTCCGAGTTTATTGGGTGTTTGTTTCC;
SEQ ID NO:28)
zc59859 - sense msp94d2 YADS;
(TATGCAGGATCCTCTACACCTKMYKMYKMYKMYKMYKMYKMYTGCCAAA
GAATCTTCAAGAAGGAGGACGGCAAGTATATCGTG; SEQ ID NO:29)
zc59204 - anti-sense msp94d2 YADS;
(GATTATCCATTCACTGACAGAACAGGTRKMRKMRKMRKMRKMRKMRKMR
KMCACGATATACTTGCCGTCCTCCTTC; SEQ ID NO:30)
zc60488 - 3′ reverse oligo for amplification of
MSP94 D1 or D1 YADS and incorporation of NotI site
compatible with phagemidX;
(ATAAGAATATGCATGCGGCCGCTTGTAGAAACAAGGGTGCAACATGA;
SEQ ID NO:31)
zc59857 - forward oligo for MSP94D2 YADS amplifi-
caiton and insertion into phagemid X;
(TATGCAGGATCCTCTACACCT (SEQ ID NO:32)
zc60489 - 3′ reverse oligo for amplification or
MSP94 D2 YADS including a NotI site compatible
with phagemid X and GIII fusion.
(ATAAGAATATGCATGCGGCCGCTGATTATCCATTCACTGACAGA;
SEQ ID NO:33)

The following PCR reactions were set up-utilizing AdvantageII polymerase and buffers. The primers were annealed to each other and extended to create a core double stranded fragment.

MSP94D1YADS: Overlap PCR was used to generate DNA encoding MSP94D1YADS (SEQ ID NO:35, encoded by the nucleotide sequence set forth in SEQ ID NO:34). Oligonucleotides zc59201, zc59202, zc59857 and zc60489 were combined and subjected to 15 rounds of PCR. DNA encoding human MSP94D1YADS was excised from a 1% agarose gel and purified by Qiagen gel extraction kit (Qiagen).

MSP94D2YADS: Overlap PCR was used to generate DNA encoding MSP94D2YADS (SEQ ID NO:37, encoded by the nucleotide sequence set forth in SEQ ID NO:36). Oligonucleotiedes zc59859, zc59204, zc59857 and zc60489 were combined and subjected to 15 rounds of PCR. DNA encoding human MSP94D2YADS was excised from a 1% agarose gel and purified by Qiagen gel extraction kit (Qiagen).

Purified MSP94D1YADS and MSP94D2YADS PCR products were cut overnight, at 37° C. with BamHI and NotI restriction enzyme (New England Biolabs). The digested PCR products were purified from a 1% agarose gel using the Qiagen Gel Extraction Kit (Qiagen).

Fifty micrograms of a large purified prep of phagemidxst2 was digested at 37° C. O/N with BamHI and NotI (New England Biolabs). The digested phagemid vector was purified from a 1% agarose gels using the Qiagen Gel Extraction kit (Qiagen).

Ligations were set up to target of 1×10⁹ clonal diversity for each of MSP94D1 YADS and MSP94D2 YADS libraries using purified BamHI and HindIII cut PCR products fragments and BamHI and HindIII cut phagemid vector. 5 μgs MSP94D1 YADS fragment was combined with 10 μgs vector in 1 ml volume. 3 μg MSP94D2YADS fragment was combined with 6 μg vector in 600 μl volume. Ligation reactions were incubated with 1 unit of T4 DNA ligase for each mg of vector DNA at 15° C. for 16 hours.

Ligations were ethanol precipitated resuspended in 10 μl of H₂O and mixed with 480 μl of DH12s electro competent cells (Invitrogen). The cells were subjected to electroporation, 60 ml of the suspension in each 1 mm cuvettes using the 2.5 kV, 25 uF, and 200 ohms. After each electroporation, the bacterial in the cuvette was resuspended in 1 ml of SOC media and transferred to a tube and outgrown at 37° C. for 1 hr. Library titer showed a calculated diversity of 2×10⁸ for MSP94D YADS and 1×10⁸ for MSP94D2 YADS libraries.

The pooled cells from the electroporation outgrowth were centrifuged for 10 min at 2000 rpm on a tabletop centrifuge and the supernatant removed. The pellets were then resuspended in 24 mls of Low Phosphate media (0.351% Ammonium sulfate, 0.07% sodium citrate dehydrate, 0.1% KCl, 0.528% yeast extract, 0.528% hycase SF, 2.266% MOPS, 0.541% dextrose, 0.17% MgSO₄, pH 7.3) supplemented with ampicillin to a final concentration of 100 μg/ml and the cultures incubated at 37° C. for 2 hours. After 2 hours, 3 mls of M13KO7 helper phage (Invitrogen) were added to each of the cultures, mixed and then transferred to a 2 L baffled shake flask containing 293 mls of 37° C. pre-heated low phosphate media supplemented with ampicillin to a final concentration of 100 μg/ml. This culture was incubated for an additional 2 hours at 37° C. After 2 hours kanamycin was added to a final concentration of 70 μg/ml and the cultures were incubated overnight on an orbital shaker, shaking at 250 RPM, at 30° C.

The next day, the culture was centrifuged for 20 min at 8000XG to pellet bacteria. The supernatant was transferred to a new tube containing 62 mls of 20% (W/V) PEG 8000, 2.5M NaCl. The solutions were mixed and allowed to incubate in ice for 2 hrs. The phage were pelleted by centrifugation at 8000XG for 20 minutes. The supernatant was discarded and the phage resuspended in 5 mL of PBS.

The phage titers were calculated to be 1×10¹³ cfu/ml for MSP94 D1 YADS and 2×10¹² cfu/ml for MSP94 D2 YADS.

### Example 13

### MSP94 Phase Enrichment

The phagemidxst2 system was tested for positive enrichment as described below.

To prepare for an enrichment experiment, a pooled sample of MSP94 domain 1 and domain 2 YADS library phage and the phagemidxst2-Msp94 (full-length, domains 1 and 2) phage aliquot (noted in Example 4, section B, supra) were titered as follows:

The day previous to the titer, 2.5 ml of low phosphate media without antibiotics was inoculated with the TG1 bacterial strain and allowed to proceed overnight at 37° C., 250 RPM. On the day of titer, 25 ml of low phosphate media without antibiotics is prewarmed to 37° C. and seeded with 200 μl of the overnight culture and allowed to proceed to an OD₆₀₀ of 0.5 at 37° C., 250 RPM.

20 μl of each phage aliquot was diluted into 980 μl of Low Phosphate media and mixed in eppendorf tubes. 10-fold dilutions were then performed serially in final volumes of 50 μl such that at the end of the titer there was a 1:100 through a 1:10¹¹ serial dilutions in ten-fold increments. The dilutions were then incubated at 37° C. for approximately 15 min. 45 μl of a TG1 bacterial strain culture, seeded approximately 2 hrs earlier and allowed to reach an OD₆₀₀ of 0.5, was then added to 45 μl of each titer point and briefly mixed. The mixture was then incubated at 37° C. for 30 min. and briefly mixed, and 10 μl of each point was plated and marked in a drop on a 100 μg/ml Amp plate. The plates were incubated overnight and the number of colonies counted at each titer point. The numbers were then converted to a CFU/ML format. The titers of both phage aliquots were at 4×10¹¹ CFU/ml.

The day previous to the enrichment panning, two sets of Nunc Immuno tubes were incubated with antibody. Two mls of PBS with 15 μg/ml of Abcam anti-PSP antibody (ab19070) was added to each of three tubes. Additionally, two mls of PBS with 15 μg/ml of Mu IgG Isotype control antibody (R&D systems, cat #MAB002) was added to each of three tubes. The tubes were capped and tumbled slowly overnight at 4° C.

The day previous to the phage panning, 2.5 ml of low phosphate media without antibiotics was inoculated with the TG1 bacterial strain and allowed to proceed overnight at 37° C., 250 RPM. The next morning, the anti-PSP antibody and the Mu IgG Isotype control antibody coated tubes were emptied, washed 5× with PBS 0.1% Tween 20 (PBS-T) and filled with 3 mls of PBS 0.1% Tween 20 (PBS-T) with 2% non-fat dry milk (blocking buffer). The tubes were capped and tumbled slowly at room temperature for 1 to 2 hrs.

To begin the enrichment experiment, the phagemidxst2-Msp94 (full-length, domains 1 and 2) phage was diluted serially ten-fold in PBS in 70 μl volumes out to a final concentration of 4×10⁴ phage/ml. 450 μl of the pooled sample of MSP94 domain 1 and domain 2 YADS library phage at 4×10¹¹ phage/ml was aliquoted into three tubes each. 50 μl of the 4×10⁸, 4×10⁶ and 4×10⁴ phage/ml dilutions of the phagemidxst2-Msp94 (full-length, domains 1 and 2) noted above were added separately to the 450 μl of the other phage to create 500 μl final volume aliquots. Each of the three aliquots were panned separately as described below.

Into three separate, labeled Nunc Immuno tubes, the following were added: 500 μl of PBS 0.1% Tween 20 (PBS-T) with 4% non-fat dry milk, 500 μl of the phage mixtures and 1 ml of PBS 0.1% Tween 20 (PBS-T) with 2% non-fat dry milk was added. The tubes were capped and tumbled slowly at room temperature for 1 hr.

After 1 hour of blocking, the Mu IgG Isotype control antibody coated tubes were emptied, labeled and the blocked phage mixtures were added. The tubes were capped and tumbled slowly at room temperature for an additional 1 hr.

After 1 hour of incubation, the anti-PSP antibody coated tubes were emptied of blocking buffer and labeled, and the phage mixtures from the Mu IgG Isotype control antibody coated tubes were transferred to them. The tubes were capped and tumbled slowly at room temperature for an additional 1 hr.

After 1 hour of incubation, the phage mixtures were discarded, the tubes washed 15× with PBS 0.1% Tween 20 (PBS-T) and then 2× with PBS. The target-bound phage were eluted by the addition of 1 ml of 100 mM Triethyleneamine to the washed tubes. The tubes were capped and tumbled slowly at room temperature for 10 min. The eluted phage were then transferred to labeled Eppendorf tubes with 0.5 ml of 1M TRIS pH 7.4 for neutralization. A 50 μl sample was removed from each sample and titered as described above.

The TG1 culture that was started the day previous was utilized to start a larger 25 ml culture in low phosphate media without antibiotics at 0.03 OD₆₀₀. The culture was allowed to proceed in a baffled flask at 37° C., 250 RPM to an OD of 0.5. This culture was then utilized for phage titer and rescue.

The rest of the output phage volume was each separately added to 5 mls of 0.5 OD₆₀₀ TG1 culture, mixed, and incubated without shaking at 37° C. for 30 min. After 30 min., 250 μl of M13KO7 helper phage (Invitrogen, >1×10¹¹ pfu/ml) was added to each tube, mixed and incubated without shaking at 37° C. for 30 min. The infected cultures were then each added to 30° C. pre-warmed 25 ml of low phosphate media containing 100 μg/ml Ampicillin in a baffled flask and incubated overnight at 30° C., 250 RPM.

The next morning, the cultures were centrifuged at 8 kG for 20 min. to pellet bacteria. The supernatant (˜30 mls) was transferred to a new tube and the phage precipitated by addition of 6 mls of 20% (W/V) PEG 8000, 2.5M NaCl. The solutions were mixed and allowed to incubate in ice for approximately 2 hrs. The precipitated phage were each pelleted by centrifugation at 8 kG for 20 minutes and resuspended in 1 ml of PBS and titered.

A second round of panning using the first round amplified, precipitated phage stocks was performed as above. The second round pan was performed over coated tubes pre-incubated with 15 μg/ml Mu IgG Isotype control antibody, while the concentration of anti-PSP antibody pre incubation was dropped to 7 μg/ml.

TG1 strain bacterial colonies infected with titered, amplified phage from the second round were analyzed by PCR for the presence of phagemidxst2-Msp94 (full-length, domains 1 and 2) sequence insert. The PCR utilized Supermix (Invitrogen) reagent and oligos zc59852 and zc59854 (SEQ ID NOs: 23 and 26; see Example 3, supra). A small amount of the bacterial colonies was utilized as template in the reaction. 20 colonies were examined for each initial level of phagemidxst2-Msp94 input phage. The following thermocycle profile was utilized: 1 cycle-94° C. for 5 min.; 25 cycles-94° C. for 30 sec., 55° C. for 30 sec., 72° C. for 30 sec.; 1 cycle-72° C. for 7 min., 4° C. hold. Reactions were run on a Agarose gel and the presence of a correct MW band observed.

Results of the second round amplified phage stock PCR colony analysis are summarized in Table 3, below.

TABLE 5
Results of 2nd Round Amplified Phage Stock
PCR Colony Analysis for PhagemidX-Msp94

| Initial input | |
|---|---|
| phagemidxst2- | |
| Msp94 (cfu) | Positive colonies |
| 2 × 10⁷ | ~14 of 20 |
| 2 × 10⁵ | ~10 of 20 |
| 2 × 10³ | ~1 of 20 |

A third round of panning using the second round amplified, precipitated phage stocks was performed only on the 2×10³ cfu initial input phage subset as above. The third round pan was performed over coated tubes pre-incubated with 15 μg/ml Mu IgG Isotype control antibody, while the concentration of anti-PSP antibody pre-incubation was 7 μg/ml. TG1 strain bacterial colonies infected with titered, amplified phage from the third round were analyzed by PCR for the presence of phagemidxst2-Msp94 (full-length, domains 1 and 2) sequence insert as above. Results of the third round amplified phage stock PCR colony analysis showed that approximately 10 of 20 colonies were positive.

The results demonstrated that after three rounds of panning, utilizing the described phagemid system, panning and amplification techniques, specific sequence phage can be enriched to over half the phage population, over an initial background of 1×10⁸ to 1.

### Example 14

### Construction of Larger, More Diverse Libraries

A larger library for MSP94 domain 1 YADS and MSP94 domain 2 YADS was created to ensure greater, directed sequence diversity within the domain variable regions.

The following oligos were synthesized to facilitate clonable fragment generation via PCR:

zc59201 - sense msp94D1 YADS;
(TCATGCTATTTCATACCTAATKMYKMYKMYKMYKMYKMYKMYKMYKMYK
MYTGCATGGATCTCAAAGGAAACAAACACCCAATAAACTCGGAGTGG;
(SEQ ID NO:38)
zc59202 - anti-sense msp94D1 YADS;
(TGTAGAAACAAGGGTGCAACATGARKMRKMRKMRKMRKMGCAAGTGCAT
GTCTCACCGTTGTCAGTCTGCCACTCCGAGTTTATTGGGTGTTTGTTT
CC; SEQ ID NO:39)
zc59859 - sense msp94D2 YADS;
(TATGCAGGATCCTCTACACCTKMYKMYKMYKMYKMYKMYKMYTGCCAAA
GAATCTTCAAGAAGGAGGACGGCAAGTATATCGTG; SEQ ID NO:40)
zc59204 - anti-sense msp94D2 YADS;
(GATTATCCATTCACTGACAGAACAGGTRKMRKMRKMRKMRKMRKMRKMR
KMCACGATATACTTGCCGTCCTCCTTC; SEQ ID NO:41)
zc60488 - 3′ reverse oligo for amplification of
MSP94 domain 1 or MPS94 domain 1 YADS and incor-
poration of NotI site compatible with phagemidX;
(ATAAGAATATGCATGCGGCCGCTTGTAGAAACAAGGGTGCAACATGA;
SEQ ID NO:42)
zc59857 - forward oligo for MSP94 domain 2 YADS
amplification and insertion into phagemid X;
(TATGCAGGATCCTCTACACCT; SEQ ID NO:43)
zc60489 - 3′ reverse oligo for amplification or
MSP94, MSP94 domain 2, or MSP94 domain 2 YADS
including a NotI site compatible with phagemid
X and GIII fusion.
(ATAAGAATATGCATGCGGCCGCTGATTATCCATTCACTGACAGA;
SEQ ID NO:44)

The following reactions were set up-utilizing Invitrogen Accuprime polymerase and buffers (cat# 12344-024). The primers were annealed to each other and extended to create a core double stranded fragment.

Fragment #1
Oligos: zc59201, zc59202
Thermocycle profile: 1 cycle-94° C. for 2 min.; 10 cycles -94° C. for 30 sec., 60° C. for 30 sec., 68° C. for 30 sec.; 1 cycle-72° C. for 7 min., 4° C. hold;

Fragment #2
Oligos: zc59859, zc59204
Thermocycle profile: 1 cycle-94° C. for 2 min.; 10 cycles -94° C. for 30 sec., 60° C. for 30 sec., 68° C. for 30 sec.; 1 cycle-72° C. for 7 min., 4° C. hold.

7 μl of the reactions above were utilized as template for a second round of reactions to extend the core sequence to include restriction enzyme site and affiliated sequences required for digest.

Fragment #1b
Oligos: zc59852 (see Example 3, supra), zc60488
Thermocycle profile: 1 cycle-94° C. for 2 min.; 12 cycles -94° C. for 30 sec., 60° C. for 30 sec., 68° C. for 30 sec; 1 cycle-72° C. for 7 min., 4° C. hold;

Fragment #2b
Oligos: zc59857, zc60489
Thermocycle profile: 1 cycle-94° C. for 2 min.; 12 cycles -94° C. for 30 sec., 60° C. for 30 sec., 68° C. 30 sec.; 1 cycle-72° C. for 7 min., 4° C. hold.

The 1b and 2b PCR products were run on 2% low melt agarose TAE gels and the specific bands excised. The fragments were gel extracted utilizing the Qiagen Gel Extraction kit and eluted in H₂O Overnight BamHI and NotI restriction enzyme digestions were set up on the purified 1b and 2b fragments using New England Biotech reagents and buffers at 37° C.

The digested 1b and 2b PCR products were run on 2% low melt agarose TAE gels and the specific bands excised. The fragments were gel extracted utilizing the Qiagen Gel Extraction kit and eluted in the kits EB buffer.

Fifty micrograms of a large purified prep of phagemidxst2 with an irrelevant sequence insert was digested overnight with BamHI and NotI using New England Biotech reagents and buffers at 37° C. The digested phagemid vector was run on a 1% agarose TAE gels and the specific band excised. The fragment was gel extracted utilizing the Qiagen Gel Extraction kit and eluted in the kits EB buffer.

Ten microliter volume test ligations utilizing 100 ng of cut vector and 50 ng of digested MSP94 domain 1 (D1) YADS or MSP94 domain 2 (D2) YADS second round PCR fragments were set up overnight at 15° C. 1 μl of each was electroporated into 50 μl of the DH12s (Invitrogen electromax) bacterial strain using 1 mm cuvettes (2500 volts, 25 F, 100 ohms) outgrown in SOC media (Invitrogen) and plated on 100 μg/ml Amp plates. Colonies were counted to calculate ligation efficiency and clonal diversity. (T4 ligase and buffers from Invitrogen.)

Based on calculations from the small scale ligations and electroporations, larger ligations were set up to target of 1×10⁹ clonal diversity for each of D1 YADS and D2 YADS libraries using prepped fragments and vector used previously (5 μg D1 YADS fragment with 10 μg vector in 1 ml volume or 3 μg D2 fragment with 6 μg vector in 600 μl volume).

Ligations were ethanol precipitated in the presence of glycogen, and resuspended each in 10 μl of H₂O. The 10 μl of each resuspended ligation was added to 480 μl of DH12s electro competent cells, mixed and electroporated in 60 μl volumes in 1 mm cuvettes using 2.5 kV, 25 μF and 200 ohms settings. After each electroporation, the bacterial in the cuvette was resuspended in 1 ml of SOC media and transferred to a tube for outgrowth. The cuvette was then rinsed with an additional 0.5 mls of SOC and the volume was transferred to the outgrowth tube as well. All electroporations for D1 YADS or D2 YADS (8×1.5 ml for each) were outgrown for 1 hr at 37° C., 250 rpm and pooled. A small sample from the pooled cultures was taken, tittered, and plated to calculate diversity (D1 YADS=7.2×10⁸, D2 YADS=2.6×10⁹ total colony forming units for each).

The cells were centrifuged for 10 minutes at 2000 rpm on a tabletop centrifuge and the supernatant removed. The pellets were then resuspended in 24 ml of Low Phosphate media with 100 μg/ml Amp and the cultures outgrown for 2 hours at 37° C., 250 rpm in baffle flasks.

After 2 hours, 3 ml of M13KO7 helper phage were added to each of the D1 or D2 YADS, 24 ml cultures and mixed, and the culture then transferred to 293 ml of 37° C. pre-heated Low Phosphate media with 100 μg/ml Amp in a 2 L baffled shake flask. This culture was allowed to proceed for 2 hours at 37° C., 250 rpm. After 2 hours, kanamycin was added to a final concentration of 70 μg/ml to each flask and the cultures were allowed to proceed overnight at 30° C., 250 rpm.

The next day, the culture was centrifuged for 20 minutes at 8 kG to pellet bacteria and the supernatant saved. The supernatant (approximately 320 ml culture) was transferred to a new tube and the phage precipitated by addition of 62 mls of 20% (W/V) PEG 8000, 2.5M NaCl. The solutions were mixed and allowed to incubate in ice for approximately 2 hrs. The precipitated phage were each pelleted by centrifugation at 8 kG for 20 minutes and resuspended in 8 ml of PBS and titered.

The phage titers were calculated as 1×10¹³ cfu/ml for MSP94 domain 1 YADS and 2×10¹² cfu/ml for MSP94 domain 2 YADS.

### Example 15

### Screening of MSP94D1 YADS and MSP94D2 YADS Phase Libraries for Phase Binding to VEGF-A

The new larger, more diverse MSP94 domain 1 YADS and MSP94 domain 2 YADS phage libraries were screened for phage binding to VEGF-A₁₂₁ (121 amino acid isoform of VEGF-A), as described below.

### A. Panning Larger, More Diverse MSP94 Domain 1 YADS and Domain 2 YADS Phase Libraries for VEGF-A₁₂₁ Binders

The day previous to the enrichment panning, two sets of Nunc Immuno tubes were incubated with antibody. Two mls of PBS with 25 μg/ml of VEFG-A₁₂₁ (R & D Systems) was added to each of two tubes. Additionally, two mls of PBS with 25 μg/ml of PDGF-D (SEQ ID NO:45) was added to each of two tubes. The tubes were capped and tumbled slowly overnight at 4° C. The day previous to the phage panning, 2.5 mls of low phosphate media without antibiotics was inoculated with the TG1 bacterial strain and allowed to proceed overnight at 37° C., 250 RPM.

The VEGF-A₁₂₁- and PDGF-D-coated tubes were emptied, washed 5× with PBS 0.1% Tween 20 (PBS-T) and filled with 3 mls of PBS 0.1% Tween 20 (PBS-T) with 2% non-fat dry milk (blocking buffer). The tubes were capped and tumbled slowly at room temperature for 1 to 2 hrs.

Into two separate, labeled Nunc Immuno tubes, the following were added: 500 μl of PBS 0.1% Tween 20 (PBS-T) with 4% non-fat dry milk, 500 μl of the MSP94 Domain 1 YADS or MSP94 Domain 2 YADS phage library, and 1 ml of PBS 0.1% Tween 20 (PBS-T) with 2% non-fat dry milk. The tubes were capped and tumbled slowly at room temperature for 1 hour.

After 1 hour of blocking, PDGF-D-coated tubes were emptied and labeled, and the blocked phage mixtures were added. The tubes were capped and tumbled slowly at room temperature for an additional 1 hour.

After 1 hour of incubation, the VEGF-A₁₂₁-coated tubes were emptied of blocking buffer and labeled, and the phage mixtures from the Mu IgG isotype control antibody coated tubes were transferred to them. The tubes were capped and tumbled slowly at room temperature for an additional 1 hour.

After 1 hour of incubation, the phage mixtures were discarded, the tubes washed 5× with PBS 0.1% Tween 20 (PBS-T) and then 2× with PBS. The target-bound phage were eluted by the addition of 1 ml of 100 mM Triethyleneamine to the washed tubes. The tubes were capped and tumbled slowly at room temperature for 10 minutes. The eluted phage were then transferred to labeled Eppendorf tubes with 0.5 ml of 1M TRIS pH 7.4 for neutralization. A 50 μl sample was removed from each sample and titered as described in Example 13 above.

The TG1 culture that was started the day previous was utilized to start a larger 25 ml culture in low phosphate media without antibiotics at 0.03 OD₆₀₀. The culture was allowed to proceed in a baffled flask at 37° C., 250 RPM to an OD of 0.5. This culture was then utilized for phage titer and rescue.

The rest of the output phage volume was each separately added to 5 mls of 0.5 OD₆₀₀ TG1 culture, mixed and incubated without shaking at 37° C. for 30 minutes. After 30 min., 250 μl of M13KO7 helper phage (Invitrogen, >1×10¹¹ pfu/ml) was added to each tube, mixed, and incubated without shaking at 37° C. for 30 minutes. The infected cultures were then each added to 30° C. pre-warmed 25 mls of low phosphate media with 100 μg/ml Ampicillin in a baffled flask and allowed to proceed overnight at 30° C., 250 RPM. The cultures were then centrifuged at 8 kG for 20 minutes to pellet bacteria. The supernatant (˜30 mls) was transferred to a new tube and the phage precipitated by addition of 6 mls of 20% (W/V) PEG 8000, 2.5M NaCl. The solutions were mixed and allowed to incubate in ice for approximately 2 hours. The precipitated phage were each pelleted by centrifugation at 8 kG for 20 minutes and resuspended in 1 ml of PBS and titered.

A second round of panning using the first round amplified, precipitated phage stocks was performed as above. The second round pan and phage precipitation was performed as with the first round of panning over coated tubes pre-incubated with 25 μg/ml PDGF-D, while the concentration of VEGF-A₁₂₁ pre-incubation was dropped to 12.5 μg/ml.

A third round of panning using the second round amplified, precipitated phage stocks was performed as above. The third round pan and phage precipitation was performed as with the first and second round of panning over coated tubes pre-incubated with 25 μg/ml PDGF-D, while the concentration of VEGF-A₁₂₁ pre-incubation was dropped to 6.25 μg/ml.

Colonies from the third round pan elution were analyzed for VEGF-A₁₂₁ binding by ELISA.

### B. ELISA on Third Round Elution of MSP94 Domain 1 YADS and Domain 2 YADS VEGF-A₁₂₁ Binders

Two days before performing the ELISA, single colonies were picked with a toothpick from 100 μg/ml Amp plates from the third round phage elution titer plates of the new larger, more diverse MSP94 domain 1 YADS and MSP94 domain 2 YADS phage libraries panned over VEGF-A₁₂₁ (see section A, supra). The colonies were used to inoculate 500 μl of Low Phosphate w/Amp 100 media in a 96 deep well format. The plate was rotated at 235 rpm overnight at 37° C. with a gas permeable cover.

In the morning on the day before performing the ELISA, 5 μl of each well was transferred to a pre-heated round bottom shallow well plate with 100 μl of 37° C. Low Phosphate w/Amp 100 media/well. The plate was covered with a gas permeable cover and shaken at 350 rpm at 37° C. until the cultures reach OD₆₀₀ 0.5 (the original overnight plates were brought to 15% final conc of glycerol and frozen −80° C.). When the plates reached an OD₆₀₀ of 0.5, 50 μl of 5×10⁹ pfu/ml M13KO7 helper phage in Low phosphate media w/Amp 100 was added to each well and the plate was incubated without shaking 37° C. for 30 minutes. Fifty μl of Low phosphate media w/Amp 100/Kanamycin 100 μg/ml was then added to each well and the plate was allowed to shake overnight, 16 to 18 hrs at 37° C., 250 rpm.

On the day before performing the ELISA, Nunc Maxisorb 96 well flat bottom plates were obtained. VEGF-A₁₂₁ (R&D Systems, cat. # 298-VS/CF) was diluted to 1 μg/ml in PBS. The PDGF-D was diluted to 1 μg/ml in PBS. For each protein solution, 100 μl volumes were added to rows of separate plates (one set of plates for Domain 1 and Domain 2 each). The plate was incubated, covered overnight at 4° C.

After overnight incubation, the VEGF-A₁₂₁- and PDGF-D-coated plates were emptied by flick inversion and blotted on paper towels. 250 μl per well of PBS 0.1% Tween 20 (PBS-T) w/2.0% non-fat dry milk (blocking buffer) was added and the plate incubated for 1.5 hr at room temperature to block. The plates with the overnight cultures were centrifuged for 10 minutes at 3500 rpm on a table top centrifuge to pellet culture. The blocked plates were washed 3× with PBS-T using an automated plate washer. 66 μl of each phage stock supernatant from the “amplified phage” plate above for MSP94 domain 1 YADS and MSP94 domain 2 YADS and 33 μl of PBS-T with 3% milk was added to corresponding well sets for both the VEGF-A₁₂₁ and the PDGF-D plates. All wells were incubated for 1.5 hr at room temperature. The plates were washed 5× with PBS-T in an automated plate washer.

One hundred μl of a 1:5000 dilution in blocking buffer of an Anti-M13 antibody (Amersham 27-9421-01) was added to the sample wells. The plate was incubated for 1.5 hrs at room temperature and then washed 6× with PBS-T. 100 μl of TMB substrate was added to each well and after approximately 5-10 min. 50 μl of TMB stop solution was added to each well to stop the reaction.

The plate was read on a plate reader at 450 nM wavelength. The ELISA results indicated that (1) approximately 70% of the MSP94 domain 1 YADS VEGF-A₁₂₁ plate samples were positive with very low background signal on the PDGF-D plate; and (2)<1% MSP94 domain 2 YADS VEGF-A₁₂₁ plate samples were positive with respective background signal on the PDGF-D plate.

### Example 16

### Screening of MSP94D1 YADS and MSP94D2 YADS Phase Libraries for Phase Binding to PDGFRα

Day 1. 2 mL of 30 μg/mL human IgG-Fc (SEQ ID NO:46) was added to each of two immunotubes (Nunc), and 2 mL of 30 mg/mL PDGFRα-Fc (SEQ ID NO:47) was added to each of two immunotubes (Nunc). The tubes were capped and tumbled slowly overnight at 37° C.

Day 2. The coated immunotubes tubes were emptied, washed 5× with PBST (PBS; 0.1% Tween 20) and filled with 2 mls of blocking buffer (PBST; 2% non-fat dry milk). The tubes were capped and tumbled slowly at RT for 1 hour.

500 μl of the MSP94D1YADS and MSP94D2 YADS were blocked, in separate tubes, by the addition of 500 ul of PBST containing 4% non-fat dry milk, and 1 ml of PBST containing 2% non-fat dry milk. The tubes were capped and tumbled slowly for 1 hour at room temperature.

After 1 hour of blocking, the human IgG-Fc coated tubes were emptied, labeled and the blocked phage mixtures were added. The tubes were capped and tumbled slowly for 1 hour at room temperature.

The PDGFRα-coated tubes were emptied of blocking buffer. The phage mixtures from the human IgG-Fc coated tubes were then transferred to the PDGFRα-coated tubes, the tubes capped and tumbled slowly for 1 hour at room temperature.

After 1 hour of incubation, the phage mixtures were discarded, the tubes washed 1 5× with PBS-T and then 3× with PBS. The bound phage were eluted by the addition of 1 ml of 100 mM triethyleneamine to the washed tubes. The tubes were capped and tumbled slowly for 10 minutes at room temperature. The eluted phage were then transferred to culture tubes tubes containing 0.5 ml of 1M TRIS pH 7.4. A 50 ul sample was removed from each sample to determine the eluted phage titer.

The output phage were separately added to 5 mls of a 0.5 OD₆₀₀ TG1 culture, mixed and incubated for 30 minutes at 37° C. After 30 minutes, 250 μl of M13KO7 helper phage (Invitrogen) was added to each tube, mixed and incubated for 30 minutes at 37° C. The infected cultures were then transferred to baffled flasks containing 25 mL of 37° C. pre-warmed low phosphate media supplemented with ampicillin to 100 μg/ml. The cultures were incubated overnight on an orbital shaker, shaking at 250 RPM, at 30° C.

The next day, the culture was centrifuged for 20 min at 8000XG to pellet bacteria. The supernatant was transferred to a new tube containing 8 mls of 20% (W/V) PEG 8000, 2.5M NaCl. The solutions were mixed and allowed to incubate in ice for 2 hrs. The phage were pelleted by centrifugation at 8000XG for 20 minutes. The supernatant was discarded and the phage resuspended in 1 mL of PBS.

A 2nd round of panning using the first round amplified phage stocks was performed as above. For the second round pan, coated tubes pre-incubated with 30 μg/ml human IgG Fc, and then selected on tubes coated with 15 μg/mL PDGFRα-Fc.

A third round of panning using the second round amplified phage stocks was performed as above. For the third round pan, coated tubes pre-incubated with 30 μg/ml human IgG-Fc, and then selected on tubes coated with 7.5 μg/mL PDGFRα-Fc.

### Preparation of Phage for ELISA

Day 1. Single colonies were picked from the third round phage elution titer plates and transferred to a 96 deep well plate containing 500 μl of low phosphate media supplemented with ampicillin to 100 μg/mL. The plate was incubated overnight at 37° C. on a platform rotating at 225 RPM.

Day 2. 5 μl of each well was transferred to a pre-heated deep 96 well plate containing 200 μl of pre-heated 37° C. low phosphate media supplemented with ampicillin to 100 μg/mL. The plate was covered with a gas permeable cover and shaken at 350 rpm at 37° C. until the cultures reach OD₆₀₀ 0.5. When the cultures reach an OD₆₀₀ of 0.5, 50 μl of M13KO7 helper phage (Invitrogen) in low phosphate media supplemented with ampicillin to 100 μg/mL was added to each well and the plate was incubated at 37° C. for 30 minutes. 50 μl of low phosphate media supplemented with ampicillin to 100 μg/mL was then added to each well and the plate was incubated with shaking, over night at 30° C.

Day 3. Deep well plates were spun in a centrifuge at 5000XG for 20 minutes to pellet the cells. 25 ml of the cleared suernatants were analyzed by ELISA for their ability to specifically bind PDGFRα-Fc.

### PHAGE-ELISA for Identification of Specific Binders of PDGRα-Fc

Nunc Maxisorb 96 well flat bottom plates were incubated overnight at 4° C. with 100 μl/well of either 2 μg/mL human IgGFc or 2 μg/mL PDGFRα-Fc in 0.1M Carbonate pH 9.6. After overnight incubation, the plate was emptied by flick inversion and washed #X with PBST. 100 ml/well of blocking buffer was added to each well and the plate was incubated for one hour at room temperature to block. Following block 25 μl of phage stock from 96 well block was added to correlative wells on IgGFc and PDGFRα-Fc plates containing 75 μl PBST with 2.67% non-fat dry milk. Plates were incubated for 1 hour at room temperature and washed 3× with PBS-T.

Anti-M13 antibody (Amersham 27-9421-01) was diluted 1:5000 into PBST with 2% non-fat dry milk and 100 μl was added to each well. Plates were incubated for 1 hour at room temperature and washed 6× with PBS-T.

Plates were developed with TMB (BioFx Laboratories, Owings Mill, Md., cat. # TMBW-1000-01) as per manufacturer's directions and read on a plate reader at 450 nM. Elevated A₄₅₀ was revealed in 9 wells on the pdgfra plate with phage isolated from the MSP94D2 YADS library.

### Example 17

### Screening of the Large MSP94D1 YADS and MSP94D2 YADS Phage Libraries for Phage Binding to EGFR

Day 1. 2 mL of 30 μg/mL human IgG-Fc (SEQ ID NO:46) was added to each of two immunotubes (unc), and 2 mL of 30 mg/mL EGFR-Fc (R&D Systems) was added to each of two immunotubes (unc). The tubes were capped and tumbled slowly overnight at 37° C.

Day 2. The coated immunotubes tubes were emptied, washed 5× with PBST (PBS; 0.1% Tween 20) and filled with 2 mls of blocking buffer (PBST; 2% non-fat dry milk). The tubes were capped and tumbled slowly at RT for 1 hour.

500 μl of the MSP94D1YADS and MSP94D2 YADS were blocked, in separate tubes, by the addition of 500 ul of PBST containing 4% non-fat dry milk, and 1 ml of PBST containing 2% non-fat dry milk. The tubes were capped and tumbled slowly for 1 hour at room temperature.

After 1 hour of blocking, the human IgG-Fc coated tubes were emptied and labeled, and the blocked phage mixtures were added. The tubes were capped and tumbled slowly for 1 hour at room temperature.

The EGFR-coated tubes were emptied of blocking buffer. The phage mixtures from the human IgG-Fc-coated tubes were then transferred to the EGFR-Fc-coated tubes, the tubes capped and tumbled slowly for 1 hour at room temperature.

After 1 hour of incubation, the phage mixtures were discarded, the tubes washed 15× with PBS-T and then 3× with PBS. The bound phage were eluted by the addition of 1 ml of 100 mM triethyleneamine to the washed tubes. The tubes were capped and tumbled slowly for 10 minutes at room temperature. The eluted phage were then transferred to culture tubes tubes containing 0.5 ml of 1M TRIS pH 7.4. A 50 μl sample was removed from each sample to determine the eluted phage titer.

The output phage were separately added to 5 mls of a 0.5 OD₆₀₀ TG1 culture, mixed and incubated for 30 minutes at 37° C. After 30 minutes, 250 ul of M13KO7 helper phage (Invitrogen) was added to each tube, mixed and incubated for 30 minutes at 37° C. The infected cultures were then transferred to baffled flasks containing 25 mL of 37° C. pre-warmed low phosphate media supplemented with ampicillin to 100 μg/ml. The cultures were incubated overnight on an orbital shaker, shaking at 250 RPM, at 30° C.

The next day, the culture was centrifuged for 20 min at 8000XG to pellet bacteria. The supernatant was transferred to a new tube containing 8 mls of 20% (W/V) PEG 8000, 2.5M NaCl. The solutions were mixed and allowed to incubate in ice for 2 hrs. The phage were pelleted by centrifugation at 8000XG for 20 minutes. The supernatant was discarded and the phage resuspended in 1 mL of PBS.

A second round of panning using the first round amplified phage stocks was performed as above. For the second round pan coated tubes pre-incubated with 30 μg/ml human IgG-Fc, and then selected on tubes coated with 15 μg/mL EGFR-Fc.

A third round of panning using the second round amplified phage stocks was performed as above. For the third round, pan coated tubes pre-incubated with 30 μg/ml human IgG-Fc, and then selected on tubes coated with 7.5 μg/mL EGFR-Fc.

### Preparation of Phage for ELISA

Day 1. Single colonies were picked from the 3rd round phage elution titer plates and transferred to a 96 deep well plate containing 500 μl of low phosphate media supplemented with ampicillin to 100 μg/mL. The plate was incubated overnight at 37° C. on a platform rotating at 225 RPM.

Day 2. 5 ul of each well was transferred to a pre-heated deep 96 well plate containing 200 μl of pre-heated 37° C. low phosphate media supplemented with ampicillin to 100 μg/mL. The plate was covered with a gas permeable cover and shaken at 350 rpm at 37° C. until the cultures reach OD₆₀₀ 0.5. When the cultures reach an OD₆₀₀ of 0.5, 50 μl of M13KO7 helper phage (Invitrogen) in low phosphate media supplemented with ampicillin to 100 μg/mL was added to each well and the plate was incubated at 37° C. for 30 minutes. 50 μl of low phosphate media supplemented with ampicillin to 100 μg/mL was then added to each well and the plate was incubated with shaking, over night at 30° C.

Day 3. Deep well plates were spun in a centrifuge at 5000XG for 20 minutes to pellet the cells. 25 ml of the cleared suernatants were analyzed by ELISA for their ability to specifically bind EGFR-Fc.

### PHAGE-ELISA for Identification of Specific Binders of EGFR-Fc

Nunc Maxisorb 96 well flat bottom plates were incubated overnight at 4° C. with 100 μl/well of either 2 μg/mL human IgG-Fc (SEQ ID NO:52) or 2 μg/mL EGFR-Fc (R&D Systems) in 0.1M Carbonate pH 9.6. After overnight incubation, the plate was emptied by flick inversion and washed #X with PBST. 100 μl/well of blocking buffer was added to each well and the plate was incubated for one hour at room temperature to block. Following block 25 μl of phage stock from 96 well block was added to correlative wells on IgG-Fc and EGFR-Fc plates containing 75 μl PBST with 2.67% non-fat dry milk. Plates were incubated for 1 hour at room temperature and washed 3× with PBS-T.

Anti-M13 antibody (Amersham 27-9421-01) was diluted 1:5000 into PBST with 2% non-fat dry milk and 100 μl was added to each well. Plates were incubated for 1 hour at room temperature and washed 6× with PBS-T.

Plates were developed with TMB (BioFx Laboratories, Owings Mill, Md., cat. # TMBW-1000-01) as per manufacturer's directions and read on a plate reader at 450 nM. Elevated A₄₅₀ was revealed in 77 wells on the EGFR-Fc plate with phage isolated from the MSP94D1 YADS library, and 1 well on EGFR-Fc plate with the MSP94D2 YADS library.

From the foregoing, it will be appreciated that, although specific embodiments of the invention have been described herein for purposes of illustration, various modifications may be made without deviating from the spirit and scope of the invention. Accordingly, the invention is not limited except as by the appended claims. All publications, patents, and patent applications cited herein are hereby incorporated by reference in their entireties for all purposes.

What is claimed is:

1. A polypeptide comprising at least one of an MSP monomer domain I variant and an MSP monomer domain II variant, wherein
(I) said MSP monomer domain I variant is characterized in that
(a) in its unmodified form, the MSP monomer domain I comprises the amino acid sequence:
(x₁)C₂{x₃₋₇}{Z_{I}}C₁₈{x₁₉₋₃₆}C₃₇x₃₈x₃₉C₄₀x₄₁C₄₂{Z_{II}}x₄₈C₄₉C₅₀(x₅₁₋₅₅) (SEQ ID NO:59), said sequence representing a polypeptide region from a wild-type MSP protein corresponding to amino acid residues 1 to 55 of human MSP (SEQ ID NO:4), wherein {Z_{I}} and {Z_{II}} denote loop regions corresponding, respectively, to amino acids 8-17 and 43-47 of human MSP; and
(b) in its modified form, the MSP monomer domain I variant
(i) comprises cysteine residues C₂, C₁₈, C₄₀, C₄₂, C₄₉, and C₅₀;
(ii) comprises at least one of a modified {Z_{I}} and a modified {Z_{II}} loop region, wherein each modified loop region is a heterologous polypeptide segment of from 4 to 30 amino acids; and
(iii) comprises an amino acid sequence at least 60% identical to the wild-type MSP polypeptide region, exclusive of {Z_{I}} and {Z_{II}}; and
(II) said MSP monomer domain II variant is characterized in that
(a) in its unmodified form, the MSP monomer domain II comprises the amino acid sequence:
{x_{54-56}{Z}_{III}}C₆₄{x₆₅₋₇₂}C₇₃{x₇₄₋₇₇}{Z_{IV}}x₈₆C₈₇({x₈₈₋₉₄}) (SEQ ID NO:60), said sequence representing a polypeptide region from a wild-type MSP protein corresponding to amino acid residues 52 to 94 of human MSP (SEQ ID NO:4), wherein {Z_{III}} and {Z_{IV}} denote loop regions corresponding, respectively, to amino acids 57-63 and 78-85 of human MSP; and
(b) in its modified form, the MSP monomer domain II variant
(i) comprises cysteine residues C₆₄ and C₈₇;
(ii) comprises at least one of a modified {Z_{III}} and a modified {Z_{IV}} loop region, wherein each modified loop region is a heterologous polypeptide segment of from 4 to 30 amino acids; and
(iii) comprises an amino acid sequence at least 60% identical to the wild-type MSP polypeptide region, exclusive of {Z_{II}} and {Z_{IV}}.

2. The polypeptide of claim 1, which comprises one MSP monomer domain variant.

3. The polypeptide of claim 1, which is a multimer comprising a plurality of MSP monomer domain variants, each MSP monomer domain variant independently selected from the domain I variant and the domain II variant.

4. The polypeptide of claim 1, wherein
if the polypeptide comprises the MSP domain I variant, a non-sulfhydryl-containing amino acid residue is substituted for C₃₇; and
if the polypeptide comprises the MSP domain II variant, a non-sulfhydryl-containing amino acid residue is substituted for C₇₃.

5. The polypeptide of claim 1, which comprises both the MSP domain I variant and the MSP domain II variant.

6. The polypeptide of claim 5, wherein the domain I and domain II variants are linked via a polypeptide linker heterologous to the wild-type MSP protein.

7. The polypeptide of claim 5, wherein the domain II variant is linked C-terminal and directly adjacent to the domain I variant.

8. The polypeptide of claim 7, wherein the polypeptide comprises the following amino acid sequence:
(SEQ ID NO:72)
(S)CYFIPN{Z_{I}}CMDLKGNKHPINSEWQTDNxETCTC{Z_{II}}SCCT
LVSTP{Z_{III}}CQRIFKKEDxKYIV{Z_{IV}}TC(SVSEWII).

9. The polypeptide of claim 7, wherein the domain I variant comprises cysteine C₃₇ and the domain II variant comprises cysteine C₇₃.

10. The polypeptide of claim 9, wherein the polypeptide comprises the following amino acid sequence:
(SEQ ID NO:73)
(S)CYFIPN{Z_{I}}CMDLKGNKHPINSEWQTDNCETCTC{Z_{II}}SCCT
LVSTP{Z_{III}}CQRIFKKEDCKYIV{Z_{IV}}TC(SVSEWII).

11. The polypeptide of claim 1, wherein the wild-type MSP protein is the human MSP protein of SEQ ID NO:4.

12. The polypeptide of claim 1, which comprises the domain I variant.

13. The polypeptide of claim 12, comprising both the modified {Z_{I}} and the modified {Z_{II}} loop regions.

14. The polypeptide of claim 12, comprising only one of the modified {Z_{I}} and the modified {Z_{II}} loop regions.

15. The polypeptide of claim 5, wherein the wild-type MSP protein is the human MSP protein of SEQ ID NO:4.

16. The polypeptide of claim 5, wherein the domain I variant comprises the following amino acid sequence:
(SEQ ID NO:74)
(x)Cxxxxx{Z_{I}}CxDxxGxxHxxxxxWx(x)xxxxxCxC{Z_{II}}xCC
(xxxxx).

17. The polypeptide of claim 16, wherein the domain I variant comprises the following amino acid sequence:
(SEQ ID NO:75)
(x)C[yfs][fliqt][imqek][prnil][nlrp]{Z_{I}}CxDx[kd]
GxxHxx[ndg][ste]xWx([tn])xxxxxCxC{Z_{II}}[sitra]
CC(xxxxx).

18. The polypeptide of claim 17, wherein the domain I variant comprises the following amino acid sequence:
(SEQ ID NO:76)
(x)C[ys][fli][imqe][prn][nlr]{Z_{I}}CxDx[kd]GxxHx[il]
[nd][st]xW[qkr](T)[dek]xxxxCxC{Z_{II}}[sit]CC(xxxxx).

19. The polypeptide of claim 16, wherein the domain I variant comprises the following amino acid sequence:
(SEQ ID NO:77)
(S)CxxxPN{Z_{I}}CxDLKGNKHPxxSxWxTxxxxxCxC{Z_{II}}
xCC([ts]Lxx[ti]).

20. The polypeptide of claim 19, wherein the domain I variant comprise the following amino acid sequence:
(SEQ ID NO:78)
(S)C[ys][fl][im]PN{Z_{I}}C[mt]DLKGNKHP[il][nd]S[ekr]
W[qkr]T[de][nd]x[de]xCxC{Z_{II}}[si]CC([ts]L[vi][sa]
[ti]).

21. The polypeptide of claim 20, wherein the domain I variant comprise the following amino acid sequence:
(SEQ ID NO:79)
(S)CYFIPN{Z_{I}}CMDLKGNKHPINSEWQTDNxETCTC{Z_{II}}
SCC(TLVST).

22. The polypeptide of claim 1, which comprises the domain II variant.

23. The polypeptide of claim 22, comprising both the modified {Z_{III}} and the modified {Z_{IV}} loop regions.

24. The polypeptide of claim 22, comprising only one of the modified {Z_{III}} and the modified {Z_{IV}} loop regions.

25. The polypeptide of claim 5, wherein the wild-type MSP protein is the human MSP protein of SEQ ID NO:4.

26. The polypeptide of claim 5, wherein the domain II variant comprises the following amino acid sequence:
(SEQ ID NO:81)
xxx{Z_{III}}CxxxFxxxxxxxxV{Z_{IV}}xC(xxxxxxx).

27. The polypeptide of claim 26, wherein the domain II variant comprises the following amino acid sequence:
(SEQ ID NO:82)
xxx{Z_{III}}C[qrd][rkv][iq]F[knh]x[ek]xx[krt][yi]
[ist]V{Z_{IV}}xC(x[vi]xxWxx).

28. The polypeptide of claim 26, wherein the domain II variant comprises the following amino acid sequence:
(SEQ ID NO:83)
xxx{Z_{III}}CxxIFxxExxxxxV{Z_{IV}}xC(xxxxxxx).

29. The polypeptide of claim 28, wherein the domain II variant comprises the following amino acid sequence:
(SEQ ID NO:84)
[sa][ti]P{Z_{III}}C[qr][rk]xF[kn][kq]x[det]x[kr][yi]
[is]V{Z_{IV}}[te]C₈₇(x[vi]xxxxx).

30. The polypeptide of claim 26, wherein the domain II variant comprises the following amino acid sequence:
(SEQ ID NO:85)
xTP{Z_{III}}CQRIFKKExxKYIV{Z_{IV}}TC(xxxxWIx).

31. The polypeptide of claim 30, wherein the domain II variant comprises the following amino acid sequence:
(SEQ ID NO: 86)
[sa]TP{Z_{III}}CQRIFKKE[de]xKYIV{Z_{IV}}TC(xxx[eq]
WI[il]).

32. The polypeptide of claim 31, wherein the domain II variant comprises the following amino acid sequence:
(SEQ ID NO:87)
STP{Z_{III}}C₆₄QR1FKKEDx₇₃KYIV{Z_{IV}}TC₈₇(SVSEWII).

33. A polynucleotide encoding the polypeptide of claim 1.

34. A vector comprising the polynucleotide of claim 33.

35. The vector of claim 34, which is an expression vector.

36. The vector of claim 35, which is a phage display vector.

37. A host cell comprising the vector of claim 34.

38. A polypeptide library comprising:
a pool of different polypeptides, wherein each polypeptide of the polypeptide pool is a polypeptide as in claim 1; and wherein at least one of {Z_{I}}{Z_{II}}{Z_{III}}, and {Z_{IV}} is different among different polypeptides of the polypeptide pool.

39. The polypeptide library of claim 38, wherein each polypeptide of the polypeptide pool comprises one MSP monomer domain variant.

40. The polypeptide library of claim 38, wherein each polypeptide of the polypeptide pool comprises a domain I variant.

41. The polypeptide library of claim 38, wherein each polypeptide of the polypeptide pool comprises a domain II variant.

42. The polypeptide library of claim 38, wherein each polypeptide comprises at least two MSP monomer domain variants.

43. The polypeptide library of claim 42, wherein each of the at least two MSP monomer domain variants is a domain I variant.

44. The polypeptide library of claim 42, wherein each of the at least two MSP94 monomer domains is a domain II variant.

45. The polypeptide library of claim 42, wherein each polypeptide comprises both a domain I variant and a domain II variant.

46. The polypeptide library of claim 45, wherein the domain I and domain II variants of each polypeptide are linked via a polypeptide linker heterologous to the wild-type MSP protein.

47. The polypeptide library of claim 45, wherein each polypeptide is a polypeptide as in claim 7 or 8.

48. A method of screening a MSP monomer domain variant for the ability to bind to a target molecule, the method comprising:
(1) contacting a first target molecule with a MSP monomer domain variant (“first MSP monomer domain variant”), and
(2) determining whether the first MSP monomer domain variant specifically binds to the first target molecule;
wherein the first MSP monomer domain variant is selected from a domain I variant and a domain II variant, wherein
(I) the MSP monomer domain I variant is characterized in that
(a) in its unmodified form, the MSP monomer domain I comprises the amino acid sequence:
(x₁)C₂{x₃₋₇}{Z_{I}}C₁₈{x₁₉₋₃₆}C₃₇x₃₈x₃₉C₄₀x₄₁C₄₂{Z_{II}}x₄₈C₄₉C₅₀(x₅₁₋₅₅),
said sequence representing a polypeptide region from a wild-type MSP protein corresponding to amino acid residues 1 to 51 of human MSP (SEQ ID NO:4), wherein {Z_{I}} and {Z_{II}} denote loop regions corresponding, respectively, to amino acids 4-17 and 43-48 of human MSP; and
(b) in its modified form, the MSP monomer domain I variant
(i) comprises cysteine residues C₂, C₁₈, C₄₀, C₄₂, C₄₉, and C₅₀;
(ii) comprises at least one of a modified {Z_{I}} and a modified {Z_{II}} loop region, wherein each modified loop region is a heterologous polypeptide segment of from 4 to 30 amino acids; and
(iii) comprises an amino acid sequence at least 60% identical to the wild-type MSP polypeptide region, exclusive of {Z_{I}} and {Z_{II}}; and
(II) the MSP monomer domain II variant is characterized in that
(a) in its unmodified form, the MSP monomer domain II comprises the amino acid sequence:
{x₅₄₋₅₆}{Z_{III}}C₆₄{x₆₅₋₇₂}C₇₃{x₇₄₋₇₇}{Z_{IV}} x₈₆C₈₇({x₈₈₋₉₄}),
said sequence representing a polypeptide region from a wild-type MSP protein corresponding to amino acid residues 52 to 94 of human MSP (SEQ ID NO:4), wherein {Z_{II}} and {Z_{IV}} denote loop regions corresponding, respectively, to amino acids 56-63 and 79-86 of human MSP; and
(b) in its modified form, the MSP monomer domain II variant
(i) comprises cysteine residues C₆₄ and C₈₇;
(ii) comprises at least one of a modified {Z_{III}} and a modified {Z_{IV}} loop region, wherein each modified loop region is a heterologous polypeptide segment of from 4 to 30 amino acids; and
(iii) comprises an amino acid sequence at least 60% identical to the wild-type MSP polypeptide region, exclusive of {Z_{III}} and {Z_{IV}}.

49. The method of claim 48, wherein the first MSP monomer domain variant is the domain I variant.

50. The method of claim 49, wherein the domain I variant comprises both the modified {Z_{I}} and the modified {Z_{II}} loop regions.

51. The method of claim 49, wherein the domain I variant comprises only one of the modified {Z_{I}} and the modified {Z_{II}} loop regions.

52. The method of claim 48, wherein the first MSP monomer domain variant is the domain II variant.

53. The method of claim 52, wherein the domain II variant comprises both the modified {Z_{III}} and the modified {Z_{IV}} loop regions.

54. The method of claim 52, wherein the domain II variant comprises only one of the modified {Z_{III}} and the modified {Z_{IV}} loop regions.

55. The method of claim 48, wherein the first MSP monomer domain variant is from a polypeptide library as in claim 21 and the method comprises screening said polypeptide library for binding to the first target molecule.

56. The method of claim 48, wherein the first MSP monomer domain variant is identified as capable of specifically binding to the target molecule.

57. The method of claim 56, further comprising linking the first MSP monomer domain variant to a second monomer domain to form a multimer comprising at least two monomer domains.

58. The method of claim 57, wherein the second monomer domain is a second MSP monomer domain variant selected from a domain I variant and a domain II variant.

59. The method of claim 58, wherein the multimer comprises both a domain I variant and a domain II variant.

60. The method of claim 59, wherein the domain II variant is linked C-terminal and directly adjacent to the domain I variant.

61. The method of claim 60, wherein the domain I variant comprises cysteine C₃₇ and the domain II variant comprises cysteine C₇₃.

62. The method of claim 57, wherein the second monomer domain is a monomer domain identified as capable of binding to a second target molecule.

63. The method of claim 62, further comprising
contacting the multimer comprising the first and second monomer domains with the first and second target molecules; and
determining whether the multimer binds to the first and second target molecules.

64. The method of claim 57, wherein the second monomer domain is a monomer domain identified as capable of binding to the first target molecule.

65. The method of claim 64, further comprising
contacting the multimer comprising the first and second monomer domains with the first target molecule; and
determining whether the multimer binds to the first target molecule.

66. The method of claim 65, further comprising determining whether the multimer has an increased binding affinity or avidity for the first target molecule relative to the first MSP monomer domain variant.

67. The method of claim 57, which comprises linking the first MSP monomer domain variant to a library of second monomer domains to form a library of multimers, said library of multimers comprising a pool of multimers having different second monomer domains.

68. The method of claim 67, further comprising
contacting the library of multimers with the first target molecule; and
identifying a multimer that binds to the first target molecule.

69. The method of claim 68, further comprising determining whether the identified multimer binds to the first target molecule with greater affinity or avidity relative to the first MSP monomer domain variant.

70. The method of claim 55, wherein the first MSP monomer domain variant is identified as capable of specifically binding to the target molecule, and wherein the method further comprises
contacting the polypeptide library with a second target molecule;
identifying a second MSP monomer domain variant that binds to the second target molecule; and
linking the first and second MSP monomer domain variants to form a multimer comprising both the first and second MSP monomer domain variants.

71. The method of claim 70, further comprising
contacting the multimer with the first and second target molecules; and
determining whether the multimer binds to both the first and second target molecules.

72. A polypeptide comprising at least one of an MSP monomer domain I variant and an MSP monomer domain II variant, wherein
(I) said MSP monomer domain I variant is characterized in that
(a) in its unmodified form, the MSP monomer domain I comprises the amino acid sequence:
(x₁)C₂{x₃₋₇}{Z_{I}}C₁₈x₁₉x₂₀ {Z_{V}}{x₂₆₋₃₆}C₃₇x₃₈x₃₉C₄₀x₄₁C₄₂{Z_{II}}x₄₈C₄₉C₅₀(x₅₁₋₅₅) (SEQ ID NO:59), said sequence representing a polypeptide region from a wild-type MSP protein corresponding to amino acid residues 1 to 55 of human MSP (SEQ ID NO:4), wherein {Z_{I}}, {Z_{II}}, and {Z_{V}} denote loop regions corresponding, respectively, to amino acids 8-17, 43-47, and 21-25 of human MSP; and
(b) in its modified form, the MSP monomer domain I variant
(i) comprises cysteine residues C₂, C₁₈, C₄₀, C₄₂, C₄₉, and C₅₀;
(ii) comprises at least one of a modified {Z_{I}}, modified {Z_{II}}, and modified {Z_{V}} loop region, wherein each modified loop region is a heterologous polypeptide segment of from 4 to 30 amino acids; and
(iii) comprises an amino acid sequence at least 60% identical to the wild-type MSP polypeptide region, exclusive of {Z_{I}}, {Z_{II}}, and {Z_{V}}; and
(II) said MSP monomer domain II variant is characterized in that
(a) in its unmodified form, the MSP monomer domain II comprises the amino acid sequence:
{x₅₄₋₅₆}{Z_{III}}C₆₄{x₆₅₋₇₂}C₇₃{x₇₄₋₇₇}{Z_{IV}}x₈₆C₈₇({x₈₈₋₉₄}) (SEQ ID NO:60), said sequence representing a polypeptide region from a wild-type MSP protein corresponding to amino acid residues 52 to 94 of human MSP (SEQ ID NO:4), wherein {Z_{III}} and {Z_{IV}} denote loop regions corresponding, respectively, to amino acids 57-63 and 78-85 of human MSP; and
(b) in its modified form, the MSP monomer domain II variant
(i) comprises cysteine residues C₆₄ and C₈₇;
(ii) comprises at least one of a modified {Z_{III}} and a modified {Z_{IV}} loop region, wherein each modified loop region is a heterologous polypeptide segment of from 4 to 30 amino acids; and
(iii) comprises an amino acid sequence at least 60% identical to the wild-type MSP polypeptide region, exclusive of {Z_{III}} and {Z_{IV}}.

73. The polypeptide of claim 72, which comprises one MSP monomer domain variant.

74. The polypeptide of claim 72, which is a multimer comprising a plurality of MSP monomer domain variants, each MSP monomer domain variant independently selected from the domain I variant and the domain II variant.

75. The polypeptide of claim 72, wherein
if the polypeptide comprises the MSP domain I variant, a non-sulfhydryl-containing amino acid residue is substituted for C₃₇; and
if the polypeptide comprises the MSP domain II variant, a non-sulfhydryl-containing amino acid residue is substituted for C₇₃.

76. The polypeptide of claim 72, which comprises both the MSP domain I variant and the MSP domain II variant.

77. The polypeptide of claim 76, wherein the domain I and domain II variants are linked via a polypeptide linker heterologous to the wild-type MSP protein.

78. The polypeptide of claim 76, wherein the domain II variant is linked C-terminal and directly adjacent to the domain I variant.

79. The polypeptide of claim 78, wherein the polypeptide comprises the following amino acid sequence:
(SEQ ID NO:89)
(S)CYFIPN{Z_{I}}CMD{Z_{V}}HPINSEWQTDNxETCTC{Z_{II}}
SCCTLVSTP{Z_{III}}CQRIFKKEDxKYIV{Z_{IV}}TC(SVSEWII).

80. The polypeptide of claim 78, wherein the domain I variant comprises cysteine C₃₇ and the domain II variant comprises cysteine C₇₃.

81. The polypeptide of claim 80, wherein the polypeptide comprises the following amino acid sequence:
(SEQ ID NO:90)
(S)CYFIPN{Z_{I}}CMD{Z_{V}}HPINSEWQTDNCETCTC{Z_{II}}
SCCTLVSTP{Z_{III}}CQRIFKKEDCKYIV{Z_{IV}}TC(SVSEWII).

82. The polypeptide of claim 72, wherein the wild-type MSP protein is the human MSP protein of SEQ ID NO:4.

83. The polypeptide of claim 72, which comprises the domain I variant.

84. The polypeptide of claim 76 or 83, wherein the wild-type MSP protein is the human MSP protein of SEQ ID NO:4.


SEQUENCE LISTING (92 sequences)

SEQ ID NO: 1 - DNA, 345 bp, Homo sapiens
atgaatgttctcctgggcagcgttgtgatctttgccaccttcgtgactttatgcaatgcatcatgctatttcatacctaatgagggagttccaggagattcaaccaggaaatgcatggatctcaaaggaaacaaacacccaataaactcggagtggcagactgacaactgtgagacatgcacttgctacgaaacagaaatttcatgttgcacccttgtttctacacctgtgggttatgacaaagacaactgccaaagaatcttcaagaaggaggactgcaagtatatcgtggtggagaagaaggacccaaaaaagacctgttctgtcagtgaatggataatctaa

SEQ ID NO: 2 - protein, 114 aa, Homo sapiens
MNVLLGSVVIFATFVTLCNASCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 3 - DNA, 282 bp, Homo sapiens
tcatgctatttcatacctaatgagggagttccaggagattcaaccaggaaatgcatggatctcaaaggaaacaaacacccaataaactcggagtggcagactgacaactgtgagacatgcacttgctacgaaacagaaatttcatgttgcacccttgtttctacacctgtgggttatgacaaagacaactgccaaagaatcttcaagaaggaggactgcaagtatatcgtggtggagaagaaggacccaaaaaagacctgttctgtcagtgaatggataatc

SEQ ID NO: 4 - protein, 94 aa, Homo sapiens
SCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 5 - DNA, 73 bp, Artificial Sequence
  oligonucleotide primer zc58846
gcatctatgttcgttttttctattgctacaaacgcgtatgcaagtgcacagtcatgctatttcatacctaatg

SEQ ID NO: 6 - DNA, 74 bp, Artificial Sequence
  Oligonucleotide primer zc58847
gatcagcttttgttcggatccagcggccgaagcatcagcccgagcggccgcgattatccattcactgacagaac

SEQ ID NO: 7 - DNA, 73 bp, Artificial Sequence; identical to SEQ ID NO: 5
  Oligonucleotide primer zc58846

SEQ ID NO: 8 - DNA, 95 bp, Artificial Sequence
  Oligonucleotide primer zc58862
tcatgctatttcatacctaatgagggagttccaggagattcaaccaggaaatgcatggatctcaaaggaaacaacacccaataaactcggagtgg

SEQ ID NO: 9 - DNA, 99 bp, Artificial Sequence
  Oligonucleotide primer zc58863
tgtagaaacaagggtgcaacatgaaatttctgtttcgtagcaagtgcatgtctcaccgttgtcagtctgccactccgagtttattgggtgtttgtttcc

SEQ ID NO: 10 - DNA, 71 bp, Artificial Sequence
  Oligonucleotide primer zc58865
gatcagcttttgttcggatccagcggccgaagcatcagcccgagcggccgctgtagaaacaagggtgcaac

SEQ ID NO: 11 - DNA, 73 bp, Artificial Sequence
  Oligonucleotide primer zc58864
gcatctatgttcgttttttctattgctacaaacgcgtatgcaagtgcacagacacctgtgggttatgacaaag

SEQ ID NO: 12 - DNA, 123 bp, Artificial Sequence
  Oligonucleotide primer zc58861
tctacacctgtgggttatgacaaagacaactgccaaagaatcttcaagaaggaggacggcaagtatatcgtggtggagaagaaggacccaaaaaagacctgttctgtcagtgaatggataatc

SEQ ID NO: 13 - DNA, 74 bp, Artificial Sequence; identical to SEQ ID NO: 6
  Oligonucleotide primer zc58847

SEQ ID NO: 14 - DNA, 5634 bp, Artificial Sequence
  pARRB013
ccgacaccatcgaatggtgcaaaacctttcgcggtatggcatgatagcgcccggaagagagtcaattcagggtggtgaataagctttggagattatcgtcactgcaatgcttcgcaatatggcgcaaaatgaccaacagcggttgattgatcaggtagagggggcgctgtacgaggtaaagcccgatgccagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtacatggagaaaataaaatgaagaaaaacatcgcttttcttcttgcatctatgttcgttttttctattgctacaaacgcgtatgcacccgggcgggctgatgcttcggccgctggatccgaacaaaagctgatctcagaagaagacctaaactcacatcaccatcaccatcactaatgttttggcggatgagataagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttgcggccgcaatggccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggaccgcggccgcaatggccgccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgataaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgcagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgaaatt

SEQ ID NO: 15 - DNA, 4398 bp, Artificial Sequence
  PhagemidXST2 nucleotide sequence-starting at EcoRI and running through the Mlu and stop codon after gene 3
gaattcgggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccgcaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaagctttggagattatcgtcactgcaatgcttcgcaatatggcgcaaaatgaccaacagcggttgattgatcaggtagagggggcgctgtacgaggtaaagcccgatgccagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtacatggagaaaataaaatgaagaaaaacatcgcttttcttcttgcatctatgttcgttttttctattgctacaaatgcgtatgcaggatccggtaccgcggccgccatcaccatcatcaccatacgcgtgggagtggcggtgccgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaaacgcgttga

SEQ ID NO: 16 - DNA, 33 bp, Artificial Sequence
  Oligonucleotide primer zc59852
atgcatggatcctcatgctatttcatacctaat

SEQ ID NO: 17 - DNA, 39 bp, Artificial Sequence
  Oligonucleotide primer zc59853
atgcatgcggccgcttgtagaaacaagggtgcaacatga

SEQ ID NO: 18 - DNA, 36 bp, Artificial Sequence
  Oligonucleotide primer zc59883
atgcatggatcctctacacctgtgggttatgacaaa

SEQ ID NO: 19 - DNA, 36 bp, Artificial Sequence
  Oligonucleotide primer zc59854
atgcatgcggccgctgattatccattcactgacaga

SEQ ID NO: 20 - DNA, 393 bp, Artificial Sequence
  MSP94-CHis nucleotide coding sequence
atgaagaaaaacatcgcttttcttcttgcatctatgttcgttttttctattgctacaaatgcgtatgcaggatcctcatgctatttcatacctaatgagggagttccaggagattcaaccaggaaatgcatggatctcaaaggaaacaaacacccaataaactcggagtggcagactgacaactgtgagacatgcacttgctacgaaacagaaatttcatgttgcacccttgtttctacacctgtgggttatgacaaagacaactgccaaagaatcttcaagaaggaggactgcaagtatatcgtggtggagaagaaggacccaaaaaagacctgttctgtcagtgaatggataatcagcggccgccatcaccatcatcaccatacgcgttga

SEQ ID NO: 21 - protein, 130 aa, Artificial Sequence
  MSP94-Chis
MKKNIAFLLASMFVFSIATNAYAGSSCYFIPNEGVPGDSTRKCMDLKGNKHPINSEWQTDNCETCTCYETEISCCTLVSTPVGYDKDNCQRIFKKEDCKYIVVEKKDPKKTCSVSEWIISGRHHHHHHTR

SEQ ID NO: 22 - DNA, 276 bp, Artificial Sequence
  MSP94D1-CHis nucleotide coding sequence
atgaagaaaaacatcgcttttcttcttgcatctatgttcgttttttctattgctacaaatgcgtatgcaggatcctcatgctatttcatacctaatgagggagttccaggagattcaaccaggaaatgcatggatctcaaaggaaacaaacacccaataaactcggagtggcagactgacaacggtgagacatgcacttgctacgaaacagaaatttcatgttgcacccttgtttctacaagcggccgccatcaccatcatcaccatacgcgttga

SEQ ID NO: 23 - protein, 91 aa, Artificial Sequence
  MSP94D1-CHis
MKKNIAFLLASMFVFSIATNAYAGSSCYFIPNEGVPGDSTRKCMDLKGNKHPINSEWQTDNGETCTCYETEISCCTLVSTSGRHHHHHHTR

SEQ ID NO: 24 - DNA, 234 bp, Artificial Sequence
  MSP94D2-CHis nucleotide coding sequence
atgaagaaaaacatcgcttttcttcttgcatctatgttcgttttttctattgctacaaatgcgtatgcaggatcctctacacctgtgggttatgacaaagacaactgccaaagaatcttcaagaaggaggacggcaagtatatcgtggtggagaagaaggacccaaaaaagacctgttctgtcagtgaatggataatcagcggccgccatcaccatcatcaccatacgcgttga

SEQ ID NO: 25 - protein, 77 aa, Artificial Sequence [sequence omitted]

SEQ ID NO: 26 - DNA, 4385 bp, Artificial Sequence
  PhagemidXDSBA nucleotide sequence-starting at EcoRI and running through the Mlu and stop codon after gene 3
gaattcgggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccgcaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaagctttggagattatcgtcactgcaatgcttcgcaatatggcgcaaaatgaccaacagcggttgattgatcaggtagagggggcgctgtacgaggtaaagcccgatgccagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttatttttaatgtatttgtacatggagaaaataaaatgaaaaagatttggctggcgctggctggtttagttttagcgtttagcgcatcggcgggatccggtaccgcggccgccatcaccatcatcaccatacgcgtgggagtggcggtgccgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaaacgcgttga

SEQ ID NO: 27 - DNA, 96 bp, Artificial Sequence
  Oligonucleotide primer zc59201
tcatgctatttcatacctaatkmykmykmykmykmykmykmykmykmykmytgcatggatctcaaaggaaacaaacacccaataaactcggagtgg

SEQ ID NO: 28 - DNA, 98 bp, Artificial Sequence
  Oligonucleotide primer zc59202
tgtagaaacaagggtgcaacatgarkmrkmrkmrkmrkmgcaagtgatgtctcaccgttgtcagtctgccactccgagtttattgggtgtttgtttcc

SEQ ID NO: 29 - DNA, 84 bp, Artificial Sequence
  Oligonucleotide primer zc59859
tatgcaggatcctctacacctkmykmykmykmykmykmykmytgccaaagaatcttcaagaaggaggacggcaagtatatcgtg

SEQ ID NO: 30 - DNA, 76 bp, Artificial Sequence
  Oligonucleotide primer zc59204
gattatccattcactgacagaacaggtrkmrkmrkmrkmrkmrkmrkmrkmcacgatatacttgccgtcctccttc

SEQ ID NO: 31 - DNA, 47 bp, Artificial Sequence
  Oligonucleotide primer zc60488
ataagaatatgcatgcggccgcttgtagaaacaagggtgcaacatga

SEQ ID NO: 32 - DNA, 21 bp, Artificial Sequence
  Oligonucleotide primer zc59857
tatgcaggatcctctacacct

SEQ ID NO: 33 - DNA, 44 bp, Artificial Sequence
  Oligonucleotide primer zc60489
ataagaatatgcatgcggccgctgattatccattcactgacaga

SEQ ID NO: 34 - DNA, 165 bp, Artificial Sequence
  MSP94D1YADS nucleotide coding sequence (for mature MSP94D1YADS)
tcatgctatttcatacctaatkmykmykmykmykmykmykmykmykmykmytgcatggatctcaaaggaaacaaacacccaataaactcggagtggcagactgacaacggtgagacatgcacttgckmykmykmykmykmytcatgttgcacccttgtttctaca

SEQ ID NO: 35 - protein, 55 aa, Artificial Sequence
  MSP94D1YADS (mature protein)
SCYFIPNXXXXXXXXXXCMDLKGNKHPINSEWQTDNGETCTCXXXXXSCCTLVST

SEQ ID NO: 36 - DNA, 123 bp, Artificial Sequence
  MSP94D2YADS nucleotide coding sequence (for mature MSP94D1YADS)
tctacacctkmykmykmykmykmykmykmytgccaaagaatcttcaagaaggaggacggcaagtatatcgtgkmykmykmykmykmykmykmykmyacctgttctgtcagtgaatggataatc

SEQ ID NO: 37 - protein, 41 aa, Artificial Sequence
  MSP94D2YADS (mature protein)
STPXXXXXXXCQRIFKKEDGKYIVXXXXXXXXTCSVSEWII

SEQ ID NO: 38 - DNA, 96 bp, Artificial Sequence; identical to SEQ ID NO: 27
  Oligonucleotide primer zc59201

SEQ ID NO: 39 - DNA, 99 bp, Artificial Sequence
  Oligonucleotide primer zc59202
tgtagaaacaagggtgcaacatgarkmrkmrkmrkmrkmgcaagtgcatgtctcaccgttgtcagtctgccactccgagtttattgggtgtttgtttcc

SEQ ID NO: 40 - DNA, 84 bp, Artificial Sequence; identical to SEQ ID NO: 29
  Oligonucleotide primer zc59859

SEQ ID NO: 41 - DNA, 76 bp, Artificial Sequence; identical to SEQ ID NO: 30
  Oligonucleotide primer zc59204

SEQ ID NO: 42 - DNA, 47 bp, Artificial Sequence; identical to SEQ ID NO: 31
  Oligonucleotide primer zc60488

SEQ ID NO: 43 - DNA, 21 bp, Artificial Sequence
  Oligonucleotide primer zc59857
tatgcaggatcctctacacct

SEQ ID NO: 44 - DNA, 44 bp, Artificial Sequence; identical to SEQ ID NO: 33
  Oligonucleotide primer zc60489

SEQ ID NO: 45 - protein, 339 aa, Homo sapiens
RDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPRYLEYMPMD

SEQ ID NO: 46 - protein, 232 aa, Homo sapiens
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 756 aa, Artificial Sequence
  PDGFRa-Fc (mature protein)
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITILEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSEEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 240 aa, Artificial Sequence
  MSP domain I variant
XCYFIPNXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCMDLKGNKHPINSEWQTDNXETCTCXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXSCCXXXXX

SEQ ID NO: 49 - protein, 226 aa, Artificial Sequence
  MSP domain II variant
STPXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCQRIFKKEDXKYIVXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXTCXXXXXXX

SEQ ID NO: 50 - protein, 15 aa, Artificial Sequence
  Peptide linker
GGGGSGGGGSGGGGS

SEQ ID NO: 51 - protein, 17 aa, Artificial Sequence
  Peptide linker
GXXXXXGXXXXXGXXXX

SEQ ID NO: 52 - protein, 11 aa, Artificial Sequence
  Peptide linker
GGGXGGGXGGG

SEQ ID NO: 53 - protein, 25 aa, Artificial Sequence
  Peptide linker
GXXXXXXXXXXXCGXXXXXXXXXXX

SEQ ID NO: 54 - protein, 11 aa, Artificial Sequence
  Peptide linker
GGGGGCGGGGG

SEQ ID NO: 55 - protein, 25 aa, Artificial Sequence
  Peptide linker
PXXXXXXXXXXXCPXXXXXXXXXXX

SEQ ID NO: 56 - protein, 11 aa, Artificial Sequence
  Peptide linker
PPPPPCPPPPP

SEQ ID NO: 57 - protein, 19 aa, Artificial Sequence
  Peptide linker
GXXXXXXXNXXGXXXXXXX

SEQ ID NO: 58 - protein, 19 aa, Artificial Sequence
  Peptide linker
GXXXXXXXNXTGXXXXXXX

SEQ ID NO: 59 - protein, 55 aa, Artificial Sequence
  Generic reference sequence for unmodified form of MSP domain I
XCXXXXXXXXXXXXXXXCXXXXXXXXXXXXXXXXXXCXXCXCXXXXXXCCXXXXX

SEQ ID NO: 60 - protein, 41 aa, Artificial Sequence
  Generic reference sequence for unmodified form of MSP domain II
XXXXXXXXXXCXXXXXXXXCXXXXXXXXXXXXXCXXXXXXX

SEQ ID NO: 61 - protein, 240 aa, Artificial Sequence
  MSP domain I variant
XCXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXDXXGXXHXXXXXWXXXXXXXCXCXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCCXXXXX

SEQ ID NO: 62 - protein, 240 aa, Artificial Sequence; identical to SEQ ID NO: 61
  MSP domain I variant

SEQ ID NO: 63 - protein, 240 aa, Artificial Sequence; identical to SEQ ID NO: 61
  MSP domain I variant

SEQ ID NO: 64 - protein, 240 aa, Artificial Sequence
  MSP domain I variant
XCXXXPNXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXDLKGNKHPXXSXWXTXXXXXCXCXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCCXXXXX

SEQ ID NO: 65 - protein, 240 aa, Artificial Sequence
  MSP domain I variant
XCXXXPNXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXDLKGNKHPXXSXWXTXXXXXCXCXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCCXLXXX

SEQ ID NO: 66 - protein, 226 aa, Artificial Sequence
  MSP domain II variant
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXXXFXXXXXXXXVXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXXXXXXX

SEQ ID NO: 67 - protein, 226 aa, Artificial Sequence
  MSP domain II variant
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXXXFXXXXXXXXVXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXXXXWXX

SEQ ID NO: 68 - protein, 226 aa, Artificial Sequence
  MSP domain II variant
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXXIFXXEXXXXXVXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXXXXXXX

SEQ ID NO: 69 - protein, 226 aa, Artificial Sequence
  MSP domain II variant
XXPXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXXXFXXXXXXXXVXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXXXXXXX

SEQ ID NO: 70 - protein, 226 aa, Artificial Sequence
  MSP domain II variant
XTPXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCQRIFKKEXXKYIVXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXTCXXXXWIX

SEQ ID NO: 71 - protein, 226 aa, Artificial Sequence; identical to SEQ ID NO: 70
  MSP domain II variant

SEQ ID NO: 72 - protein, 184 aa, Artificial Sequence
  Full-length MSP variant (domain I and domain II)
XCYFIPNXXXXXXXXXXXXXXXXXXXXXXXXXXXXXXCMDLKGNKHPINSEWQTDNXETCTCXXXXXXXXXXXXXXXXXXXXXXXXXXXXXXSCCTLVSTPXXXXXXXXXXXXXXXXXXXXXXXXXXXXXXCQRIFKKEDXKYIVXXXXXXXXXXXXXXXXXXXXXXXXXXXXXXTCXXXXXXX

SEQ ID NO: 73 - protein, 184 aa, Artificial Sequence
  Full-length MSP variant (domain I and domain II)
XCYFIPNXXXXXXXXXXXXXXXXXXXXXXXXXXXXXXCMDLKGNKHPINSEWQTDNCETCTCXXXXXXXXXXXXXXXXXXXXXXXXXXXXXXSCCTLVSTPXXXXXXXXXXXXXXXXXXXXXXXXXXXXXXCQRIFKKEDCKYIVXXXXXXXXXXXXXXXXXXXXXXXXXXXXXXTCXXXXXXX

SEQ ID NO: 74 - protein, 100 aa, Artificial Sequence
  MSP domain I variant
XCXXXXXXXXXXXXXXXXXXXXXXXXXXXXXXXXXXXCXDXXGXXHXXXXXWXXXXXXXCXCXXXXXXXXXXXXXXXXXXXXXXXXXXXXXXXCCXXXXX

SEQ ID NO: 75 - protein, 100 aa, Artificial Sequence; identical to SEQ ID NO: 74
  MSP domain I variant

SEQ ID NO: 76 - protein, 100 aa, Artificial Sequence; identical to SEQ ID NO: 74
  MSP domain I variant

SEQ ID NO: 77 - protein, 100 aa, Artificial Sequence
  MSP domain I variant
XCXXXPNXXXXXXXXXXXXXXXXXXXXXXXXXXXXXXCXDLKGNKHPXXSXWXTXXXXXCXCXXXXXXXXXXXXXXXXXXXXXXXXXXXXXXXCCXXXXX

SEQ ID NO: 78 - protein, 100 aa, Artificial Sequence; identical to SEQ ID NO: 77
  MSP domain I variant

SEQ ID NO: 79 - protein, 100 aa, Artificial Sequence
  MSP domain I variant
XCYFIPNXXXXXXXXXXXXXXXXXXXXXXXXXXXXXXCMDLKGNKHPINSEWQTDNXETCTCXXXXXXXXXXXXXXXXXXXXXXXXXXXXXXSCCXXXXX

SEQ ID NO: 80 - protein, 55 aa, Artificial Sequence
  MSP domain I variant
XCYFIPNXXXXXXXXXXCMDLKGNKHPINSEWQTDNXETCTCXXXXXSCCXXXXX

SEQ ID NO: 81 - protein, 86 aa, Artificial Sequence
  MSP domain II variant
XXXXXXXXXXXXXXXXXXXXXXXXXXXXXXXXXCXXXFXXXXXXXXVXXXXXXXXXXXXXXXXXXXXXXXXXXXXXXXCXXXXXXX

SEQ ID NO: 82 - protein, 86 aa, Artificial Sequence; identical to SEQ ID NO: 81
  MSP domain II variant

SEQ ID NO: 83 - protein, 86 aa, Artificial Sequence
  MSP domain II variant
XXXXXXXXXXXXXXXXXXXXXXXXXXXXXXXXXCXXIFXXEXXXXXVXXXXXXXXXXXXXXXXXXXXXXXXXXXXXXXCXXXXXXX

SEQ ID NO: 84 - protein, 86 aa, Artificial Sequence
  MSP domain II variant
XXPXXXXXXXXXXXXXXXXXXXXXXXXXXXXXXCXXXFXXXXXXXXVXXXXXXXXXXXXXXXXXXXXXXXXXXXXXXXCXXXXXXX

SEQ ID NO: 85 - protein, 86 aa, Artificial Sequence
  MSP domain II variant
XTPXXXXXXXXXXXXXXXXXXXXXXXXXXXXXXCQRIFKKEXXKYIVXXXXXXXXXXXXXXXXXXXXXXXXXXXXXXTCXXXXXXX

SEQ ID NO: 86 - protein, 86 aa, Artificial Sequence; identical to SEQ ID NO: 85
  MSP domain II variant

SEQ ID NO: 87 - protein, 86 aa, Artificial Sequence
  MSP domain II variant
STPXXXXXXXXXXXXXXXXXXXXXXXXXXXXXXCQRIFKKEDXKYIVXXXXXXXXXXXXXXXXXXXXXXXXXXXXXXTCXXXXXXX

SEQ ID NO: 88 - protein, 41 aa, Artificial Sequence
  MSP domain II variant
STPXXXXXXXCQRIFKKEDXKYIVXXXXXXXXTCXXXXXXX

SEQ ID NO: 89 - protein, 209 aa, Artificial Sequence
  Full-length MSP variant (domain I and domain II, with variable loop Zv)
XCYFIPNXXXXXXXXXXXXXXXXXXXXXXXXXXXXXXCMDXXXXXXXXXXXXXXXXXXXXXXXXXXXXXXHPINSEWQTDNXETCTCXXXXXXXXXXXXXXXXXXXXXXXXXXXXXXSCCTLVSTPXXXXXXXXXXXXXXXXXXXXXXXXXXXXXXCQRIFKKEDXKYIVXXXXXXXXXXXXXXXXXXXXXXXXXXXXXXTCXXXXXXX

SEQ ID NO: 90 - protein, 209 aa, Artificial Sequence
  Full-length MSP variant (domain I and domain II, with variable loop Zv)
XCYFIPNXXXXXXXXXXXXXXXXXXXXXXXXXXXXXXCMDXXXXXXXXXXXXXXXXXXXXXXXXXXXXXXHPINSEWQTDNCETCTCXXXXXXXXXXXXXXXXXXXXXXXXXXXXXXSCCTLVSTPXXXXXXXXXXXXXXXXXXXXXXXXXXXXXXCQRIFKKEDCKYIVXXXXXXXXXXXXXXXXXXXXXXXXXXXXXXTCXXXXXXX

SEQ ID NO: 91 - protein, 55 aa, Artificial Sequence
  MSP94 domain I variant
SCYFIPNXXXXXXXXXXCMDLKGNKHPINSEWQTDNXETCTCXXXXXSCCTLVST

SEQ ID NO: 92 - protein, 41 aa, Artificial Sequence
  MSP94 domain II variant
STPXXXXXXXCQRIFKKEDXKYIVXXXXXXXXTCSVSEWII